(19) United States
(12) Patent Application Publication
(10) Pub. No.: US 2015/0313913 A1
(43) Pub. Date: Nov. 5, 2015

(54) POSITIVE ALLOSTERIC MODULATORS OF THE GABA-A RECEPTOR IN THE TREATMENT OF AUTISM

(71) Applicant: UNIVERSITY OF WASHINGTON THROUGH ITS CENTER FOR COMMERCIALIZATION, Seattle, WA (US)

(72) Inventors: William A. CATTERALL, Seattle, WA (US); Sung HAN, Seattle, WA (US)

(21) Appl. No.: 14/651,123

(22) PCT Filed: Feb. 4, 2014
(86) PCT No.: PCT/US14/14683

§ 371 Date: Jun. 10, 2015

Related U.S. Application Data
(60) Provisional application No. 61/760,874, filed on Feb. 5, 2013.
(60) Provisional application No. 61/927,365, filed on Jan. 14, 2014.

(51) Int. Cl. A61K 31/5513; A61K 31/5025

(52) U.S. Cl. CPC A61K 31/5513; A61K 31/5025

(57) ABSTRACT

Provided herein are methods and formulations for treating an Autism Spectrum Disorder using low doses of an agent that enhances signaling through the GABA receptor.

### GOVERNMENT SUPPORT

This invention was made with U.S. government support under RO1 NS25704, awarded by the National Institutes of Health. The U.S. Government has certain rights in the invention.

### BACKGROUND

Autism is a neurodevelopmental disorder that first appears during infancy or childhood, and generally follows a steady course without remission. Symptoms gradually begin after the age of six months, become established by age two or three years, and tend to continue through adulthood. Autism is distinguished not by a single symptom, but by a characteristic triad of symptoms: impairments in social interaction; impairments in communication; and restricted interests and repetitive behavior. Other aspects, such as atypical eating, are also common in autistic children.

Diagnosis of autism and autism spectrum disorders is based on behavior, rather than through identification of a mechanistic cause. Autism Spectrum Disorder is defined in the DSM-V as exhibiting (i) deficits in social communication and interaction not caused by general developmental delays (must exhibit three criteria including deficits in social-emotional reciprocity, deficits in nonverbal communication, and deficits in creating and maintaining relationships appropriate to developmental level), (ii) demonstration of restricted and repetitive patterns of behavior, interest or activities (must exhibit two of the following four criteria: repetitive speech, repetitive motor movements or repetitive use of objects, adherence to routines, ritualized patterns of verbal or nonverbal behavior, or strong resistance to change, fixated interests that are abnormally intense of focus, and over or under reactivity to sensory input or abnormal interest in sensory aspects of environment), (iii) symptoms must be present in early childhood, and (iv) symptoms collectively limit and hinder everyday functioning.

### SUMMARY

The present disclosure is generally directed at methods of rescuing autism-like behavior in Dravet syndrome, autism spectrum disorders, and in idiopathic autism itself, including cognitive impairment associated with Dravet Syndrome, autism-spectrum disorders, and idiopathic autism. In certain embodiments the methods include the administration of low doses of benzodiazepines or non-benzodiazepine drugs that increase the response of the GABA-A receptor to GABA (“non-BDZ GABA-A enhancers”). In certain embodiments, these include positive allosteric modulators of GABA-A receptor activity. In further embodiments, these include α2 and/or α3 selective GABA-A receptor positive allosteric modulators, e.g., as described herein in the Subunit Selective Positive Allosteric Modulators section.

In one aspect, provided herein is a method for treating an Autism Spectrum Disorder (ASD) or an indicium thereof, the method comprising administering a low dose of an agent that increases GABAergic signaling to a subject having an ASD, thereby treating the ASD in the subject.

In one embodiment of this aspect and all other aspects described herein, the agent that increases GABAergic signaling is a positive allosteric modulator of the GABA-A receptor.

In another embodiment of this aspect and all other aspects described herein, the positive allosteric modulator of the GAB A-A receptor has efficacy at a GAB A-A receptor comprising an α2 and/or α3 subunit.

In another embodiment of this aspect and all other aspects described herein, the positive allosteric modulator of the GABA-A receptor is selective for a GABA-A receptor comprising an α2 and/or α3 subunit.

In another embodiment of this aspect and all other aspects described herein, the agent that increases GABAergic signaling is a benzodiazepine, or a non-benzodiazepine enhancer at the GABA-A receptor (“non-BDZ GABA-A enhancer”).

In another embodiment of this aspect and all other aspects described herein, the benzodiazepine is a full agonist or a partial agonist at the GABA-A receptor.

In another embodiment of this aspect and all other aspects described herein, the benzodiazepine is a short-acting or long-acting benzodiazepine.

In another embodiment of this aspect and all other aspects described herein, the benzodiazepine is clonazepam or clobazam.

In another embodiment of this aspect and all other aspects described herein, the non-BDZ GABA-A enhancer is L838,417.

In another embodiment of this aspect and all other aspects described herein, the non-BDZ GABA-A enhancer is a neurosteroid.

In another embodiment of this aspect and all other aspects described herein, the dose of the agent that increases GABAergic signaling is less than the dose of the same agent that causes sedation, anticonvulsive effects, or anxiolytic effects in a subject.

In another embodiment of this aspect and all other aspects described herein, the dose of the agent that increases GABAergic signaling is about 10% of the dose of the same agent that causes sedation, anticonvulsive effects or anxiolytic effects in a subject.

In another embodiment of this aspect and all other aspects described herein, the ASD comprises at least one symptom selected from the group consisting of: poor social interactions, repetitive behaviors, cognitive deficit and impaired language development.

Another aspect provided herein relates to a method for reducing or ameliorating at least one indicium of an Autism Spectrum Disorder (ASD), the method comprising: administering a sub-sedative, sub-anxiolytic or sub-anticonvulsive dose of an agent that increases the response of the GABA-A receptor to GABA, whereby at least one indicium of an ASD is reduced or ameliorated.

In one embodiment of this aspect and all other aspects described herein, the at least one indicium of an Autism Spectrum Disorder is selected from the group consisting of: repetitive behavior(s), impaired social interactions, cognitive deficit and impaired language development.

In another embodiment of this aspect and all other aspects described herein, the at least one indicium of an Autism Spectrum Disorder is repetitive behavior(s) and/or impaired social interactions.

In another embodiment of this aspect and all other aspects described herein, the agent is a benzodiazepine, a neurosteroid, a subunit-selective positive allosteric modulator of GABA-A, or a non-BDZ GABA-A enhancer.

Another aspect provided herein relates to a composition comprising a low-dose formulation of a benzodiazepine and a pharmaceutically acceptable carrier, wherein the dose of the benzodiazepine is less than the dose of the same benzodiazepine that causes sedation, anticonvulsive effects or anxiolytic effects in a subject.

In one embodiment of this aspect and all other aspects described herein, the dose of the benzodiazepine is less than 20% of the dose of the same benzodiazepine that causes sedation, anticonvulsive effects or anxiolytic effects in a subject.

In another embodiment of this aspect and all other aspects described herein, the dose of the benzodiazepine is less than 10% of the dose of the same benzodiazepine that causes sedation, anticonvulsive effects or anxiolytic effects in a subject.

In another embodiment of this aspect and all other aspects described herein, the composition is in the form of a tablet, a capsule, a suspension, or a solution.

In another embodiment of this aspect and all other aspects described herein, the benzodiazepine is a full agonist or a partial agonist at the GABA-A receptor.

In another embodiment of this aspect and all other aspects described herein, the benzodiazepine has efficacy at a GABA-A receptor comprising an α2 and/or α3 subunit.

In another embodiment of this aspect and all other aspects described herein, the benzodiazepine is selective for a GABA-A receptor comprising an α2 and/or α3 subunit.

In another embodiment of this aspect and all other aspects described herein, the benzodiazepine is a short-acting or long-acting benzodiazepine.

In another embodiment of this aspect and all other aspects described herein, the benzodiazepine is clonazepam and the composition comprises a dose of clonazepam within the range of 0.01 mg to 0.05 mg.

Also provided herein, in another aspect, is a composition comprising a low-dose formulation of a non-benzodiazepine GABA-A enhancer (non-BDZ GABA-A enhancer) and a pharmaceutically acceptable carrier, wherein the dose of the non-BDZ GABA-A enhancer is less than the dose of the same non-BDZ GABA-A enhancer that causes sedation, anticonvulsive effects or anxiolytic effects in a subject.

In one embodiment of this aspect and all other aspects described herein, the dose of the non-BDZ GABA-A enhancer is less than 20% of the dose of the same non-BDZ GABA-A enhancer that causes sedation, anticonvulsive effects or anxiolytic effects in a subject.

In another embodiment of this aspect and all other aspects described herein, the dose of the non-BDZ GABA-A enhancer is less than 10% of the dose of the same non-BDZ GABA-A enhancer that causes sedation, anticonvulsive effects or anxiolytic effects in a subject.

In another embodiment of this aspect and all other aspects described herein, the non-BDZ GABA-A enhancer has efficacy at a GABA-A receptor comprising an α2 and/or α3 subunit.

In another embodiment of this aspect and all other aspects described herein, the non-BDZ GABA-A enhancer is selective for a GABA-A receptor comprising an α2 and/or α3 subunit.

In another embodiment of this aspect and all other aspects described herein, the non-BDZ GABA-A enhancer is L838,417.

In another embodiment of this aspect and all other aspects described herein, the non-BDZ GABA-A enhancer is a neurosteroid.

In another embodiment of this aspect and all other aspects described herein, the low dose formulation composition is in the form of a tablet, a capsule, a suspension, or a solution.

In another aspect, provided herein is a use of a formulation as described herein for the treatment of an Autism Spectrum Disorder or at least one indicium thereof.

Another aspect described herein is the use of a formulation as described herein for the preparation of medicament for the treatment of an Autism Spectrum Disorder or at least one indicium thereof.

Also provided herein, in another aspect, is a use of a low-dose formulation of an agent that increases GABAergic signaling for the treatment of an Autism Spectrum Disorder or at least on indicium thereof.

In one embodiment of this aspect and all other aspects described herein, the agent that increases GABAergic signaling is a positive allosteric modulator of the GABA-A receptor.

In another embodiment of this aspect and all other aspects described herein, the positive allosteric modulator of the GAB A-A receptor has efficacy at a GAB A-A receptor comprising an α2 and/or α3 subunit.

In another embodiment of this aspect and all other aspects described herein, the positive allosteric modulator of the GABA-A receptor is selective for a GABA-A receptor comprising an α2 and/or α3 subunits.

In another embodiment of this aspect and all other aspects described herein, the agent that increases GABAergic signaling is a benzodiazepine, or a non-benzodiazepine enhancer at the GABA-A receptor (non-BDZ GABA-A enhancer).

In another embodiment of this aspect and all other aspects described herein, the benzodiazepine is a full agonist or a partial agonist at the GABA-A receptor.

In another embodiment of this aspect and all other aspects described herein, the benzodiazepine is a short-acting or long-acting benzodiazepine.

In another embodiment of this aspect and all other aspects described herein, the benzodiazepine is clonazepam or clobazam. In another embodiment of this aspect and all other aspects described herein, the non-BDZ GABA-A enhancer is L838,417.

In another embodiment of this aspect and all other aspects described herein, the non-BDZ GABA-A enhancer is a neurosteroid.

In another embodiment of this aspect and all other aspects described herein, the dose of the agent that increases GABAergic signaling is less than the dose of the same agent that causes sedation, anticonvulsive effects, or anxiolytic effects in a subject.

In another embodiment of this aspect and all other aspects described herein, the dose of the agent that increases GABAergic signaling is about 10% of the dose of the same agent that causes sedation, anticonvulsive effects or anxiolytic effects in a subject.

In another embodiment of this aspect and all other aspects described herein, the ASD comprises at least one symptom selected from the group consisting of: poor social interactions, repetitive behaviors, cognitive deficit and impaired language development.

Another aspect provided herein relates to a use of a low-dose formulation of an agent that increases the response of the GABA-A receptor to GABA for reducing or ameliorating at least one indicium of an Autism Spectrum Disorder (ASD), the use comprising: administering a sub-sedative, sub-anxiolytic or sub-anticonvulsive dose of an agent that increases the response of the GABA-A receptor to GABA, whereby at least one indicium of an ASD is reduced or ameliorated.

### BRIEF DESCRIPTION OF THE DRAWINGS

FIGS. 1A-1L |Scn1a+/− mice show hyperactivity, anxiety-like behavior, increased stereotypes, and impaired social behavior. FIGS. 1A-1B: In the open field test, Scn1a+/− mice travel longer distances compared with wild-type mice (FIG. 1A) and spend less time in the centre (FIG. 1B). FIGS. 1C-1D: In the open field, Scn1a+/− mice spend more time grooming (FIG. 1C) and circling (FIG. 1D) than wild-type mice. In FIG. 1D, one complete turn is counted as one circle, regardless of direction. FIGS. 1E-1F: In the elevated plus maze, Scn1a+/− mice enter less frequently in the open arms (FIG. 1E) and spend less time in the open arms (FIG. 1F). FIGS. 1G-1H: Three-chamber experiment. FIG. 1G, Whereas wild-type mice spend more time in the chamber housing a stranger mouse (M) than the chamber housing an empty cage (E), Scn1a+/− mice have no preference for either chamber. FIG. 1H, Whereas wild-type mice spend more time in the chamber housing a novel mouse (M2) than in a chamber housing a familiar mouse (M1), Scn1a+/− mice have no preference for either chamber. FIGS. 1I-1L: Social interaction test. FIG. 1I, Scn1a+/− mice show decreased interaction with a caged stranger mouse when compared with wild-type mice. FIG. 1J, In a 10-min reciprocal interaction test, pairs of wild-type and Scn1a+/− unfamiliar mice had significantly less non-aggressive (Non-A) and aggressive (A) interactions than pairs of wild-type and wild-type stranger mice. Aggressive behaviors included attacking, wrestling and biting the dorsal surface, and non-aggressive behaviors include nose-to-nose sniffing, anogenital sniffing and grooming. FIG. 1K, Scn1a+/− mice move significantly less when they encountered the stranger mouse compared to an empty cage, whereas there is no difference in movement for wild type. FIG. 1L, Scn1a+/− mice, but not wild-type mice, show increased immobilization behavior in the presence of the caged stranger mouse than in the presence of an empty cage. All data shown are means±s.e.m. from 10-12 mice per genotype. *P<0.05, **P<0.01, ***P<0.001.

FIGS. 2A-2I |Profound deficits in context-dependent spatial learning and memory in Scn1a+/− mice. a, In the novel object recognition test, Scn1a+/− mice had normal recognition memory for a preconditioned object (F: Familiar), which was presented 24 h before the test, so that they spent more time with the novel object (N: Novel). b, Discrimination index, the normalized ratio of time spent with the familiar object divided by time spent with the novel object, shows that there is no difference between wild-type and Scn1a+/− mice for novel object recognition ability. c, In the contextual fear-conditioning test, Scn1a+/− mice had a normal fear response immediately after the training (Train), but showed a profound deficit in short-term (30 min) and long-term (24 h) memory for the spatial context associated with a 2-s mild foot shock (0.5 mA) when compared to wild-type mice. d, e, In the Barnes circular maze, Scn1a+/− mice had a profound deficit in spatial learning. Wild-type mice made fewer errors finding the target hole (d), and showed decreased latency to escape the maze (e) during the 4-day repeated training trials, but Scn1a+/− mice show no improvement in performance for either the number of errors made to find the target hole (d), or the time to escape the maze (e). f-i, During the probe trial on the 5th day, Scn1a+/− mice had a profound deficit in spatial memory. They spent significantly more time to find the target hole (g), poked the correct target hole with significantly lower frequency (h), and stayed significantly less time in the target area (i) when compared with wild-type mice, although total distance moved was not significantly different from that of wild-type mice (f). All data shown are means±s.e.m. from 6-10 mice per genotype. *P<0.05, ***P<0.001.

FIGS. 3A-3H |D1×1/2-Scn1a+/− mice have the impaired spatial learning and autism-related phenotypes observed in Scn1a+/− mice. FIG. 3A, In the open field test, Dlx1/2-Scn1a+/− mice moved farther compared to Cre-negative Scn1a/loxp littermates. FIG. 3B, In the open field test, Dlx1/2-Scn1a+/− mice spent less time in the centre. FIG. 3C, Dlx1/2-Scn1a+/− mice show increased circling behavior. One complete turn, regardless of direction was counted as one circling. FIGS. 3D, 3E, In the elevated plus maze, Dlx1/2-Scn1a+/− mice entered less frequently into open arms (FIG. 3D), and spent significantly less time in open arms (FIG. 3E). FIG. 3F, In the open field social interaction test, Dlx1/2-Scn1a+/− mice showed decreased interaction with social cues compared to Scn1a1/loxp littermates. FIG. 3G, In the 3-chamber test, Dlx1/2-Scn1a+/− mice had no preference for the stranger mouse. FIG. 3H, In the contextual fear conditioning test, Dlx1/2-Scn1a+/− mice had a normal fear response immediately after the foot shock during training but showed a profound deficit in short-term (30 min) and long-term (24 h) memory for the spatial context associated with a 2-s mild foot shock (0.5 mA) when compared to wild-type mice. Dlx, Dlx1/2-Scn1a+/− mice. Flox, Cre-negative Scn1a/loxp mice. E, Empty cage. C, Center. M, Mouse. All data shown are means±s.e.m. from 7-9 mice per genotype. *P<0.05; **P<0.01; ***P<0.001.

FIGS. 4A-4F |Deficit of NaV1.1 channels and GABAergic neurotransmission in Scn1a+/− hippocampal GABAergic interneurons. Immunocytochemical staining of forebrain neurons from 10-month old mice for NaV1.1 channels. FIG. 4A, Co-immunolabelling of NaV1.1 and GABA revealed co-expression of NaV1.1 and GABA in the hippocampal CA1 region in wild-type mice. FIG. 4B, Co-immunolabelling of NaV1.1 and GABA revealed decreased expression of NaV1.1 channels in GABAergic interneurons in the forebrain of Scn1a+/− mice. FIG. 4C, Example traces of sIPSC from wild-type and Scn1a+/− hippocampal CA1 neurons. FIG. 4D, Example traces of sEPSC from wild-type and Scn1a+/− hippocampal CA1 neurons. FIG. 4E, Cumulative plot and average values (inset) of sIPSC frequency. The frequency of sIPSC is decreased, but the amplitude of sIPSC is unchanged in Scn1a+/− hippocampal CA1 slices when compared to wild-type slices (FIG. 30A). FIG. 4F, Cumulative plot and average values (inset) of sEPSC frequency. The frequency of sEPSC is increased, but the amplitude of sEPSC is unchanged in Scn1a+/− hippocampal CA1 slices when compared to wild-type slices (FIG. 30B). mPFC, medial prefrontal cortex. MC, motor cortex. SC, sensory cortex. PC, parietal cortex. CA1, hippocampal CA1 region. All data shown are mean±s.e.m. from 15-19 recordings per genotype. **P<0.01.

FIGS. 5A-5G |Complete rescue of impaired social behavior and fear-associated memory deficits in Scn1a+/− mice by low-dose clonazepam (CLZ) treatment. FIG. 5A, Both wild-type and Scn1a+/− mice showed dose-dependent sedation by CLZ. Maximal concentration of CLZ without sedative or anxiolytic effect was 0.0625 mg/kg. FIGS. 5B, 5C, In the open field social interaction test (FIG. 5B) and 3-chamber social preference test (FIG. 5C), decreased social interaction in Scn1a+/− mice was completely restored by a single intraperitoneal injection of 0.0625 mg/kg CLZ 30 min before the test. This CLZ effect on social interaction completely disappeared after 1 week of clearance in the same Scn1a+/− mice. CLZ effects on social interaction were absent in wild-type mice. Pre, pre CLZ treated; Post, post CLZ-treated. FIGS. 5D, 5E, In the contextual fear-conditioning test, a single intraperitoneal injection of 0.0625 mg/kg CLZ, 30 min before the training, led to a complete rescue of short-term (30 min) and long-term (24 h) fear-associated contextual memory in Scn1a+/− mice (FIG. 5E), but no significant change of fear-associated contextual memory by CLZ was observed in wild-type mice (FIG. 5D). All data shown are means±s.e.m. from 6-12 mice per genotype. n.s., not significant. FIG. 5F, Cumulative plot and average value (inset) of sIPSC amplitude. Treatment with 10 μM CLZ increased the amplitude of sIPSC, but the frequency of sIPSC was unchanged by 10 μM CLZ in Scn1a+/− hippocampal CA1 slices (FIG. 37A). FIG. 5G, Cumulative plot and average value (inset) of sEPSC frequency. Treatment with 10 μM CLZ decreased the frequency of sEPSC, but the amplitude of sEPSC was unchanged by 10 μM CLZ in Scn1a+/− hippocampal CA1 slices (FIG. 37B). All data shown are means±s.e.m. from 15-20 recordings per treatment group. *P<0.05, **P<0.01, ***P<0.001.

FIGS. 6A-6D Increase in social interaction of BTBR mice by low-dose CLZ. In the three-chamber test, a plexiglass box with three chambers connected by passageways is used. The center chamber is empty, the left chamber contains an empty mouse cage, and the right chamber contains an identical mouse cage with a stranger mouse inside. The test mouse is placed in the center chamber and the movement of the test mouse is monitored with a digital camera and video tracking software (Noldus Ethovision). Test mice were injected with the indicated doses of CLZ i.p. 30 min before the experiment, and their movement was monitored for 10 min. FIG. 6A. Time spent in each chamber by the test mouse. FIG. 6B. Ratio of time spent in each chamber. FIG. 6C. Interaction Time with stranger mouse. FIG. 6D. Ratio of time spent with mouse vs. object.

FIGS. 7A-7D. Reversibility of the effect of CLZ on social interaction in BTBR mice. The social interaction test was performed as in FIGS. 6A-6D. Mice were tested with no prior treatment on Day 0 (Pre). After seven days (Day 7), mice were injected with 0.050 mg/kg CLZ 30 min, and the three-chamber test was performed as before. Finally on Day 14, the three-chamber test was performed without drug treatment (Post). FIG. 7A. BTBR mice. Time spent in each chamber. FIG. 7B. BTBR mice. Time of interaction with stranger mouse. FIG. 7C. Control C57Bl/6 mice. Time spent in chamber. FIG. 7D. Time of interaction with stranger mouse.

FIGS. 8A-8C Social interaction and stereotyped behavior in an open field in BTBR mice. Control (C57Bl/6) and BTBR mice were allowed to interact with a stranger mouse in an open field. A digital camera and quantitative video tracking analysis was used to measure interaction and stereotyped circling. Tests were done on day 0 with no drug treatment (Pre), Day 7 with CLZ treatment, and day 14 (Post). FIG. 8A. Nose-to-nose interaction. FIG. 8B. Total interaction time. FIG. 8C. Total number of circles.

FIGS. 9A-9B. Effects of clobazam on social interaction behavior of BTBR mice. BTBR mice were treated with vehicle (CON), CLZ, or clobazam (CBZ) by i.p. injection 30 min before a three-chamber test for social interaction. Movements of the test mice were recorded with a digital camera and analyzed quantitatively with video-tracking software. FIG. 9A. Time in each chamber. FIG. 9B. Interaction time with object or stranger mouse.

FIG. 10 Effect of CLZ on context-dependent fear conditioning in BTBR mice. Mice freeze into a motionless position in response to fear, providing a measure of their fear behavior using movement-tracking methods. Mice were allowed to habituate for 30 min to a new cage in which the floor has an electrical grid for delivering a mild shock and there are unique markings for easy recognition. After the habituation period, the motion of the mice is recorded for two min and the percent of time freezing is measured (Control). A mild foot shock (0.5 mAmp) is delivered, and freezing behavior is recorded for another two min (Train). The mice are returned to the home cage. After 30 min in home cage, the mice are returned to the shock cage, and their freezing behavior is measured (30 min). The mice are again returned to home cage. After 24 h, the mice are returned to the shock cage and freezing behavior is recorded (24 h).

FIG. 11. The chemical structure of L-838,417.

FIG. 12. Effect of L838,417 on social behavior of BTBR mice. The three-chamber test for social interaction was performed as described.⁶¹ The indicated doses of L838,417 were administered by i.p. injection 30 min before the three-chamber test. Time spent in center chamber with no objects, in the left chamber with an inanimate object (an empty cage), and in the right chamber with a stranger mouse in an identical small cage were measured with video tracking methods and analyzed using Noldus software. Note the convex versus concave biphasic dose-response relationship for time in the chamber with the mouse, with peak at 0.05 mg/kg L838,417, versus time in the chamber with the inanimate object, with low point at 0.05 mg/kg.

FIGS. 13A-13B. Effect of L838,417 on context-dependent fear conditioning in BTBR mice. FIG. 13A Left. WT mice were injected with saline or 0.05 mg/kg of L838,417 i.p. as indicated. After 30 min, mice were allowed to habituate in a shock cage (Control), and freezing behavior was measured by video recording and Noldus data analysis. They were then subjected to a mild foot shock (Train), and fear-related freezing behavior was measured.⁶¹ Mice were returned to home cages. After 30 min or 24 h, the mice were returned to the shock cage and their freezing behavior was measured again. FIG. 13B Right. DS mice were subjected to the same experimental protocol. Note that DS mice fail to remember the spatial context of the fearful event (the foot shock) (CON, blue), whereas a single dose of L838,417 administered 30 min before Training and again before the 24-h test period substantially rescued spatial learning behavior.

FIG. 14 Representative tracks for open field test. WT and Scn1a+/− mice were allowed to explore novel open field arena (38 cm×42 cm) for 10 min. Scn1a+/− mice explore much more at the boundary of the arena, and much less in the center (15 cm×15 cm square imaginary area) compared with WT mice.

FIG. 15 Representative tracks for elevated plus maze. WT and Scn1a+/− mice were allowed to explore an elevated plus maze for 10 min. Scn1a+/− mice spend more time in the closed arm (C), and less time in the open arm (0) compared with WT mice.

FIGS. 16A-16B Histogram of repetitive behaviors. FIG. 16A, Grooming times for WT and Scn1a+/− mice (FIG. 1E) are replotted as a histogram. FIG. 16B, Circling behavior data of WT and Scn1a+/− mice (FIG. 1F) are replotted as a histogram. In both cases, all individual Scn1a+/− mice have longer grooming times and more circling than the mean for WT mice (WT: 28 s for grooming; 22 circles).

FIG. 17 Nest building test. Scn1a+/− mice have a decreased nest-building index compared with WT mice. All values are means±s.e.m. from 9-10 mice per genotype. *P<0.05, **P<0.01, ***P<0.001.

FIG. 18 Representative tracks for the 3-chamber test. Representative video-tracks of mice for each three-chamber experiment, which measures the amount of time that the mouse spends in the chamber with an empty cage (E: Empty), in the center (C: Center) or in the chamber with either a stranger or familiar mouse (M: Mouse). Top row, WT mice; bottom row, Scn1a+/− mice.

FIG. 19 Three-chamber test for social preference. Both WT and Scn1a+/− mice have no preference for either side of three-chamber unit in the habituation period. E, Empty. C, Center. All data shown are means±s.e.m. from 10-12 mice per genotype.

FIGS. 20A-20C Social preference in the three-chamber test. FIG. 20A, Both WT and Scn1a+/− mice had no preference for either side of three-chamber unit in the habituation period. FIG. 20B, In the three-chamber test, Scn1a+/− mice had no preference for the stranger mouse, whereas WT mice interacted a longer time with the stranger mouse than with the empty cage. FIG. 20C, Scn1a+/− mice had no preference for novel mouse, compared with familiar mouse, whereas WT mice spent a longer time with novel mouse. E, Empty. M, Mouse. All data shown are means±s.e.m. from 10-12 mice per genotype. **P<0.01, ***P<0.001.

FIGS. 21A-21B Representative tracks in the open field social interaction test. FIG. 21A, Representative video-tracks of mice in the open field social interaction test. Left column, WT mice; right column, Scn1a+/− mice. Top row, empty cage is placed (E); bottom row, mouse is placed in the cage (M). FIG. 21B, Tracks from a Scn1a+/− mouse that displayed immobilization behavior when a stranger mouse is encountered. This example shows an unusually long single period of immobilization (bottom).

FIGS. 22A-22B Social-interaction test in the open field. FIGS. 22A, 22B, When presented both an object and a stranger mouse simultaneously, only WT mice preferred to stay in the quadrant where the caged stranger mouse was placed (FIG. 22A), and preferred to interact more with the caged stranger mouse (FIG. 22B). Scn1a+/− mice had no preference for the stranger mouse; instead, they display a tendency toward avoidance of the stranger mouse (p=0.09). 0, Object. M, Mouse. All data shown are means±s.e.m. from 7 mice per genotype. *P<0.05, **P<0.01.

FIGS. 23A-23D Olfactory discrimination test using the three-chamber experimental paradigm. A three-chamber test was used to test the olfactory sensing in WT and Scn1a+/− mice. FIG. 23A, Both WT and Scn1a+/− mice spent more time in a chamber where the olfactory cue (food odor) was located. FIG. 23B, Both WT and Scn1a+/− mice preferred to interact with the food olfactory cue. FIG. 23C, Both WT and Scn1a+/− mice entered the olfactory cue chamber more frequently. FIG. 23D, Both WT and Scn1a+/− mice spent less time finding the olfactory cue chamber than the other chamber. NF, No Food odor. C, Center. F, Food odor. All data shown are means±s.e.m. from 9 mice per genotype. ***P<0.001.

FIGS. 24A-24F Olfactory preference test using social cues. FIGS. 24A, 24B, WT mice spent more time in the chamber where female bedding (FIG. 24A) or male bedding (FIG. 24B) was located. In contrast, Scn1a+/− mice displayed no difference in time spent between clean bedding and female bedding (FIG. 24A), and spent less time in the male bedding area compared with the clean bedding area (FIG. 24B). FIG. 24C, Close interaction of mice with social olfactory cues was calculated by subtracting the interaction time with a neutral cue from the interaction time with a social cue. Whereas WT mice spent more time with both male and female social cues, Scn1a+/− mice avoided interacting with the male social cue. FIGS. 24D, 24E, Whereas WT mice spent more time in the chamber with male bedding (FIG. 24D) or female bedding (FIG. 24E) than in the chamber with clean bedding, Scn1a+/− mice had no preference for either chamber. FIG. 24F, Both WT mice and Scn1a+/− mice displayed strong avoidance to fox urine, which had never been exposed before to the test mice. ‡, 95% Confidence Interval does not include zero. CB, Clean Bedding. C, Center. FB, Female Bedding. MB, Male Bedding. M, Male. F, Female. W, Water. FU, Fox Urine. All data shown are means±s.e.m. from 9 mice per genotype. *P<0.05, **P<0.01, ^{#,}***P<0.001.

FIGS. 25A-25D Olfactory habituation/dishabituation and olfactory choice tests. FIG. 25A, Whereas WT mice exhibited significant habituation and dishabituation to banana flavor (B), male mouse urine (U), and finely ground food pellet (F), Scn1a+/− mice exhibited no significant habituation and dishabituation to the odor stimuli, but exhibited strong habituation and dishabituation behavior to ground food pellet (F). FIG. 25B, Whereas WT mice displayed no digging behavior during odor presentation, Scn1a+/− mice displayed substantially increased digging behavior when banana flavor and male urine were presented indicating that these odors are aversive to Scn1a+/− mice. FIGS. 25C, 25D, Y-maze olfactory choice test. Whereas WT mice displayed strong preference to the odor-containing arm (FIG. 25C), Scn1a+/− mice displayed strong avoidance to the odor-containing arm (FIG. 25D). All data shown are means±s.e.m. from 7 mice per genotype. *P<0.05, **P<0.01, ***P<0.001, #P<0.05 for dishabituation; ‡P<0.05 for habituation.

FIGS. 26A-26C Behavioral parameters during contextual fear conditioning. Behavioral parameters, such as total distance moved (FIG. 26A), mean velocity (FIG. 26B), and maximum velocity (FIG. 26C) were measured during contextual fear conditioning (FIG. 3C). Scn1a+/− mice display substantially increased activities during both test sessions when compared with wild-type littermates. These increased activities are simply a reflection of the decreased freezing behaviors. However, the increased activity during test sessions is not greater than the activity during control session in Scn1a+/− mice, which indicates that Scn1a+/− mice display no panic-fleeing responses, and therefore do not perceive the shock chamber as a fearful context. All data shown are means±s.e.m from 9 mice per genotype. n.s., Not Significant (P>0.05).

FIGS. 27A-27E No behavioral phenotypes in the Dlx1/2-I12b-Cre transgenic mice. Open field (FIGS. 27A-27C), social preference tests (FIG. 27D), and contextual fear conditioning (FIG. 27E) were performed to test the effects of Cre transgene expression on behaviors. The data show that Dlx1/2-I12b-Cre transgenic mice display no autism-related phenotypes or impaired context-dependent fear memory, in contrast to those observed in the Dlx-Cre+Scn1a+/loxp mice. All data shown are means±s.e.m. from 7 mice per genotype.

FIG. 28 Co-expression of NaV1.1 and GABA in the prefrontal cortex. Co-immunolabeling of NaV1.1 and GABA revealed co-expression of NaV1.1 and GABA in the deep layer of prefrontal cortex in WT mice.

FIGS. 29A-29C Reduced expression of NaV1.1 channels in Scn1a+/− GABAergic interneurons. FIGS. 29A, 29B, Co-immunolabeling of NaV1.1 and GABA revealed reduced expression of NaV1.1 and GABA in the hippocampal CA1 region (FIG. 29A), and in the deep layer of prefrontal cortex (FIG. 29B) in Scn1a+/− mice. FIG. 29C, Average pixel density per cell was significantly decreased in the Scn1a+/−GABAergic neurons in the deep layer of prefrontal cortex. No reduction in the total number of GABAergic neurons per microscopic field was observed: hippocampal CA1 (n=19.6±0.4 for WT, n=20.2±0.4 for Scn1a+/−; p=0.41, Student's t-test); in prefrontal cortex (n=34.7±2.6 for WT, n=34.6±2.3 for Scn1a+/−; p=0.98, Student's t-test). All data shown are means±s.e.m. ***P<0.001.

FIGS. 30A-30D Intact synaptic functions in hippocampal neurons. FIG. 30A, example traces of miniature IPSCs recorded in CA1 pyramidal neurons from WT and Scn1a+/− mouse hippocampal slices. FIG. 30B, the amplitude and the frequency of miniature IPSCs were unchanged in Scn1a+/− hippocampal CA1 slices when compared to WT slices. FIG. 30C, example traces of miniature EPSCs recorded in CA1 pyramidal neurons from WT and Scn1a+/− mouse hippocampal CA1 region. FIG. 30D, the amplitude and the frequency of miniature EPSCs were unchanged in Scn1a+/− hippocampal CA1 slices when compared to WT slices. All data shown are means±s.e.m. from 9-11 recordings per genotype.

FIGS. 31A-31D Intact synaptic functions in prefrontal cortex neurons. FIG. 31A, example traces of miniature IPSCs recorded pyramidal neurons from WT and Scn1a+/− mouse prefrontal cortex slices. FIG. 31B, the amplitude and the frequency of miniature IPSCs were unchanged in Scn1a+/− prefrontal cortex slices when compared to WT slices. FIG. 31C, example traces of miniature EPSCs from prefrontal cortex of WT and Scn1a+/− mice. FIG. 31D, the amplitude and the frequency of miniature EPSCs were unchanged in Scn1a+/− prefrontal cortex slices when compared to WT slices. All data shown are means±s.e.m. from 11 recordings per genotype.

FIGS. 32A-32B Spontaneous neurotransmission onto hippocampal CA1 pyramidal neurons. FIG. 32A, Cumulative plot and average values (inset) of spontaneous IPSC amplitude. The amplitude of spontaneous IPSCs was unchanged in CA1 pyramidal cells in Scn1a+/− hippocampal slices when compared to WT slices. FIG. 32B, Cumulative plot and average values (inset) of spontaneous EPSC amplitude. The amplitude of spontaneous EPSCs was unchanged in Scn1a+/− hippocampal CA1 slices when compared to WT slices. All data shown are means±s.e.m. from 15-19 recordings per genotype.

FIGS. 33A-33D Spontaneous neurotransmission onto pyramidal cells in prefrontal cortex. FIG. 33A, example traces of spontaneous IPSCs from WT and Scn1a+/− prefrontal cortex slices. FIG. 33B, the frequency of spontaneous IPSCs was decreased, but the amplitude of spontaneous IPSCs was unchanged in Scn1a+/− prefrontal cortex slices when compared to WT slices. FIG. 33C, example traces of spontaneous EPSCs from WT and Scn1a+/− prefrontal cortex slices. FIG. 33D, the frequency of spontaneous EPSCs was increased, but the amplitude of spontaneous EPSCs was unchanged in pyramidal cells in Scn1a+/− prefrontal cortex slices when compared to WT slices. All data shown are means±s.e.m. from 14-16 recordings per genotype. *P<0.05, **P<0.01.

FIGS. 34A-34D No sedative or anxiolytic effect of low-dose CLZ Scn1a+/− mice. FIG. 34A, In the open field test, the locomotor activity of Scn1a+/− mice was not changed by 0.0625 mg/kg CLZ treatment. FIG. 34B, An anxiolytic effect was not observed in Scn1a+/− mice by 0.0625 mg/kg CLZ treatment. FIGS. 34C, 34D, An elevated plus maze test was performed to further test the anxiolytic effect of 0.0625 mg/kg CLZ on Scn1a+/− mice. Low-dose CLZ did not elicit anxiolytic effects on Scn1a+/− mice. Pre, Pre-CLZ treatment. CLZ, Clonazepam. Post, Post-CLZ treatment. CON, Control. All data shown are means±s.e.m. from 10-12 mice per genotype. There are no significant effects of CLZ treatment (P>0.05).

FIGS. 35A-35D Recovery of social interaction deficits by treatment with low-dose CLZ. FIGS. 35A, 35B, In the open-field social interaction test, the social interaction preference in WT mice was not changed by 0.0625 mg/kg CLZ injection, measured by time spent in the quadrant (FIG. 35A), or by close interaction time (FIG. 35B). FIGS. 35C, 35D, Scn1a+/− mice showed completely recovered social interaction behaviors after a 0.0625 mg/kg CLZ injection, measured by time spent in the quadrant (FIG. 35C), or by close interaction time (FIG. 35D), and the CLZ effect completely disappeared after a 1 week period of drug clearance in the same mice. E, Empty cage. M, Mouse. Pre, Pre-CLZ treatment. CLZ, Clonazepam. Post, Post-CLZ treatment. All data shown are means±s.e.m. from 10-11 mice per genotype. *P<0.05, **P<0.01, ***P<0.001.

FIGS. 36A-36D. Recovery of social preference deficits by treatment with low-dose CLZ. FIGS. 36A, 36B, In the three-chamber test, the social interaction preference in WT mice was not changed by 0.0625 mg/kg CLZ injection, measured by time spent in close interaction (FIG. 36A), or by time spent in the chamber (FIG. 36B). FIGS. 36C, 36D, Scn1a+/− mice showed completely recovered social interaction behaviors after a 0.0625 mg/kg CLZ injection, measured by time spent in the quadrant (FIG. 36C), or by time spent in the chamber (FIG. 36D), and the CLZ effect completely disappeared after a 1-week period of CLZ injection in the same mice. E, Empty cage. C, Center. M, Mouse. Pre, Pre-CLZ treatment. CLZ, Clonazepam. Post, Post-CLZ treatment. All data shown are means±s.e.m. from 10-12 mice per genotype. *P<0.05, **P<0.01, ***P<0.001.

FIGS. 37A-37B Effects of low-dose CLZ treatment on synaptic transmission recorded in CA1 pyramidal cells. FIG. 37A, Cumulative plot and average values (insets) of spontaneous IPSC frequency. The frequency of spontaneous IPSCs was unchanged by 10 μM CLZ in Scn1a+/− hippocampal CA1 slices. FIG. 37B, Cumulative plot and average values (insets) of spontaneous EPSC amplitude. The amplitude of spontaneous EPSC was unchanged by 10 μM CLZ in Scn1a+/− hippocampal CA1 slices. All data shown are means±s.e.m. from 15-20 recordings per treatment group.

FIGS. 38A-38H Reduced GABAergic neurotransmission in BTBR mice and enhancement by clonazepam. Spontaneous IPSC (sIPSC) and sEPSC were recorded in the hippocampal slices from 3-week old male BTBR and C57Bl/6J mice. Example traces of sIPSC (FIG. 38A) and cumulative plot and average values (inset) of sIPSC frequency in BTBR and C57BL/6J hippocampal CA1 neurons (FIG. 38B). Example traces of sEPSC (FIG. 38C) and cumulative plot and average values (inset) of sEPSC frequency in BTBR and C57BL/6J hippocampal CA1 neurons (FIG. 38D). Example traces of sIPSC (FIG. 38E) and cumulative plot and average value (inset) of sIPSC amplitude (FIG. 38F) in clonazepam and vehicle treated BTBR CA1 slices. Example traces of sEPSC (FIG. 38G) and cumulative plot and average value (inset) of sIPSC frequency (FIG. 38H) in clonazepam and vehicle treated BTBR CA1 slices. CON, Control. CLZ, Clonazepam. All data shown are means±s.e.m. from 15-19 recordings per strain. *P<0.05, **P<0.01, ***P<0.001.

FIGS. 39A-39M Effects of low-dose clonazepam on social interaction and cognitive deficits in BTBR mice. (FIGS. 39A-39D) Age-matched male C57BL/6J (n=6; each group) and BTBR mice (n=9; each group) were treated with a single intraperitoneal dose of clonazepam at the indicated level and subjected to the three-chamber social interaction test. Test mice were not reused; different groups of mice were used for each dose of clonazepam treatment. (FIGS. 39A, 39B) Time in chambers. (FIGS. 39C, 39D) Ratio of time in mouse chamber to time in object chamber. (FIGS. 39E-39G) The effects of low-dose clonazepam (CLZ; 0.05 mg/kg) on open field activity were measured in C57BL/6J (n=8) and BTBR mice (n=10). (FIG. 39H) Time in open arms in elevated plus maze for BTBR mice (n=10) and C57BL/6J mice (n=10). (FIG. 39I) In the three-chamber test, BTBR (blue) and C57BL/6J (black) mice were treated with vehicle (Pre, Post) or low-dose clonazepam (CLZ) 30 min before testing social interactions on Day 0 (Pre), Day 7 (CLZ), or Day 14 (Post). The ratio of interaction time with the stranger mouse or object is plotted. (FIGS. 39J-39K) In the open-field reciprocal social interaction test, BTBR and C57BL/6J mice were treated with vehicle (Pre, Post) or low-dose clonazepam (CLZ) 30 min before testing social interactions on Day 0 (Pre), Day 7 (CLZ), or Day 14 (Post). The ratio of interaction time with the stranger mouse vs. object is plotted for BTBR mice (n=9) or C57BL/6J mice (n=9) for total interaction time (FIG. 39J) and nose-to-nose interaction time (K). (FIGS. 39L-39M) To test tolerance to the effects of low-dose clonazepam on locomotor and social behaviors, BTBR mice (n=10; each group) were treated with low-dose (0.05 mg/kg), and high-dose (1 mg/kg) clonazepam for 14 days. (FIG. 39L) Total distance moved during the open field test after drug treatment on Day 1 and Day 14 was measured, and % activity change was calculated by comparing the activity on Day 1 and Day 14 of treatment with the indicated doses of clonazepam. (FIG. 39M) Social interaction behavior was compared after treatment with low-dose clonazepam (n=10 for each group). CON, Control. CLZ, Clonazepam. All data shown are means±s.e.m. *P<0.05, **P<0.01, ***P<0.001.

FIGS. 40A-40I Effects of low-dose clonazepam on context-dependent spatial learning and memory deficits in BTBR mice. (A and B) Increasing doses (0, 0.0125, 0.05, and 0.1 mg/kg) of clonazepam were administered 30 min prior to context-dependent fear conditioning. (A) C57BL/6J (n=5-7). (B) BTBR (n=5-7) (C and D) Barnes circular maze. Spatial learning was measured for C57BL/6J and BTBR mice (n=10 each) by measurement of the latency (C) and number of errors (D) for mice to find safety without and with treatment with 0.05 mg/kg clonazepam as indicated. Note that treatment with clonazepam significantly improved the performance of BTBR mice but, in contrast, significantly worsened the performance of C57BL/6J mice. (E-G) On Day 5 of the Barnes maze test, a single injection of low-dose clonazepam was given 30 min prior to the trial for BTBR and C57BL/6J mice: (E), latency to target; (F), % correct pokes; (G), % time in target. (H and I) BTBR mice (n=10) were treated with 0.05 mg/kg clonazepam for 14 days. Contextual fear conditioning was performed 30 min after injection on Day 1 (H) and Day 14 (I).CON, Control. CLZ, Clonazepam. All data shown are means±s.e.m. *P<0.05, **P<0.01, ***P<0.001.

FIGS. 41A-41O Effects of positive and negative GABA-A receptor allosteric modulators on social behaviors and cognitive deficit. (FIGS. 41A-41B) Effect of clobazam (0.05 mg/kg) on social interaction behavior of BTBR mice in the three-chamber test (n=7-8). (FIGS. 41C, 41D) Effect of clobazam (0.05 mg/kg) on BTBR mice (n=7-8) in the open field test on total distance moved (FIG. 41C) and time spent in center (FIG. 41D). (FIGS. 41E, 41F) Effect of DMCM (0.2 mg/kg) on C57BL/6J mice (n=7-8) in the three-chamber test. (FIGS. 41G, 41H) Effect of DMCM (0.2 mg/kg) on overall exploratory behavior of C57BL/6J mice (n=8) in the open field test, measured as distance moved (FIG. 41G). Anxiety-like behavior of C57BL/6J mice (n=8) in the elevated plus maze test, measured as time in the open arms (FIG. 41H). (FIGS. 41I, 41J) Social interaction behavior of BTBR mice (n=6-8) in the 3-chamber test following treatment with the indicated doses of L838417 (FIG. 41I) or zolpidem (FIG. 41J). Test mice were not reused; different groups of mice were used for each dose. (FIGS. 41K, 41L) Contextual fear conditioning test of BTBR mice (n=5) following treatment with 0.05 mg/kg of L-838,417 (FIG. 41K) or zolpidem (FIG. 41L). Control data were replotted from FIG. 39B. (FIG. 39M) Effect of DMCM (0.2 mg/kg) on 129SvJ mice (n=8) in the three-chamber test. (FIGS. 41N, 41O) Effects of L838,417 (FIG. 41N) and zolpidem (FIG. 41O) on Scn1a+/− mice in the three-chamber test. CON, Control. CBZ, Clobazam. CLZ, Clonazepam. All data shown are means±s.e.m. *P<0.05, **P<0.01, ***P<0.001.

FIGS. 42A-42L Effects of low-dose clonazepam treatment on GABAergic neurotransmission in BTBR and C57BL/6J mice. (FIG. 42A) Cumulative plot and average values (inset) of sIPSC amplitude was unchanged in BTBR hippocampal CA1 slices when compared to C57BL/6J (BL6) slices. (FIG. 42B) Cumulative plot and average values (inset) of sEPSC amplitude. The amplitude of sEPSC was significantly increased in BTBR hippocampal CA1 slices when compared to C57BL/6J slices. (FIG. 42C) Cumulative plot and average values (inset) of sIPSC frequency. The frequency of sIPSC was significantly increased by 0.5 μM clonazepam in BTBR hippocampal CA1 slices. (FIG. 42D) Cumulative plot and average values (inset) of sEPSC amplitude. The amplitude of sEPSC was unchanged by clonazepam in BTBR slices. (FIGS. 42E-42H) The amplitude and the frequency of miniature IPSC and miniature EPSC were measured in BTBR and C57BL/6J hippocampal CA1 slices. (FIGS. 42E, 42F) the frequency (FIG. 42E) and the amplitude (FIG. 42F) of miniature IPSC were unchanged in BTBR slices when compared to C57BL/6J slices. (FIGS. 42G, 42H) the frequency of miniature EPSC was unchanged (FIG. 42G), but the amplitude of miniature EPSC was significantly increased (FIG. 42H) in BTBR slices when compared to C57BL/6J slices. (FIG. 42I-L) The frequency and amplitude of spontaneous IPSC and spontaneous EPSC in C57BL/6J. (FIGS. 42I, 42J) the frequency (FIG. 42I) and the amplitude (FIG. 42J) of spontaneous IPSC were not changed by the bath application of 0.5 μM clonazepam in C57BL/6J hippocampal CA1 slices. (FIGS. 42K, 42L) the frequency of spontaneous EPSC was decreased (FIG. 42K), but the amplitude of spontaneous EPSC was not changed (FIG. 42L) by 0.55 μM clonazepam treatment in C57BL/6J hippocampal CA1 slices. CON, Control. CLZ, Clonazepam. All data shown are means±s.e.m. from 15-19 recordings per strain. #, P=0.054; *, P<0.05, **, P<0.01, ***, P<0.001

FIGS. 43A-43I Low-dose clonazepam rescues social interaction and cognitive deficits in BTBR mice. (FIGS. 43A, 43B) In the three-chamber social interaction test, the time spent in close interaction between the test mouse and the stranger mouse was measured. (FIG. 43A) Close interaction behavior of C57BL/6J mice (n=6; each group) was not altered by a single injection of clonazepam, dose range from 0-0.1 mg/kg. (FIG. 43B) Clonazepam treatment rescued the decreased closed interaction behavior in BTBR mice (n=9; each group) in a dose-dependent manner. Interestingly, the rescuing effect was not observed at higher than maximally effective concentration (0.05 mg/kg). (FIG. 43C-43F) In the three-chamber test, social deficits in BTBR mice (n=8) were reversibly rescued by treatment with low-dose clonazepam. The social interaction preference in C57BL/6J mice (n=7) was not changed by low-dose clonazepam, measured by time spent in the chamber (FIG. 43C), or by time spent in close interaction (FIG. 43D), but BTBR mice showed recovered social interaction behaviors after clonazepam treatment, measured by time spent in the chamber (FIG. 43E), or by time spent in close interaction (FIG. 43F), and the clonazepam effect disappeared after a 1-week clearance period in the same mice. (FIG. 43G) To test tolerance to the effects of clonazepam on locomotor and social behaviors, BTBR mice (n=10; each group) were treated with low-dose (0.05 mg/kg), and high-dose (1 mg/kg) clonazepam for 14 days. Total distance moved during open field test at Day 1 and at Day 14 was measured. Whereas chronic high-dose clonazepam treatment caused significantly increased locomotor activity after drug treatment on Day 14 compared to Day 1, chronic low-dose clonazepam did not cause any increase in locomotor activity after drug treatment on either day. (FIG. 43H, 43I) To monitor the sedative effect of clonazepam, total distance moved was measured during the three-chamber social interaction test in BTBR mice (n=9; each group) (FIG. 43H) and during the elevated plus maze test in BTBR mice (n=10) and C57BL/6J mice (n=10) (FIG. 43I). The clonazepam treatment did not cause sedation during the three-chamber test and the elevated plus maze test. CON, Control. CLZ, Clonazepam. All data shown are means±s.e.m. *P<0.05, **P<0.01, ***P<0.001.

FIGS. 44A-44D Total distance moved during contextual fear conditioning. (FIGS. 44A-44D Total distance moved was measured during contextual fear conditioning, which is inversely correlated with freezing behavior. CON, Control. CLZ, Clonazepam. All data shown are means±s.e.m. *P<0.05, **P<0.01, ***P<0.001.

FIGS. 45A-45J Rescue of social and cognitive deficits in BTBR mice by subunit-specific GABA-A receptor positive allosteric modulators. (FIGS. 45A, 45B) In the 3-chamber test, 0.2 mg/kg DMCM impaired the normal close interaction behavior in C57BL/6J mice (n=8). (FIG. 45C) In the elevated plus maze test, the number of entries in the open arms was unchanged in C57BL/6J mice (n=7-8) by 0.2 mg/kg DMCM treatment. (FIG. 45D) In the open field test, the time spent in center was unaltered in C57BL/6J mice (n=8) by 0.2 mg/kg (FIGS. 45E, 45F) In the 3-chamber test, impaired social behavior, measured by the time spent in close interaction between the test mouse and the stranger mouse, was rescued at the doses of 0.05 mg/kg L-838,417, but the rescuing effect was disappeared at the dose of 0.5 mg/kg (FIG. 45E). Zolpidem failed to rescue social deficit in BTBR mice (n=6-8; each group), rather it made social behavior worse at the dose of 0.5 mg/kg (FIG. 45F). (FIG. 45G, 45H) The total distance moved during the 3-chamber test, was unchanged by L-838,417 with the dose range from 0 to 0.5 mg/kg (FIG. 45G), whereas the total distance moved during the 3-chamber test was dose-dependently decreased by zolpidem with the dose range from 0 to 0.5 mg/kg (FIG. 45H). (FIG. 45I, 45J) In the 3-chamber test, the social behavior was not changed by L-838,417 in C57BL/6J mice (FIG. 45I). Zolpidem dose-dependently impaired the normal social behaviors in C57BL/6J mice (n=7) (FIG. 45J). Test mice were not reused, different group of mice were used for each dose of drug treatments. CON, Control. All data shown are means±s.e.m. *P<0.05, **P<0.01, ***P<0.001.

### DETAILED DESCRIPTION

The methods and formulations described herein are based, in part, on the discovery that low doses of agents that increase the response of the GAB A-A receptor to GABA can be used to treat autism spectrum disorders or symptoms thereof. The effective dose of such agents, which can include, but are not limited to positive allosteric modulators of the GAB A-A receptor, including, but not limited to those that act at the benzodiazepine site of the GABA-A receptor, is well below that which results in substantial sedative, anxiolytic or anti-convulsive effects for which such agents are commonly prescribed or administered.

### DEFINITIONS

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used herein and unless otherwise indicated, the terms “a” and “an” are taken to mean “one”, “at least one” or “one or more”. Unless otherwise required by context, singular terms used herein shall include pluralities and plural terms shall include the singular.

As used herein, the term “autism spectrum disorder (ASD)” refers to a heterogeneous group of neurodevelopmental disorders as classified in the fifth revision of the American Psychiatric Association's Diagnostic and Statistical Manual of Mental Disorders 5th edition (DSM-5). The DSM-5 redefined the autism spectrum to encompass the previous (DSM-IV-TR) diagnoses of autism, Asperger syndrome, pervasive developmental disorder not otherwise specified (PDD-NOS), childhood disintegrative disorder, and Rett syndrome. The autism spectrum disorders are characterized by social deficits and communication difficulties, stereotyped or repetitive behaviors and interests, and in some cases, cognitive delays. For example, an ASD is defined in the DSM-5 as exhibiting (i) deficits in social communication and interaction not caused by general developmental delays (must exhibit three criteria including deficits in social-emotional reciprocity, deficits in nonverbal communication, and deficits in creating and maintaining relationships appropriate to developmental level), (ii) demonstration of restricted and repetitive patterns of behavior, interest or activities (must exhibit two of the following four criteria: repetitive speech, repetitive motor movements or repetitive use of objects, adherence to routines, ritualized patterns of verbal or nonverbal behavior, or strong resistance to change, fixated interests that are abnormally intense of focus, and over or under reactivity to sensory input or abnormal interest in sensory aspects of environment), (iii) symptoms must be present in early childhood, and (iv) symptoms collectively limit and hinder everyday functioning The term “ASD” is also contemplated herein to include Dravet's syndrome and autistic-like behavior in non-human animals. For the purposes of this disclosure, the treatment of Autism Spectrum disorders, autism-like behavior or symptoms are referred to collectively as the treatment of “autism” herein. That is, wherever treatment of “autism” is discussed, it should be interpreted to apply to the treatment of any disorder on the spectrum of Autism Spectrum Disorders or autism-like disorders or behavior.

As used herein with respect to benzodiazepine and non-benzodiazepine drugs that increase the response of the GABA-A receptor to GABA, the term “low-dose” refers to a dose that does not provide a therapeutically effective sedative, anxiolytic or anti-convulsive effect. In one embodiment, the dose is less than 40% of the dose of the same agent that achieves one or more of an anti-anxiety, anti-convulsive, or sedative effect. In other embodiments, the dose is less than 30% of the dose effective to achieve an anti-anxiety, anti-convulsive or sedative effect i.e., less than 20%, less than 10%, less than 5%, less than 2% or less than 1% of the dose necessary to achieve one or more of an anti-anxiety, anti-convulsive, or sedative effect. In other embodiments, the dose is less than 40%, 30%, 20%, 10%, 5%, 2% or 1% of the USP dose of the agent to achieve a sedative, anxiolytic or anti-convulsive effect.

As used herein, the term “agent that increases GABAergic signaling” refers to an agent that increases opening of the GABA receptor chloride channel thereby inducing hyperpolarization of the post-synaptic neuron. In some embodiments, the agent that increases GABAergic signaling is a GABA-A receptor agonist. In other embodiments, the agent that increases GABAergic signaling is a positive allosteric modulator of the GABA-A receptor.

As used herein, the term “positive allosteric modulator” refers to an agent that binds a receptor (e.g., the GABA-A receptor), at a site other than that bound by the natural ligand (e.g., GABA) and enhances the activity of the receptor in response to the natural ligand e.g., GABA, for example, by increasing the frequency of opening of the GABA chloride channel. A positive allosteric modulator need not activate the chloride channel of the GABA receptor directly (e.g., does not substitute for the neurotransmitter GABA), but rather allosterically enhances the effects of GABA at the receptor level.

As used herein, the term “efficacy at the α2 and/or α3 subunits of the GABA-A receptor” refers to the effect of a positive allosteric modulator of GABA-A receptor activity, wherein the agent binds to a site independent of the GABA site on a GABA-A receptor comprising α2 and/or α3 subunits and allosterically enhances the effects of GABA at the receptor. As an example, it is known in the art that benzodiazepines do not substitute for GABA to directly activate GAB A-A receptors nor do they facilitate opening of the chloride channel of the GABA-A receptor in the absence of GABA. Rather, agents active at the benzodiazepine site enhance the action of GAB A, particularly through increasing the frequency of the opening of the chloride channel. The term “efficacy” is used to differentiate from agents that have affinity for an allosteric site of the GABA-A receptor (e.g., are capable of binding) but do not enhance the opening of the chloride channel (e.g., do not have efficacy at that site). Such agents that do not have efficacy as a positive allosteric modulator of GABA-A, as that term is used herein, can act as antagonists or inverse agonists at e.g., the benzodiazepine site, however such activity is not encompassed by the term “efficacy at the α2 and/or α3 subunits of the GABA-A receptor” as used herein.

As used herein, the term “selective for the α2 and/or α3 subunits of the GABA-A receptor” refers to an agent that has efficacy as a positive allosteric modulator at the α2 and/or α3 subunits of the GABA-A receptor at a dose lower than the dose in which the agent has efficacy for GABA receptors comprising α1, α4, α5, or α6 subunits but not α2 and/or α3 subunits; for example, a positive allosteric modulator of the GABA-A receptor is “selective” for α2 and/or α3 subunits if the dose required to act as a positive allosteric modulator at one or more α1, α4, α5, or α6 subunits is at least 50% higher, at least 75% higher, at least 1-fold higher, at least 2-fold higher, at least 5-fold higher, at least 10-fold higher, at least 50-fold higher, at least 100-fold higher, at least 1000-fold higher or more than the dose required to act as a positive allosteric modulator at the α2 and/or α3 subunits. It is also specifically contemplated herein that a positive allosteric modulator at the α2 and/or α3 subunits can also act as an antagonist or inverse agonist at one or more α1, α4, α5, or α6 subunits. In one embodiment, the agent “selective for GABA receptors having α2 and/or α3 subunits” does not act as a selective positive allosteric modulator of GABA receptors having α1 or α5 subunits, but not α2 and/or α3 subunits.

As used herein, the term “partial agonist” is used to refer to an agent that produces a lower maximal response at full receptor occupancy than an agent considered to be a “full agonist.” A partial agonist is not defined by a lower affinity for a binding site, but rather a “partial agonist” is defined by its ability to competitively inhibit the responses produced by a full agonist at higher concentrations (e.g., to reduce the maximal response to that of the partial agonist). One of skill in the art of pharmacology or medicine will understand the use of the terms “partial agonist” and “full agonist” as used in the art. As used herein, the term “agonist” encompasses both full and partial agonists, but does not encompass inverse agonists e.g., at the benzodiazepine site.

As used herein, a “short acting benzodiazepine” will typically have a half-life of approximately 1-12 hours, and may or may not have active metabolites upon metabolism of the parent drug (e.g., triazolam; t1/2 of 2-3 hours). An “intermediate-acting benzodiazepine” will typically have a half-life of 12-40 hours, while a “long-acting benzodiazepine” will have a half-life of 40-250 hours. Often the intermediate- or long-acting benzodiazepines are metabolized to active metabolites with long half-lives that exhibit efficacy at the benzodiazepine site. For example, diazepam is metabolized to desmethyldiazepam, which acts as a benzodiazepine agonist and has a half-life of greater than 40 hours.

### Autism Spectrum Disorders

Autism Spectrum disorders (ASD) are a heterogeneous group of neurodevelopmental disorders that manifest during early childhood and are characterized by a range of stereotyped interests and impairments in social interaction and communication. Recent epidemiological studies have suggested that ASD is diagnosed in approximately 1% of children (Kogan et al., Pediatrics, 124(5): 1395-1403 (2009)).

Autistic disorders are characterized by marked impairment in communication and reciprocal social interaction, social skills, verbal communication, behavior, and cognitive function (Rapin, I. New Engl. J. Med., 337: 97-104 (1997); Lord, C. et al., Neuron, 28, 355-363 (2000)). Abnormalities in language development, mental retardation, and epilepsy are frequent problems in the clinical profile of patients with autism. The core symptoms of autism include abnormal communication, social relatedness, behavior, and cognition (Rapin, I., N. Engl. J. Med., 337: 97-104 (1997) and Lord, C. et al., Neuron, 28, 355-363 (2000)).

The majority of autistic children show abnormalities during infant development that may not become apparent until the second year of life. Approximately 30-50% of autistic children undergo regression, with a loss of skills, including language, between 16 and 25 months of age. In the medical evaluation of autism, specific etiologies can be found in <10% of children, including fragile X, tuberous sclerosis, and other rare diseases (Cohen, D. et al., Journal of Autism & Developmental Disorders, 35, 103-116 (2005)). Epilepsy occurs in up to 40% of patients, and epileptic discharges may occur on EEGs early in childhood, even in the absence of clinical seizures (Tuchman, R. and Rapin, I., Lancet Neurology, 1, 352-358 (2002)).

### GABA-A Receptor—General

GABA-A receptors are ligand-gated ion channels that belong to the same super family of receptors as glycine, nicotinic cholinergic, and serotonin 5HT-3 receptors.

The GABA-A receptor complex is a pentameric receptor protein structure formed by co-assembly of subunits from seven different classes. Five subunits are situated in a circular array surrounding a central chloride-permeable pore. The particular combination of subunits yields receptors with different pharmacological and physiological properties.

### GABAergic Signaling in Autism Spectrum Disorders

Autism spectrum disorders (ASD) are developmental neuropsychiatric diseases with characteristic symptoms of impaired social interaction, stereotyped behaviors, and delayed language development (Abrahams and Geschwind, 2008; Geschwind, 2011). One hypothesis is that the core behavioral features of autism are caused by an imbalance between excitatory and inhibitory neurotransmission in the brain (Gatto and Broadie, 2010; Markram and Markram, 2010; Rubenstein and Merzenich, 2003). Recent work on mouse models of syndromic autism caused by monogenic mutations in MeCP2, Scn1a, Shank3, and Cntnap2 has shown that an increased ratio of excitatory to inhibitory neurotransmission in the brain may cause autistic-like behaviors (Auerbach et al., 2011; Chao et al., 2010; Han et al., 2012; Peca et al., 2011; Penagarikano et al., 2011), and optogenetic increase in excitation/inhibition ratio can also induce social interaction deficits (Yizhar et al., 2011). The increased ratio of excitatory to inhibitory neurotransmission in these models may arise by increased excitatory transmission, decreased inhibitory transmission, or both.

This emerging research implicates increased excitation to inhibition ratio in causing autistic-like behaviors in monogenic animal models of autism, but there is much less evidence for the significance of this mechanism in idiopathic models of autism. BTBR mice are a well-studied model of idiopathic autism (Defensor et al., 2011; McFarlane et al., 2008; Yang et al., 2012). However, the inherited genetic changes that led to autistic-like behaviors in these mice are incompletely known and still under active investigation (Jones-Davis et al., 2013). In the Examples section, the inventors provide evidence from recordings of spontaneous synaptic transmission that BTBR mice have a reduced level of inhibitory neurotransmission mediated by GABA-A receptors in the hippocampus compared to the control strain C57BL/6J, which may contribute to their autistic-like behaviors.

Activation of GABA-A receptors by GABA is enhanced by benzodiazepines, which are used in treatment of epilepsy, anxiety, panic disorder, and insomnia (Rudolph and Knoflach, 2011). Moreover, genetic linkage of the GAB A-A receptor to autism has been widely reported (Li et al., 2012). However, GABA-A receptors have not been recognized as a therapeutic target for ASDs because of their sedative activity. Data shown in the Examples section indicates low-dose clonazepam is effective in treatment of impaired social interaction and cognitive deficit in Scn1a+/− mice, a model of Dravet Syndrome with marked autistic-like behaviors (Han et al., 2012). Evidence is also presented in the Examples section indicating that treatment with low doses of positive allosteric benzodiazepine modulators of GABA-A receptors improves characteristic autistic-like behaviors in BTBR mice. Interestingly, negative allosteric modulation of GABA-A receptors with benzodiazepines induces social interaction deficits in C57BL/6J and 129SvJ wild type (WT) mice, supporting a causal role for reduced inhibitory neurotransmission in some features of autism. Moreover, autistic-like behavioral impairments can be treated effectively in both BTBR and Scn1a+/− mice by enhancement of inhibitory neurotransmission with low doses of subunit-selective positive allosteric modulators of GABA-A receptors containing α2 and/or α3 subunits. Together, the results shown in the Examples section indicate that reduced GABAergic inhibitory neurotransmission contributes to autism-associated behavioral and cognitive deficits and indicates that enhancement of GABAergic neurotransmission with subunit-specific pharmacological agents can be beneficial.

### Benzodiazepines

The benzodiazepine class of drugs possesses sedative, tranquilizing and muscle relaxing properties. These agents are frequently classified as anxiolytic, anticonvulsives, sedatives, and skeletal muscle relaxants. Benzodiazepines are useful in preventing, treating, or ameliorating the symptoms of anxiety, insomnia, agitation, seizures (such as those caused by epilepsy), muscle spasms and rigidity, the symptoms of drug withdrawal associated with the continuous abuse of central nervous system depressants, and exposure to nerve agents.

Benzodiazepines act by binding to the GABA-A receptor of a neuron, possibly causing the receptor to change shape and making it more accessible to gamma-aminobutyric acid (GABA). GABA is an inhibitory neurotransmitter that, when bound to the GABA-A receptor facilitates entry of chloride ions into the neuron. The increase in chloride ions hyperpolarizes the membrane of the neuron. This completely or substantially reduces the ability of the neuron to generate an action potential. Targeting the GAB A-A receptor is particularly useful in treating many disorders, such as tetanus and epilepsy, which may result from too many action potentials proceeding through the nervous system.

Drugs in the benzodiazepine family, particularly those that are full agonists or partial agonists at the GABA-A receptor, can be used with the methods and assays described herein. Benzodiazepines that act as antagonists or inverse agonists are not contemplated for use as described herein. Exemplary benzodiazepines active at the GABA-A receptor are listed herein in Table 1. Also contemplated for the methods and formulations described herein are active metabolites of the benzodiazepine drugs listed in Table 1.

In some embodiments, the benzodiazepine drug is clonazepam, clobazam, or a pharmaceutically acceptable salt thereof. In some embodiments, the benzodiazepine drug is a full agonist at the GAB A-A receptor. In other embodiments, the benzodiazepine drug is a partial agonist at the GAB A-A receptor. In some embodiments, the benzodiazepine drug is a short-acting benzodiazepine. In other embodiments, the benzodiazepine drug is a long-acting benzodiazepine. In some embodiments, the benzodiazepine drug is a typical benzodiazepine. In other embodiments, the benzodiazepine drug can be an atypical benzodiazepine.

In some embodiments, the benzodiazepine drug acts on the α2,3 subtype of the GABA-A receptor (e.g., has efficacy at the α2 and/or α3 subunits). In one embodiment, the benzodiazepine drug is selective for the α2 and/or α3 subunits of the GABA-A receptor (e.g., pyrazolam).

In some embodiments, the benzodiazepine drug is a prodrug, although active metabolites of such prodrugs are also contemplated for use as described herein.

TABLE 1
List of Exemplary Benzodiazepines

| | | Dose in humans | |
|---|---|---|---|
| | | for anti-anxiety, | |
| | Effect at | anti-convulsive, | |
| | GABA-A | or sedative | |
| Drug | receptor | effects | Structure |
| Adinazolam (DERACYN ™) | Full agonist | N/A | 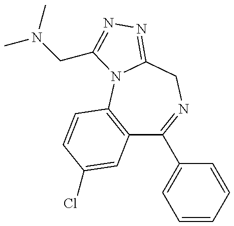 [SMILES: CN(C)Cc1nnc2n1-c1ccc(Cl)cc1C(c1ccccc1)=NC2] |
| Alprazolam (XANAX ™, HELEX ™, XANOR ™, ONAX ™, ALPROX ™, RESTYL ™, TAFIL ™, PAXAL ™) | Full agonist | dose range of 0.5 to 3.0 mg | 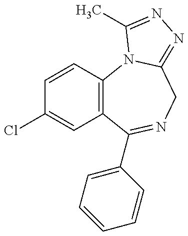 [SMILES: Cc1nnc2n1-c1ccc(Cl)cc1C(c1ccccc1)=NC2] |
| Arfendazam | Partial agonist | N/A | 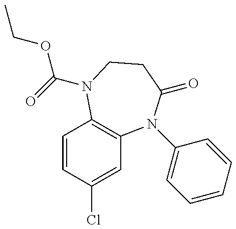 [SMILES: CCOC(=O)N1CCC(=O)N(c2ccccc2)c2cc(Cl)ccc21] |
| Avizafone | Full agonist | 3.5 mg/kg | 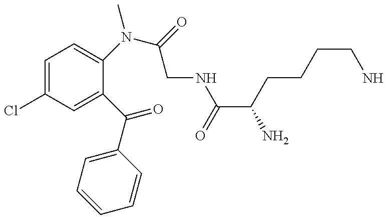 [SMILES: CN(C(=O)CNC(=O)[C@@H](N)CCCCN)c1ccc(Cl)cc1C(=O)c1ccccc1] |
| Bentazepam (THIADIPONE ™) | Agonist | N/A | 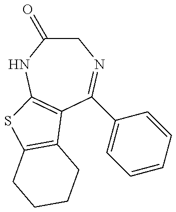 [SMILES: O=C1CN=C(c2ccccc2)c2c(sc3c2CCCC3)N1] |
| Bretazenil | Partial Agonist High potency | 0.5 mg* | 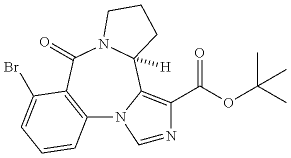 [SMILES: CC(C)(C)OC(=O)c1ncn2c1[C@@H]1CCCN1C(=O)c1c(Br)cccc1-2] |
| Bromazepam (LECTOPAM ™, LEXOTAN ™, LEXILIUM ™, LEXAURIN ™, BRAZEPAM ™, REKOTNI ™L, BROMAZE ™ and LEXOTANIL ™) | Agonist | 5-6 mg* | 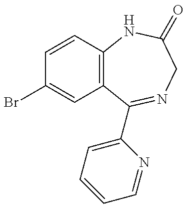 [SMILES: O=C1CN=C(c2ccccn2)c2cc(Br)ccc2N1] |
| Brotizolam (LENDORMIN ™, DORMEX ™, SINTONAL ™, NOCTILAN ™) | Agonist | 0.25 mg* (anti-anxiety 80- 100 mg Sedative 0.125- 25 mg) | 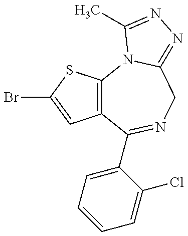 [SMILES: Cc1nnc2n1-c1sc(Br)cc1C(c1ccccc1Cl)=NC2] |
| Camazepam (ALBEGO ™, LIMPIDON ™, PAXOR ™) | Agonist | N/A | 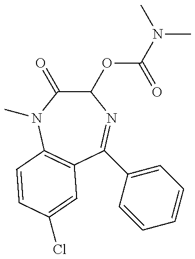 |
| Chlordiazepoxide (LIBRIUM ™, RISOLID ™, ELENIUM ™) | Agonist Long- acting | 25 mg* | 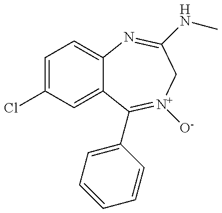 [SMILES: CNC1=Nc2ccc(Cl)cc2C(c2ccccc2)=[N+]([O-])C1] |
| Ciclotizolam | Partial Agonist Low efficacy | N/A | 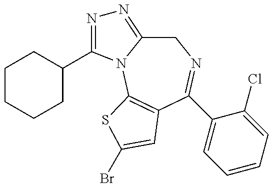 [SMILES: Clc1ccccc1C1=NCc2nnc(C3CCCCC3)n2-c2sc(Br)cc21] |
| Cinolazepam (GERODORM ™) | Agonist | 40 mg* | 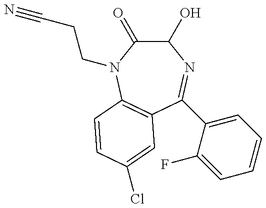 [SMILES: N#CCCN1C(=O)C(O)N=C(c2ccccc2F)c2cc(Cl)ccc21] |
| Clazolam | Agonist | N/A | 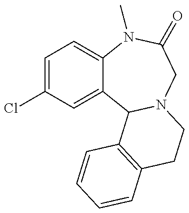 [SMILES: CN1C(=O)CN2CCc3ccccc3C2c2cc(Cl)ccc21] |
| Climazolam | Agonist | N/A | 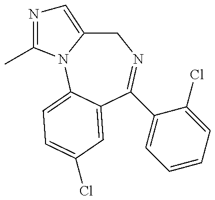 [SMILES: Cc1ncc2n1-c1ccc(Cl)cc1C(c1ccccc1Cl)=NC2] |
| Clobazam (FRISIUM ™, URBANOL ™, ONFI ™) | Agonist | 20 mg* | 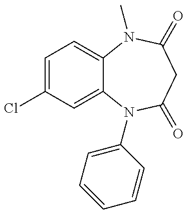 [SMILES: CN1C(=O)CC(=O)N(c2ccccc2)c2cc(Cl)ccc21] |
| Clonazepam (RIVATRIL ™, RIVOTRI ™L, KLONOPIN, ™ IKTORIVIL ™, PAXAM ™) | Agonist Long- acting | 0.5 mg* | 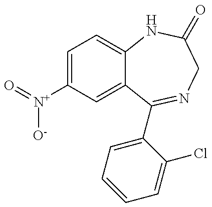 [SMILES: O=C1CN=C(c2ccccc2Cl)c2cc([N+](=O)[O-])ccc2N1] |
| Clorazepate (TRANXENE ™, TRANXILIUM ™) | Partial Agonist (Prodrug) | 15 mg* 15-60 mg/day | 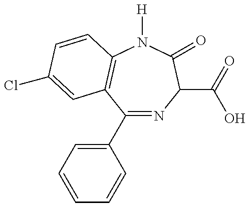 [SMILES: O=C(O)C1N=C(c2ccccc2)c2cc(Cl)ccc2NC1=O] |
| Clotiazepam (VERATRAN ™, CLOZAN ™, RIZE ™) | Full agonist | 5-10 mg* | 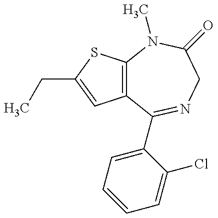 [SMILES: CCc1cc2c(s1)N(C)C(=O)CN=C2c1ccccc1Cl] |
| Cloxazolam (SEPAZON ™, OLCADIL ™) | Agonist (Prodrug) | 1 mg* | 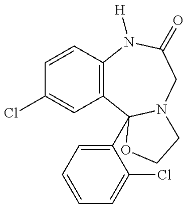 [SMILES: O=C1CN2CCOC2(c2ccccc2Cl)c2cc(Cl)ccc2N1] |
| Cyprazepam | Agonist | N/A | 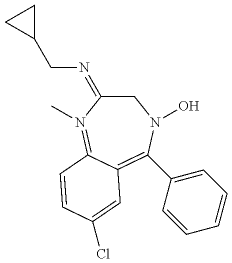 |
| Delorazepam (DADUMIR ™) | Agonist | 1 mg* | 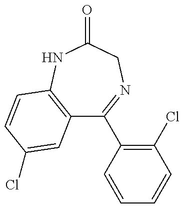 [SMILES: O=C1CN=C(c2ccccc2Cl)c2cc(Cl)ccc2N1] |
| Diazepam (VALIUM ™) | Agonist Long- acting | 10 mg* Typical dose: 2 mg to 10 mg 2-4 times daily Pediatric patients: 1 mg- 2.5 mg 3-4 × daily to start | 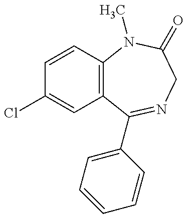 [SMILES: CN1C(=O)CN=C(c2ccccc2)c2cc(Cl)ccc21] |
| Doxefazepam | Agonist | N/A | 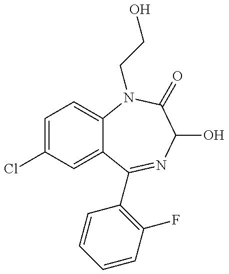 [SMILES: O=C1C(O)N=C(c2ccccc2F)c2cc(Cl)ccc2N1CCO] |
| Elfazepam | Agonist | N/A | 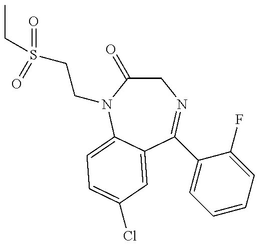 [SMILES: CCS(=O)(=O)CCN1C(=O)CN=C(c2ccccc2F)c2cc(Cl)ccc21] |
| Estazolam (PROSOM ™) | Agonist | 2 mg* | 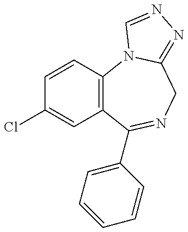 [SMILES: Clc1ccc2c(c1)C(c1ccccc1)=NCc1nncn1-2] |
| Ethyl carfluzepate | Agonist | N/A | 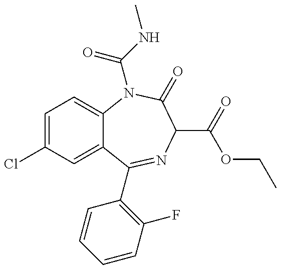 [SMILES: CCOC(=O)C1N=C(c2ccccc2F)c2cc(Cl)ccc2N(C(=O)NC)C1=O] |
| Ethyl dirazepate | Agonist | N/A | 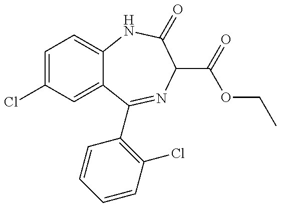 [SMILES: CCOC(=O)C1N=C(c2ccccc2Cl)c2cc(Cl)ccc2NC1=O] |
| Ethyl loflazepate (VICTAN ™, MEILAX ™, RONLAX ™) | Agonist (Prodrug) | 2 mg* | 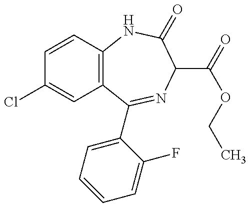 [SMILES: CCOC(=O)C1N=C(c2ccccc2F)c2cc(Cl)ccc2NC1=O] |
| Etizolam (ETILAAM ™, PASADEN ™, DEPAS ™) | Full agonist | 0.5-1 mg* 0.5 mg-3 mg/day | 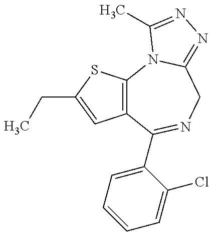 [SMILES: CCc1cc2c(s1)-n1c(C)nnc1CN=C2c1ccccc1Cl] |
| Fletazepam | Agonist | N/A | 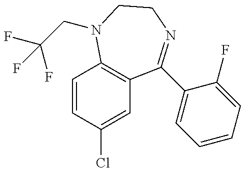 [SMILES: Fc1ccccc1C1=NCCN(CC(F)(F)F)c2ccc(Cl)cc21] |
| Fludiazepam | Agonist | N/A | 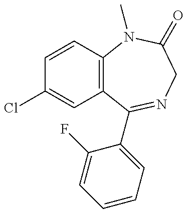 [SMILES: CN1C(=O)CN=C(c2ccccc2F)c2cc(Cl)ccc21] |
| Flunitrazepam (ROHYPNOL ™, HIPNOSEDON ™, VULBEGAL ™, FLUSCAND ™, FLUNIPAM ™, RONAL ™, ROHYDORM ™) | Agonist | 1 mg* | 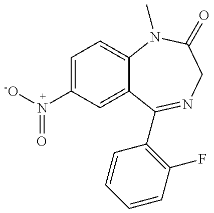 |
| Flurazepam (DALMADORM ™, DALMANE ™) | Partial agonist | 15-30 mg* | 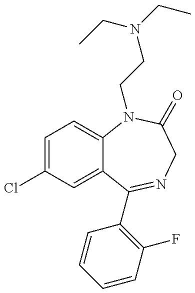 [SMILES: CCN(CC)CCN1C(=O)CN=C(c2ccccc2F)c2cc(Cl)ccc21] |
| Flutazolam | Agonist | N/A | 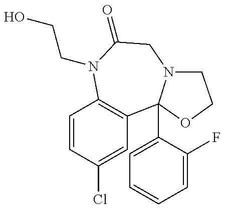 [SMILES: O=C1CN2CCOC2(c2ccccc2F)c2cc(Cl)ccc2N1CCO] |
| Flutemazepam | Agonist | N/A | 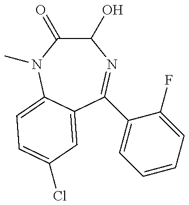 [SMILES: CN1C(=O)C(O)N=C(c2ccccc2F)c2cc(Cl)ccc21] |
| Flutoprazepam (RESTAS ™) | Agonist | 2-3 mg* | 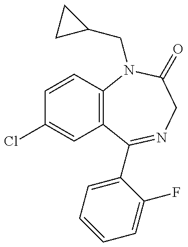 [SMILES: O=C1CN=C(c2ccccc2F)c2cc(Cl)ccc2N1CC1CC1] |
| Fosazepam | Agonist Low potency | 60 mg* | 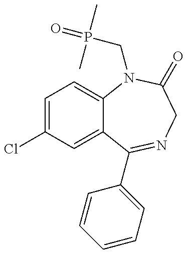 [SMILES: CP(C)(=O)CN1C(=O)CN=C(c2ccccc2)c2cc(Cl)ccc21] |
| Gidazepam | Agonist (Prodrug) | N/A | 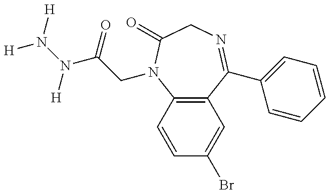 [SMILES: NNC(=O)CN1C(=O)CN=C(c2ccccc2)c2cc(Br)ccc21] |
| Girisopam | Agonist | N/A | 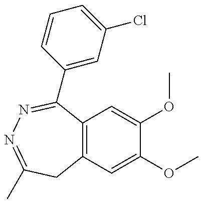 |
| Halazepam (ALAPRYL ™, PACINONE ™) | Agonist | 20-40 mg* | 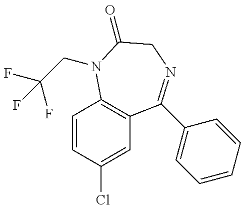 [SMILES: O=C1CN=C(c2ccccc2)c2cc(Cl)ccc2N1CC(F)(F)F] |
| Haloxazolam (SOMELIN ™) | Agonist | N/A | 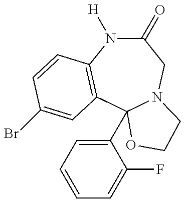 [SMILES: O=C1CN2CCOC2(c2ccccc2F)c2cc(Br)ccc2N1] |
| Iclazepam | Agonist | N/A | 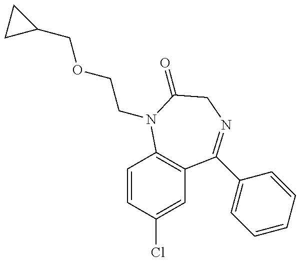 [SMILES: O=C1CN=C(c2ccccc2)c2cc(Cl)ccc2N1CCOCC1CC1] |
| Imidazenil | Partial agonist | N/A | 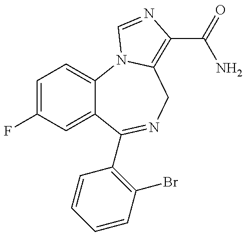 [SMILES: NC(=O)c1ncn2c1CN=C(c1ccccc1Br)c1cc(F)ccc1-2] |
| Ketazolam (ANSEREN ™, ANSIETEN ™, ANSIETIL ™, MARCEN ™, SEDATIVAL ™, SEDOTIME ™, SOLATRAN ™ and UNAKALM ™) | Agonist | 15-30 mg* | 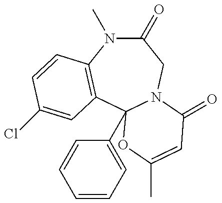 [SMILES: CC1=CC(=O)N2CC(=O)N(C)c3ccc(Cl)cc3C2(c2ccccc2)O1] |
| Lofendazam | Agonist | N/A | 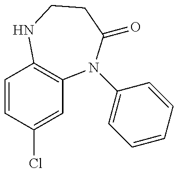 [SMILES: O=C1CCNc2ccc(Cl)cc2N1c1ccccc1] |
| Lopirazepam | Agonist Short- acting | N/A | 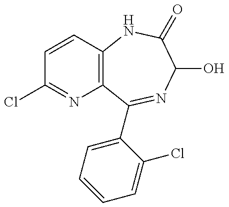 [SMILES: O=C1Nc2ccc(Cl)nc2C(c2ccccc2Cl)=NC1O] |
| Loprazolam (DORMONOCT ™) | Agonist | 2 mg* 1-2 mg | 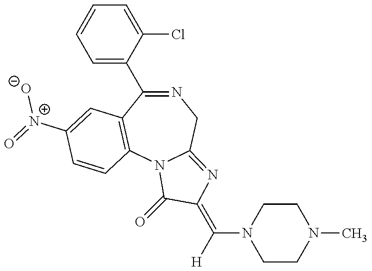 [SMILES: CN1CCN(/C=C2\N=C3CN=C(c4ccccc4Cl)c4cc([N+](=O)[O-])ccc4N3C2=O)CC1] |
| Lorazepam (ATIVAN ™, LORENIN ™, LORSILAN ™, TEMESTA ™, TAVOR ™, LORABENZ ™) | Agonist High- potency | 1 mg* | 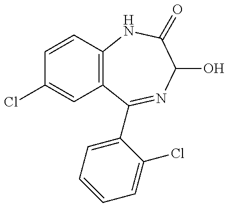 [SMILES: O=C1Nc2ccc(Cl)cc2C(c2ccccc2Cl)=NC1O] |
| Lormetazepam (LORAMET ™, NOCTAMID ™, PRONOCTAN ™) | Agonist Short- acting | 1.5 mg* | 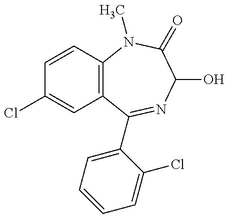 [SMILES: CN1C(=O)C(O)N=C(c2ccccc2Cl)c2cc(Cl)ccc21] |
| Meclonazepam | Agonist | N/A | 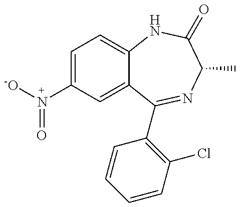 |
| Medazepam (NOBRIUM ™, RUDOTEL ™, RAPORAN ™, ANSILAN ™ and MEZAPAM ™) | Agonist Long- acting | 10 mg* | 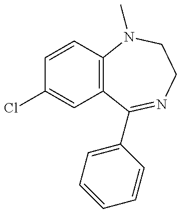 [SMILES: CN1CCN=C(c2ccccc2)c2cc(Cl)ccc21] |
| Menitrazepam | Agonist | N/A | 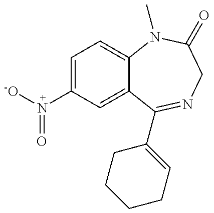 |
| Metaclazepam (TALIS ™) | Agonist | N/A | 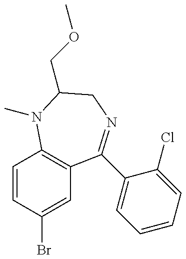 [SMILES: COCC1CN=C(c2ccccc2Cl)c2cc(Br)ccc2N1C] |
| Mexazolam (MELEX ™, SEDOXIL ™) | Agonist | N/A | 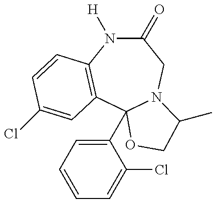 [SMILES: CC1COC2(c3ccccc3Cl)c3cc(Cl)ccc3NC(=O)CN12] |
| Midazolam (DORMICUM ™, VERSED ™, HYPNOVEL ™, DORMONID ™) | Agonist Short- acting | 7.5 mg* | 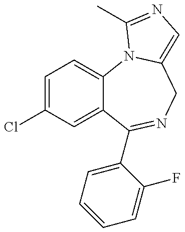 [SMILES: Cc1ncc2n1-c1ccc(Cl)cc1C(c1ccccc1F)=NC2] |
| Nerisopam | Agonist | N/A | 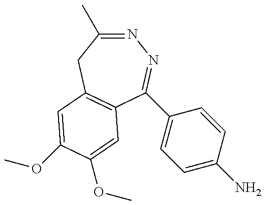 [SMILES: COc1cc2c(cc1OC)C(c1ccc(N)cc1)=NN=C(C)C2] |
| Nimetazepam (ERIMIN ™) | Agonist | 5 mg* | 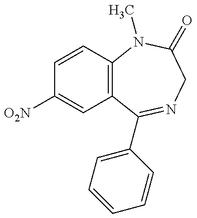 [SMILES: CN1C(=O)CN=C(c2ccccc2)c2cc([N+](=O)[O-])ccc21] |
| Nitrazepam (MOGADON ™, ALODORM ™, PACISYN ™, DUMOLID ™, NITRAZADON ™) | Full agonist Long- acting | 10 mg* | 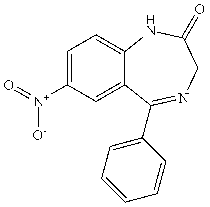 [SMILES: O=C1CN=C(c2ccccc2)c2cc([N+](=O)[O-])ccc2N1] |
| Nitrazepate | Agonist | N/A | 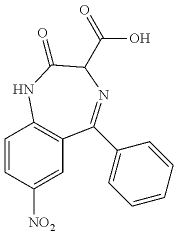 [SMILES: O=C(O)C1N=C(c2ccccc2)c2cc([N+](=O)[O-])ccc2NC1=O] |
| Nordazepam (NORDAZ ™, MADAR ™, STILNY ™, VEGESAN ™, CALMDAY ™) | Partial agonist | 10 mg* | 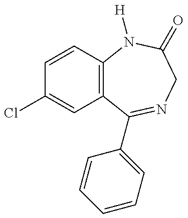 [SMILES: O=C1CN=C(c2ccccc2)c2cc(Cl)ccc2N1] |
| Oxazepam (SERESTA ™, SERAX ™, SERENID ™, SEREPAX ™, SOBRIL ™, OXABENZ ™, OXAPAX ™, OPAMOX ™) | Agonist Short- acting | 20 mg* Mild/moderate anxiety - 10 to 15 mg, 1 to 2 times daily Severe anxiety - 15 to 30 mg, 3 to 4 times daily Symptoms related to alcohol withdrawal - 15 to 30 mg, 3 to 4 times daily | 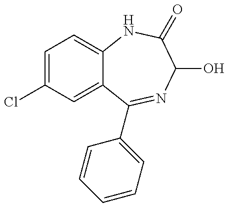 [SMILES: O=C1Nc2ccc(Cl)cc2C(c2ccccc2)=NC1O] |
| Oxazolam | Agonist | N/A | 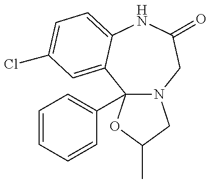 [SMILES: CC1CN2CC(=O)Nc3ccc(Cl)cc3C2(c2ccccc2)O1] |
| Phenazepam | Agonist | 1 mg* ave. dose: 0.5 mg 2-3 × daily (daily dose not to exceed 10 mg) | 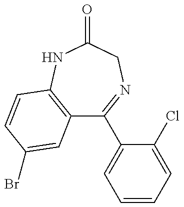 [SMILES: O=C1CN=C(c2ccccc2Cl)c2cc(Br)ccc2N1] |
| Pinazepam (DOMAR ™) | Agonist | 20 mg* | 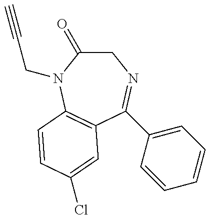 [SMILES: C#CCN1C(=O)CN=C(c2ccccc2)c2cc(Cl)ccc21] |
| Pivoxazepam | Agonist | N/A | 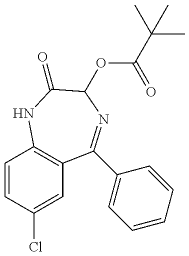 [SMILES: CC(C)(C)C(=O)OC1N=C(c2ccccc2)c2cc(Cl)ccc2NC1=O] |
| Prazepam (CENTRAC ™, CENTRAX ™, DEMETRIN ™, LYSANXIA ™, MONODEMETRIN ™, POZAPAM ™, PRASEPINE ™, PRAZENE ™, REAPAM ™ and TREPIDAN ™) | Agonist (Prodrug) | 20 mg* | 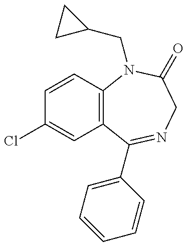 [SMILES: O=C1CN=C(c2ccccc2)c2cc(Cl)ccc2N1CC1CC1] |
| Premazepam | Partial agonist | 15 mg* | 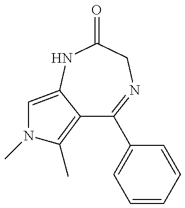 [SMILES: Cc1c2c(cn1C)NC(=O)CN=C2c1ccccc1] |
| Proflazepam (Ro10-3580) | Agonist | N/A | 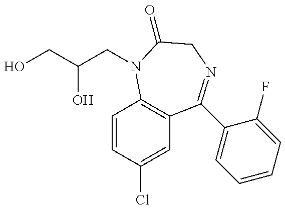 [SMILES: O=C1CN=C(c2ccccc2F)c2cc(Cl)ccc2N1CC(O)CO] |
| Pyrazolam | Agonist | 1 mg* | 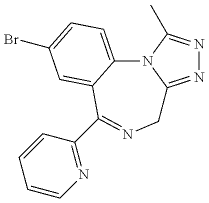 [SMILES: Cc1nnc2n1-c1ccc(Br)cc1C(c1ccccn1)=NC2] |
| Reclazepam | Agonist | N/A | 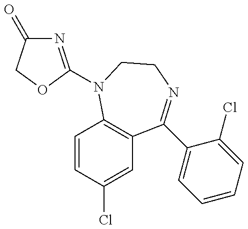 [SMILES: O=C1COC(N2CCN=C(c3ccccc3Cl)c3cc(Cl)ccc32)=N1] |
| Remimazolam | Agonist | N/A | 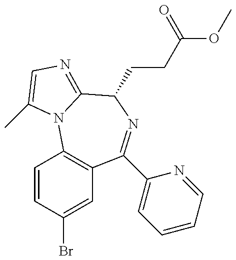 |
| Rilmazafone | Agonist (Prodrug) | N/A | 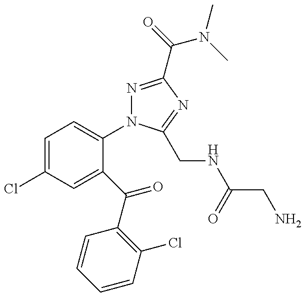 [SMILES: CN(C)C(=O)c1nc(CNC(=O)CN)n(-c2ccc(Cl)cc2C(=O)c2ccccc2Cl)n1] |
| Ripazepam | Agonist | N/A | 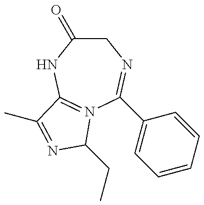 |
| Ro48-6791 | Agonist | N/A | 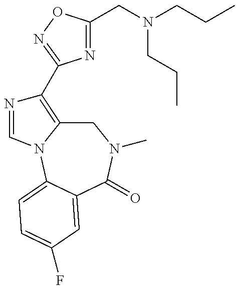 [SMILES: CCCN(CCC)Cc1nc(-c2ncn3c2CN(C)C(=O)c2cc(F)ccc2-3)no1] |
| SH-053-R-CH3-2′F | Agonist | N/A | 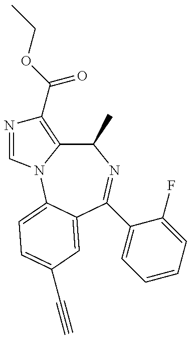 [SMILES: C#Cc1ccc2c(c1)C(c1ccccc1F)=N[C@H](C)c1c(C(=O)OCC)ncn1-2] |
| Sulazepam | Agonist | N/A | 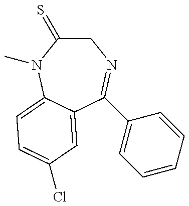 [SMILES: CN1C(=S)CN=C(c2ccccc2)c2cc(Cl)ccc21] |
| Temazepam (RESTORIL ™, NORMISON ™, EUHYPNOS ™, TEMAZE ™, TENOX ™) | Agonist | 20 mg* | 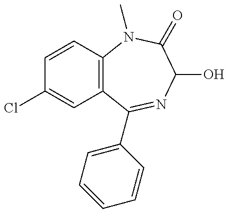 [SMILES: CN1C(=O)C(O)N=C(c2ccccc2)c2cc(Cl)ccc21] |
| Tetrazepam (CLINOXAN ™, EPSIPAM ™, MYOLASTAN ™, MUSARIL ™, RELAXAM ™, SPASMORELAX ™) | Agonist | 100 mg* Typical dose 50- 300 mg/day in divided doses | 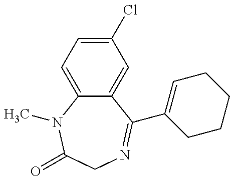 [SMILES: CN1C(=O)CN=C(C2=CCCCC2)c2cc(Cl)ccc21] |
| Tofisopam | Agonist | Typical dose 50- 300 mg/day in divided doses | 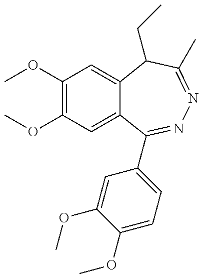 |
| Triazolam (APO-TRIAZO ™, HALCION ™, HYPAM ™, TRILAM ™) | Agonist | 0.25 mg* | 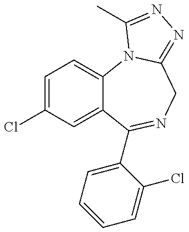 [SMILES: Cc1nnc2n1-c1ccc(Cl)cc1C(c1ccccc1Cl)=NC2] |
| Triflubazam | Agonist | N/A | 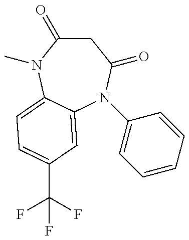 [SMILES: CN1C(=O)CC(=O)N(c2ccccc2)c2cc(C(F)(F)F)ccc21] |
| Uldazepam | Agonist | N/A | 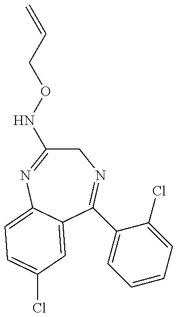 [SMILES: C=CCONC1=Nc2ccc(Cl)cc2C(c2ccccc2Cl)=NC1] |
| Zapizolam | Agonist | N/A | 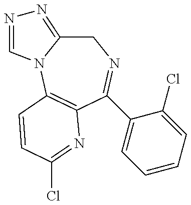 [SMILES: Clc1ccc2c(n1)C(c1ccccc1Cl)=NCc1nncn1-2] |
| Zolazepam | Agonist | N/A | 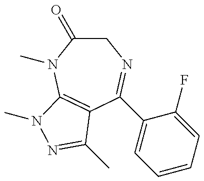 [SMILES: Cc1nn(C)c2c1C(c1ccccc1F)=NCC(=O)N2C] |
| Zomebazam | Agonist | N/A | 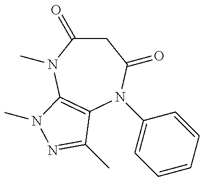 [SMILES: Cc1nn(C)c2c1N(c1ccccc1)C(=O)CC(=O)N2C] |

*Equivalent dose to 10 mg diazepam
N/A = not available

### Subunit Selective Positive Allosteric Modulators (SS-PAM) of the GABA-A Receptor

Also contemplated for use herein are subunit selective positive allosteric modulators of the GAB A-A receptor, particularly those that are selective for the α2 and/or α3 subunits. Agents that are selective for the α1, α4, α5, and/or α6 subunits are not contemplated for use with the methods and formulations described herein.

Also contemplated herein are positive allosteric modulators that act as agonists (e.g., full agonists or partial agonists) at the α2 and/or α3 subunits, while acting as an antagonist or inverse agonist at one or more of the α1, α4, α5, and/or α6 subunits.

Exemplary subunit selective positive allosteric modulators that have efficacy at the α2 and/or α3 subunits of the GABA-A receptor are listed in Table 2, below. In one embodiment, the subunit selective positive allosteric modulator used with the methods described herein is L838,417.

TABLE 2
Exemplary SS-PAMs

| | | Dose in humans | |
|---|---|---|---|
| | | for anti-anxiety, | |
| | Effect at | anti-convulsive, | |
| | GABA | or sedative | |
| Drug | receptor | effects | Structure |
| SL-651,498 | Full agonist α2,3 selective | N/A | 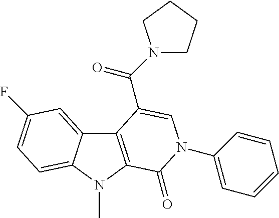 [SMILES: Cn1c2ccc(F)cc2c2c(C(=O)N3CCCC3)cn(-c3ccccc3)c(=O)c21] |
| Pagoclone | Partial agonist α2,3 selective | N/A | 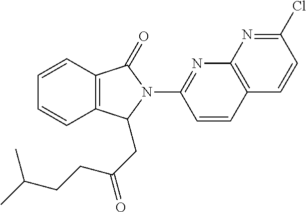 [SMILES: CC(C)CCC(=O)CC1c2ccccc2C(=O)N1c1ccc2ccc(Cl)nc2n1] |
| Alpidem (ANANXYL ™) | Agonist α3 subtype selective | N/A | 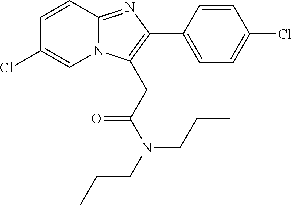 [SMILES: CCCN(CCC)C(=O)Cc1c(-c2ccc(Cl)cc2)nc2ccc(Cl)cn12] |
| Zaleplon (SONATA ™, STARNOC ™, ANDANTE ™) | Full agonist α1 selective Acts on α2,3 at higher doses Ultra short- acting | 20 mg* | 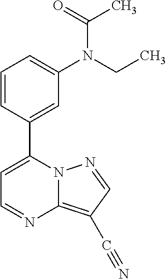 [SMILES: CCN(C(C)=O)c1cccc(-c2ccnc3c(C#N)cnn23)c1] |
| Adipiplon | Partial agonist α3 selective | N/A | 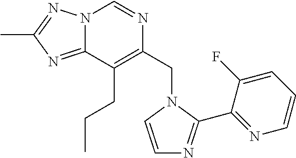 [SMILES: CCCc1c(Cn2ccnc2-c2ncccc2F)ncn2nc(C)nc12] |
| ELB-139 | Partial agonist Highest affinity for α3 subtype but highest efficacy at the α1 and α2 subunits | N/A | 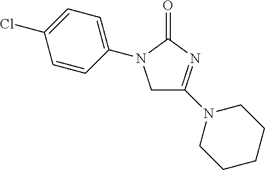 [SMILES: O=C1N=C(N2CCCCC2)CN1c1ccc(Cl)cc1] |
| L-838,417 | Partial agonist Selective for α2, α3 (and possibly α5) subunits | N/A | 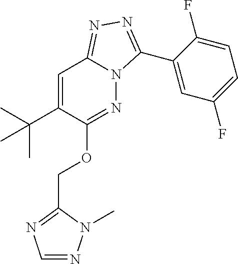 [SMILES: Cn1ncnc1COc1nn2c(-c3cc(F)ccc3F)nnc2cc1C(C)(C)C] |
| NS-2664 | Partial agonist α2,3 subtype selective | N/A | 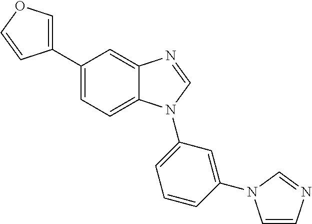 [SMILES: c1cc(-n2ccnc2)cc(-n2cnc3cc(-c4ccoc4)ccc32)c1] |
| NS-2710 | Partial agonist α2,3 subtype selective | N/A | 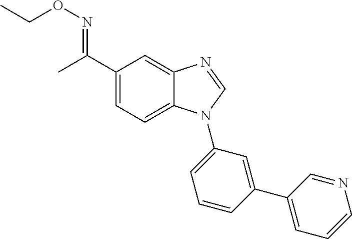 [SMILES: CCO/N=C(\C)c1ccc2c(c1)ncn2-c1cccc(-c2cccnc2)c1] |
| NS-11394 | Agonist α3,5 subtype selective | N/A | 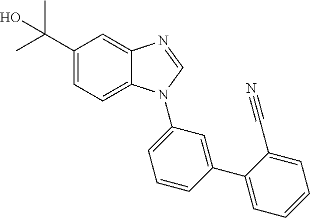 [SMILES: CC(C)(O)c1ccc2c(c1)ncn2-c1cccc(-c2ccccc2C#N)c1] |
| SB-205,384 | Agonist α3,5,6 subtype selective | N/A | 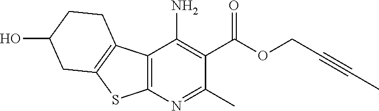 [SMILES: CC#CCOC(=O)c1c(C)nc2sc3c(c2c1N)CCC(O)C3] |
| TP-003 | Partial agonist α2,3,5 subtype selective binding and α3 selective efficacy | N/A | 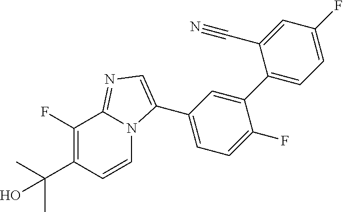 [SMILES: CC(C)(O)c1ccn2c(-c3ccc(F)c(-c4ccc(F)cc4C#N)c3)cnc2c1F] |
| TPA-023 | Partial agonist at α2,3 subunits Antagonist at α1,5 subunits | N/A | 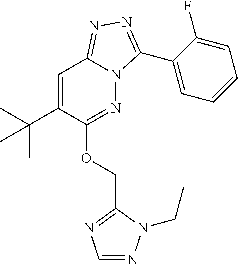 [SMILES: CCn1ncnc1COc1nn2c(-c3ccccc3F)nnc2cc1C(C)(C)C] |
| TP-13 | Partial agonist at α2,3 subunits | N/A | 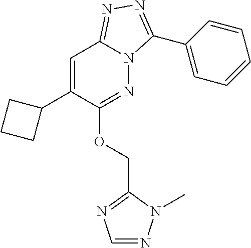 [SMILES: Cn1ncnc1COc1nn2c(-c3ccccc3)nnc2cc1C1CCC1] |
| U-89843A | Agonist at α1,3,6 subunits | N/A | 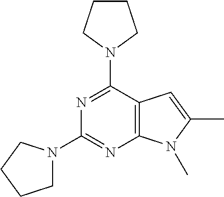 [SMILES: Cc1cc2c(N3CCCC3)nc(N3CCCC3)nc2n1C] |
| U-90042 | Agonist at α1,3,6 subunits | N/A | 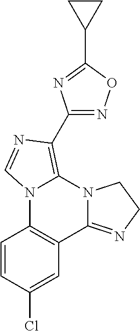 [SMILES: Clc1ccc2c(c1)C1=NCCN1c1c(-c3noc(C4CC4)n3)ncn1-2] |

### Neurosteroids

Also contemplated for use with the methods and formulations described herein, are neurosteroids that act as positive allosteric modulators of the GABA-A receptor.

As used herein the term “Neuroactive steroids (or neurosteroids)” refers to natural, synthetic, or semi-synthetic steroids that affect neuronal excitability through interaction with neurotransmitter-gated ion channels, including but not limited to, GABA-A, NMDA, and sigma receptors.

Neurosteroids can be classified into functional groups according to chemical structure and physiological activity and include estrogenic hormones, progestational hormones, and androgenic hormones. In one embodiment, the neurosteroids are progestational hormones, e.g., progestins or progestogens, and their derivatives and bioactive metabolites. Exemplary steroid hormones include those disclosed in Remington's Pharmaceutical Sciences, Gennaro et al., Mack Publishing Co. (18th ed. 1990), 990-993. As with all other classes of steroids, sterioisomerism is of fundamental importance with the sex hormones. As used herein, a variety of progestins (i.e., progesterone) and their derivatives, including both synthetic and natural products, can be used, as well as progestin metabolites such as progesterone.

The term “progesterone” as used herein refers to a member of the progestin family and includes a 21 carbon steroid hormone. Progesterone is also known as D4-pregnene-3,20-dione; A4-pregnene-3,20-dione; or pregn-4-ene-3,20-dione. As used herein a “synthetic progestin” is a molecule whose structure is related to that of progesterone, is synthetically derived, and retains the biological activity of progesterone.

Representative synthetic progestins include, but are not limited to, substitutions at the 17-position of the progesterone ring to introduce a hydroxyl, acetyl, hydroxyl acetyl, aliphatic, nitro, or heterocyclic group, modifications to produce 17α-OH esters (i.e., 17 α-hydroxyprogesterone caproate), as well as modifications that introduce 6-methyl, 6-ene, and 6-chloro substituents onto progesterone (i.e., medroxyprogesterone acetate, megestrol acetate, and chlomadinone acetate), and which retains the biological activity of progesterone. Such progestin derivatives include 5-dehydroprogesterone, 6-dehydro-retroprogesterone (dydrogesterone), allopregnanolone (allopregnan-3a, or 3β-o1-20-oηε), ethynodiol diacetate, hydroxyprogesterone caproate (pregn-4-ene-3,20-dione, 17-(1-oxohexyl)oxy); levonorgestrel, norethindrone, norethindrone acetate (19-norpregn-4-en-20-yn-3-one, 17-(acetyloxy)-,(17a)-); norethynodrel, norgestrel, pregnenolone, and megestrol acetate.

Useful progestins also can include allopregnone-3a or 3β, 20a or 20β-diol (see Merck Index 258-261); allopregnane-3,21-diol-11,20-dione; allopregnane-3 β, 17a-diol-20-one; 3,20-allopregnanedione, allopregnane, 3β,11β,17α,20β,21-ρεη\acute{ι}o1; allopregnane-3,17a,20,21-tetrol; allopregnane-3a or 3,1 i,17a,21-tetrol-20-one, allopregnane-3,17a or 20β-ιπo1; allopregnane-3β,17α,21-Mo1-11,20-dione; allopregnane-3,11,21-triol-20-one; allopregnane-3,17a,21-triol-20-one; allopregnane-3a or 3β-o1-20-oηε; pregnanediol; 3,20-pregnanedione; pregnan-3a-o1-20-one; 4-pregnene-20,21-diol-3,11-dione; 4-pregnene-1 i,17a,20,21-tetrol-3-one; 4-pregnene-17a,20,21-triol-3,11-dione; 4-pregnene-17a,20,21-triol-3-one, and pregnenolone methyl ether. Further progestin derivatives include esters with non-toxic organic acids such as acetic acid, benzoic acid, maleic acid, malic acid, caproic acid, and citric acid and inorganic salts such as hydrochloride, sulfate, nitrate, bicarbonate and carbonate salts. Other suitable progestins include alphaxalone, alphadolone, hydroxydione, and minaxolone.

In one embodiment, the neurosteroid is ganaxolone or gaboxadol (i.e., THIP).

TABLE 3
Exemplary Neurosteroids

| Drug | Structure |
|---|---|
| Acebrochol | 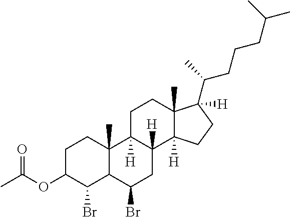 |
| Allopreg- nanolone | 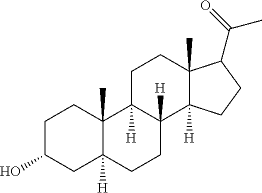 |
| Alpha- dolone | 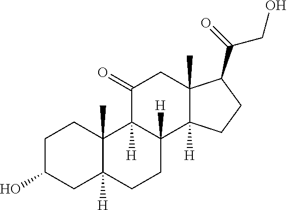 |
| Alpha- xolone | 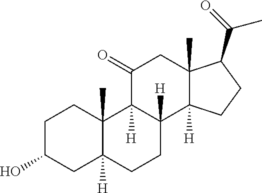 |
| Eltanolone | 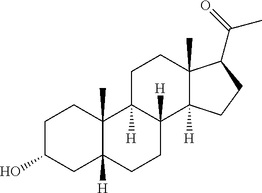 |
| Gaboxadol (THIP) | 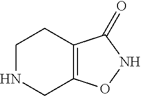 |
| Ganaxolone | 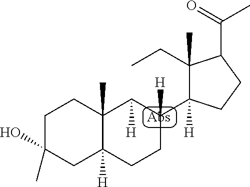 |
| Hydrox- ydione | 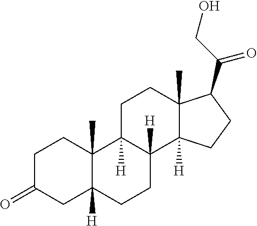 |
| Minaxolone | 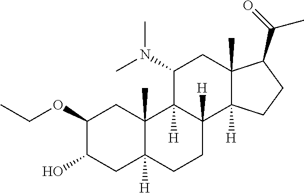 |
| Org 20599 | 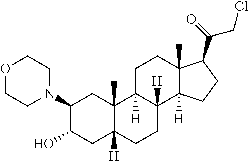 |
| Org 21465 | 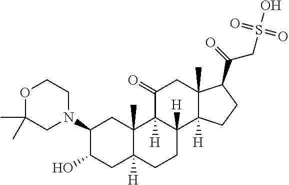 |
| THDOC (Tetrahydro- deoxy- corticoster- one) | 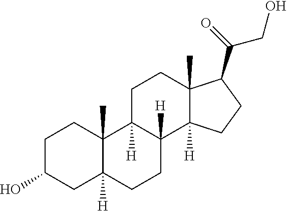 |

Other Non-Benzodiazepine Drugs that Increase the Response of GABA-A Receptors to GABA (“Non-BDZ GABA-A Enhancers”)

Also contemplated herein are the use of non-benzodiazepine positive allosteric modulators of GABA-A receptor. In one embodiment, non-BDZ GABA-A enhancers are contemplated for use in the treatment of ASDs or autism-like symptoms as described herein.

As used herein, the term “non-BDZ GABA-A enhancers” refers to a class of psychoactive drugs that have a similar therapeutic action to benzodiazepines and often bind to the benzodiazepine site of the GABA receptor, exhibit similar benefits, indications, and side effects despite being unrelated to benzodiazepines on the molecular or chemical level. Non-BDZ GABA-A enhancers act as positive allosteric modulators of the GAB A-A receptor, thereby facilitating the entry of chloride ions through the channel of the GABA-A receptor.

Non-BDZ GABA-A enhancers can be separated, in part, into different groups based on structure. These groups include the (i) imidizopyridines, (ii) pyrazolopyrimidines, (iii) cyclopyrrolones, (iv) β-carbolines, (v) neurosteroids, and (vi) others. Examples of each class of non-BDZ GABA-A enhancers are provided in Table 3 below or in the section on Neurosteroids.

In some embodiments, the non-BDZ GABA-A enhancer is a full agonist at the GABA-A receptor. In other embodiments, the non-BDZ GABA-A enhancer is a partial agonist at the GABA-A receptor. In some embodiments, the non-BDZ GABA-A enhancer is an anxiolytic. In other embodiments, the non-BDZ GABA-A enhancer is a short-acting anxiolytic. In other embodiments, the non-BDZ GABA-A enhancer is a long-acting anxiolytic.

In some embodiments, the non-BDZ GABA-A enhancer is a prodrug, although active metabolites of such prodrugs are also contemplated for use as described herein.

While the degree of usefulness of the agents listed in Table 4 may vary based on the characteristics of the agent, one can easily test if an agent is useful at low doses for the treatment of autism, e.g., as described herein in the Dosage and Administration section.

TABLE 4
List of exemplary non-BDZ GABA-A enhancers

| | | Dose in humans | |
|---|---|---|---|
| | | for anti-anxiety, | |
| | Effect at | anti-convulsive, | |
| | GABA | or sedative | |
| Drug | receptor | effects | Structure |
| Abecarnil | Partial agonist | N/A | 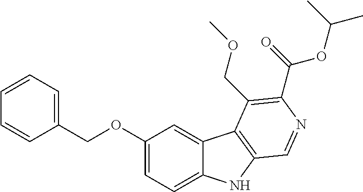 [SMILES: COCc1c(C(=O)OC(C)C)ncc2[nH]c3ccc(OCc4ccccc4)cc3c12] |
| Gedocarnil | Agonist | N/A | 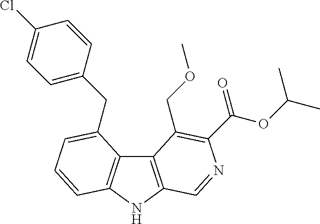 [SMILES: COCc1c(C(=O)OC(C)C)ncc2[nH]c3cccc(Cc4ccc(Cl)cc4)c3c12] |
| ZK-93423 | Agonist | N/A | 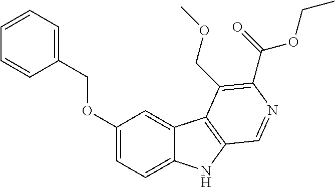 [SMILES: CCOC(=O)c1ncc2[nH]c3ccc(OCc4ccccc4)cc3c2c1COC] |
| Eszopiclone (LUNESTA ™) | Agonist | 3 mg* | 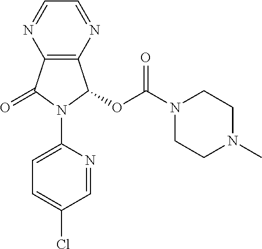 [SMILES: CN1CCN(C(=O)O[C@H]2c3nccnc3C(=O)N2c2ccc(Cl)cn2)CC1] |
| pazinaclone | Partial agonist | N/A | 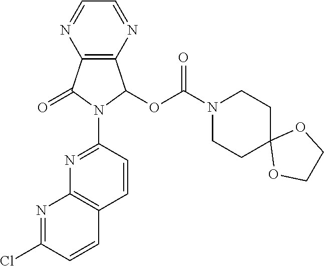 [SMILES: O=C(OC1c2nccnc2C(=O)N1c1ccc2ccc(Cl)nc2n1)N1CCC2(CC1)OCCO2] |
| Suproclone | Agonist | N/A | 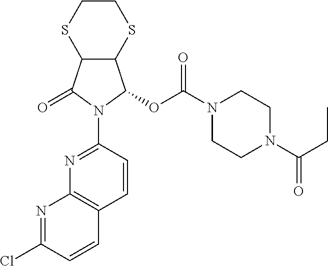 [SMILES: CCC(=O)N1CCN(C(=O)O[C@H]2C3SCCSC3C(=O)N2c2ccc3ccc(Cl)nc3n2)CC1] |
| Suriclone | Agonist | N/A | 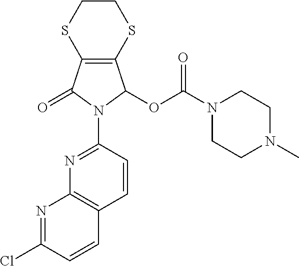 [SMILES: CN1CCN(C(=O)OC2C3=C(SCCS3)C(=O)N2c2ccc3ccc(Cl)nc3n2)CC1] |
| Zopiclone (IMOVANE ™) | Full agonist | 15 mg* | 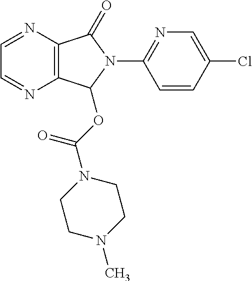 [SMILES: CN1CCN(C(=O)OC2c3nccnc3C(=O)N2c2ccc(Cl)cn2)CC1] |
| Necopidem | Agonist | N/A | 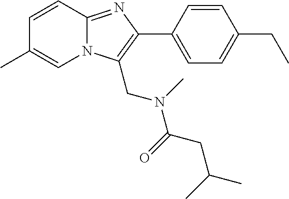 [SMILES: CCc1ccc(-c2nc3ccc(C)cn3c2CN(C)C(=O)CC(C)C)cc1] |
| Divaplon (RU-32698) | Partial agonist | N/A | 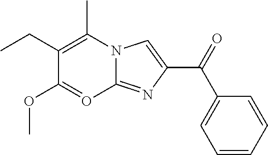 |
| Fasiplon | Agonist | N/A | 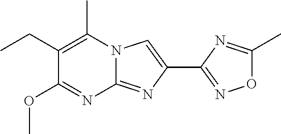 [SMILES: CCc1c(OC)nc2nc(-c3noc(C)n3)cn2c1C] |
| Lorediplon | Agonist | N/A | 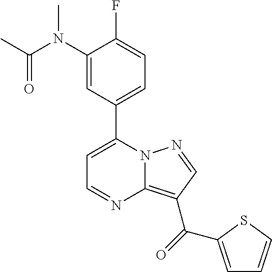 [SMILES: CC(=O)N(C)c1cc(-c2ccnc3c(C(=O)c4cccs4)cnn23)ccc1F] |
| Ocinaplon | Agonist | N/A | 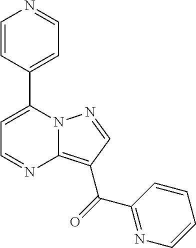 [SMILES: O=C(c1ccccn1)c1cnn2c(-c3ccncc3)ccnc12] |
| Panadiplon | Partial agonist | N/A | 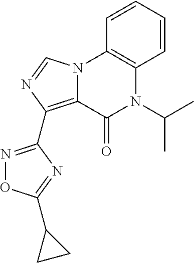 [SMILES: CC(C)n1c(=O)c2c(-c3noc(C4CC4)n3)ncn2c2ccccc21] |
| Taniplon | Agonist | N/A | 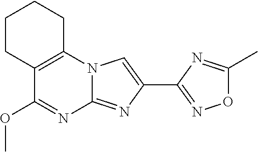 [SMILES: COc1nc2nc(-c3noc(C)n3)cn2c2c1CCCC2] |
| CGS-8216 | Agonist | N/A | 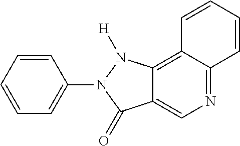 [SMILES: O=c1c2cnc3ccccc3c2[nH]n1-c1ccccc1] |
| CGS-9896 | Agonist | N/A | 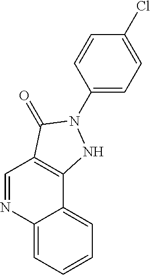 [SMILES: O=c1c2cnc3ccccc3c2[nH]n1-c1ccc(Cl)cc1] |
| CGS-13767 | Agonist | N/A | 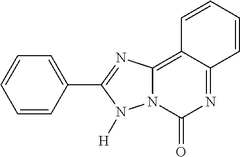 [SMILES: O=c1nc2ccccc2c2nc(-c3ccccc3)[nH]n12] |
| CGS-20625 | Agonist | N/A | 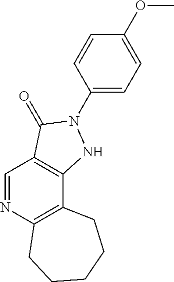 [SMILES: COc1ccc(-n2[nH]c3c4c(ncc3c2=O)CCCCC4)cc1] |
| GBLD-345 | Full agonist | N/A | 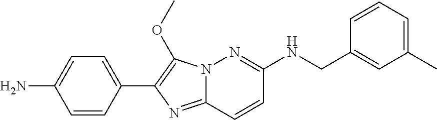 [SMILES: COc1c(-c2ccc(N)cc2)nc2ccc(NCc3cccc(C)c3)nn12] |
| Pipequaline | Partial agonist | N/A | 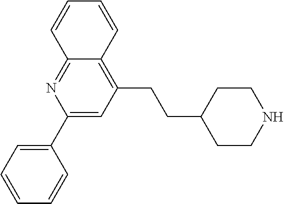 [SMILES: c1ccc(-c2cc(CCC3CCNCC3)c3ccccc3n2)cc1] |
| ROD-188 | Agonist | N/A | 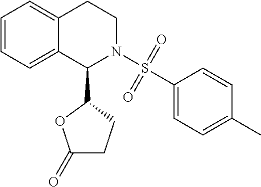 [SMILES: Cc1ccc(S(=O)(=O)N2CCc3ccccc3[C@@H]2[C@@H]2CCC(=O)O2)cc1] |
| RWJ-51204 | Partial agonist | N/A | 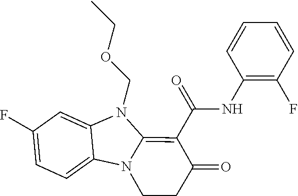 [SMILES: CCOCN1C2=C(C(=O)Nc3ccccc3F)C(=O)CCN2c2ccc(F)cc21] |
| Stiripentol (DIACOMIT ™) | | 50-100 mg/kg/day (max daily dose 4 g) | 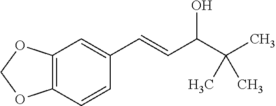 [SMILES: CC(C)(C)C(O)/C=C/c1ccc2c(c1)OCO2] |
| Y-23684 | Partial agonist Non- selective | N/A | 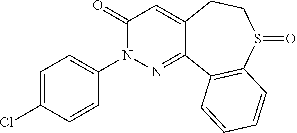 [SMILES: O=c1cc2c(nn1-c1ccc(Cl)cc1)-c1ccccc1S(=O)CC2] |

*Equivalent dose to 10 mg diazepam
N/A = not available

### Dosage and Administration

### Dosage—General

In one aspect, the methods described herein provide a method for treating autism, autism-like behavior, cognitive deficit and/or impaired context-dependent spatial memory (e.g., anti-social behaviors, Dravet syndrome, among others) in a subject. In one embodiment, the subject can be a mammal. In another embodiment, the mammal can be a human, although the approach is effective with respect to all mammals.

The method comprises administering to the subject an effective amount of a pharmaceutical composition comprising a low dose of an agent that increases GABAergic signaling, in a pharmaceutically acceptable carrier. What is critical with respect to the dosage and unit dose formulations prepared to deliver effective amounts of the agents described herein is that the dose be substantially lower than that which results in substantial sedation or anxiety-reducing or anti-convulsive effects in a given individual. Dosages well below, e.g., at least 50% or more below, the dosage effective for sedative, anti-convulsive effects or anxiolytic effects are surprisingly found to be effective for the treatment of autism or autism-like symptoms.

In one embodiment, the agent that increases GABAergic signaling is a benzodiazepine (e.g., clonazepam). In another embodiment, the agent that increases GABAergic signaling is a subunit selective positive allosteric modulator or a non-BDZ GABA-A enhancer, such as L838,417. The dosage range for the agent depends upon the potency, and includes amounts large enough to produce the desired effect, e.g., rescue of autism. The dosage should not be so large as to cause unacceptable adverse side effects. Generally, the dosage will vary with the type of inhibitor (e.g., small molecule, etc.), and with the age, condition, and sex of the patient. The dosage can be determined by one of skill in the art and can also be adjusted by the individual physician in the event of any complication.

In some embodiments and depending on the agent selected, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight.

### Determining Dosage Using a Calculation Based on Anti-Anxiety, Anti-Convulsive, Hypnotic/Sedative Effects

In one embodiment, the dose of a GABAergic signaling agent is calculated based on the dose of the agent that is typically used in a subject to elicit an anxiolytic, an anti-convulsive or an sedative effect. One of skill in the art can easily choose the endpoint (e.g., anxiolytic effect vs. sedative effect vs. anti-convulsant effect) for the drugs listed in Tables 1 or 2. For example, it is known that some of the agents in Tables 1 and 2 will act preferentially as anxiolytics and produce very little sedation in the subject and with such agents, one of skill in the art would use the dose required to produce an anxiolytic effect/endpoint to determine the starting dose from which to calculate an appropriate dose for the treatment of autism. The inventors have found that the agents in Tables 1 and 2 can be used for the treatment of autism at a dose of e.g., 10% of the dose used to achieve an endpoint of anti-anxiety, sedative or anti-convulsive effects. Since the pharmacokinetics (e.g., the half-life of the drug, drug metabolism, etc.) are dependent on the agent itself, and not the dose of the agent administered, one of skill in the art can assume that a lower dose will be metabolized in the same manner as the higher dose. That is, one can simply calculate the dose useful for treating autism in a subject by calculating a percentage of the dose used to achieve anxiolytic, sedative, or anti-convulsive effects in the same subject or an equivalent subject (e.g., humans).

In some embodiments, the dose of an agent used with the methods and formulations described herein in Table 1 or 2 is less than 1% of the dose of the same agent that is used to achieve an anxiolytic, sedative or anti-convulsive effect in the subject. In other embodiments, the dose of an agent in Table 1 or 2 is less than 2%, less than 5%, less than 10%, less than 15%, less than 20%, less than 25%, less than 30%, less than 40%, or less than 50% of the dose of the same agent that is used to achieve an anxiolytic, sedative or anti-convulsive effect in the subject.

### Determining Dosage Based on Equivalence to Diazepam

The following is a sample calculation to determine the dosage of clonazepam for the treatment of autism based on the equivalent therapeutic dose of clonazepam to achieve the therapeutic effects of 10 mg diazepam (e.g., anti-anxiety, anti-convulsive effects). This sample calculation can be applied to any drug where an equivalent dosage to the effects of diazepam is known, and is not limited to the use of clonazepam. Clonazepam is used herein as illustration of an exemplary positive allosteric modulator of the GABA-A receptor. Likewise, the following sample calculation demonstrates how to calculate a dose for the treatment of an autism spectrum disorder that is 1/10 or 10% of the therapeutic dose used for anxiety or convulsions. The 10% value is intended only to be illustrative and is not limiting.

By convention, the dose of some positive allosteric modulators of the GABA-A receptor are expressed as a dose “equivalent” to the classical benzodiazepine diazepam (VALIUM™). For example, a dose of 0.5 mg of clonazepam is considered to have an equivalent efficacy to a dose of 10 mg of diazepam. Since the daily dose for treatment of anxiety and convulsions is known for diazepam, one of skill in the art can calculate the dose needed for each agent (e.g., in Table 1 and/or 2) to achieve a similar therapeutic effect to diazepam. Diazepam is typically administered at a dose between 2-10 mg, 2-4 times daily (e.g., 4 mg to 40 mg daily dose) to achieve an anxiolytic or anti-convulsive effect. Thus, taking clonazepam as an example (equivalent dose of 0.5 mg to 10 mg diazepam), the equivalent dose would be 0.1 mg-0.5 mg, 2-4 times daily or a daily dose of 0.2 mg-2 mg.

The inventors' work as described in the Examples section indicates that a dose of e.g., 10% of the dose necessary to achieve anti-anxiety, anti-convulsive or sedative effects is useful for the treatment of autism. Thus, one of skill in the art can then calculate the daily dose of e.g., clonazepam necessary for the treatment of autism by simply dividing the dose necessary for anti-anxiety, anti-convulsive, or sedative effects by that number (e.g., 10). Therefore, in one embodiment, the daily dose of clonazepam contemplated for treatment of autism is 0.02 mg-0.2 mg (1/10 of the daily dose calculated above).

One of skill in the art will recognize that the current formulations for treatment of e.g., anxiety will be too high for the treatment of autism (e.g., clonazepam dosage formulations in the U.S. include a 0.5 mg, a 1 mg, and a 2 mg tablet). Thus also contemplated herein are low-dose formulations of clonazepam for the treatment of autism spectrum disorders. One of skill in the art of pharmacology or medicine can determine the appropriate dosing schedule based on the pharmacokinetics of the agent to achieve a daily dose of e.g., 0.02 mg-0.2 mg and can determine the best low-dose formulation to use (e.g., an appropriate unit dose for a formulation). For example, the half-life of clonazepam is 30-40 hours, making it suitable for once daily dosing. A dosage formulation of 0.02 mg or 0.1 mg tablets are specifically contemplated herein for clonazepam. As used herein, the term “unit dose” when referring to a formulation refers to a dose at e.g., 1/10 that which is used to achieve a sedative, anti-anxiety, or anti-convulsive effect.

### Determining Dosage Based on a Dose-Response Curve

In some cases, the equivalent dose of an agent to diazepam or the dosage necessary to achieve sedation, anti-anxiety or anti-convulsive effects may not be known. In such cases it is easy to determine a starting dose for that agent using a dose-response study in a subject or population of subjects. One of skill in the art of pharmacology or medicine can make an educated guess at the range of doses necessary to elicit an effect in human subjects based on prior animal studies.

Typically, the drug will be administered to a group of subjects at different concentrations and the endpoint effect monitored (e.g., anti-anxiety, anti-convulsive or sedative effects). In general, most drugs follow a logarithmic dose response curve, where at small doses very little effect is observed and a maximal dose is evident, where an increase in dose of the agent no longer produces a measurable increase in the therapeutic effect. From the maximal dose, one of skill in the art can also calculate the dose that produces 50% of the effect (e.g., the ED50 (effective dose)) and other pharmacological parameters.

In one embodiment, the dose-response study can be performed in a subset of “normal” subjects, wherein the subjects are substantially free of psychiatric, seizure or sleep disorders. In such cases, the therapeutic endpoint to be measured is generally sedation, although anti-anxiety can also be measured in some cases. From the dose-response curve one of skill in the art can logically begin treatment of a subject with autism using a dose that is e.g., 10% of the dose to achieve sedation, anti-anxiety, or anti-convulsive effect in the dose-response study. As an example, the “dose to achieve sedation” (or other classical effect of GAB A-A receptor enhancers) can refer to either the maximal dose, the minimum effective dose (e.g., the threshold dose) or the ED50. For clarity, when there is any question, dosage refers to the ED50 to achieve one or more classical effects of benzodiazepine or non-BDZ GABA-A receptor enhancers, as described herein. Thus, a “low dose” as the term is used herein is calculated in reference to the ED50 for sedative, anxiolytic or anti-convulsive effect of such drug. In one embodiment, the “dose to achieve sedation” (or other classical effect of a GABA-A receptor enhancer) refers to an effective dose in which at least 50% of subjects in a population (e.g., normal or wildtype subjects) will exhibit a sedative, anxiolytic or anti-convulsive effect (e.g., ED50). Typically, one of skill in the art will appreciate that it is appropriate to begin treatment with a lower dose of the agent to minimize side effects, and slowly increase the dose while monitoring the patient for an appropriate therapeutic effect.

In another embodiment, the dose-response study can be performed in a subset of subjects having an autism spectrum disorder and the therapeutic endpoint measured is an improvement in at least one of the following e.g., social interactions, repetitive behaviors, cognitive deficit, or language development. One will appreciate that the most useful therapeutic endpoints for a dose-response study are endpoints that demonstrate an acute change upon administration of the drug, thus the endpoints most easily measured for autistic spectrum disorders include number of social interactions or the frequency of repetitive behaviors. This type of dose-response study is directly applicable for the treatment of subjects having autism and does not require any further calculations of dosage. One can easily determine a minimum effective dose, the ED50 and the maximal dose for the treatment of autism using such a study.

### Exemplary Doses of Clonazepam in Humans

In one embodiment, the daily dose of clonazepam is in the range of 0.0125 mg to 0.3 mg. In other embodiments, the daily dose of clonazepam is between 0.005 mg to 0.01 mg, between 0.01 mg to 0.02 mg, between 0.0125 mg and 0.02 mg, between 0.015 to 0.02 mg, between 0.0175 to 0.02 mg, between 0.02 mg to 0.04 mg, between 0.025 mg to 0.04 mg, between 0.03 to 0.04 mg, between 0.02 mg to 0.05 mg, between 0.03 mg to 0.0625 mg, between 0.035 mg to 0.0625 mg, between 0.04 mg to 0.3 mg, between 0.045 mg to 0.3 mg/kg, between 0.05 mg to 0.3 mg, between 0.055 mg to 0.3 mg, between 0.06 mg to 0.3 mg, between 0.07 to 0.3 mg, between 0.08 to 0.3 mg, between 0.09 to 0.3 mg, between 0.1 to 0.3 mg, between 0.15 to 0.3 mg, between 0.2 to 0.3 mg, or between 0.25 to 0.3 mg.

In other embodiments, the dose of clonazepam is less than 40% of the dose of clonazepam that induces sedation, anti-convulsive activity or anxiolytic activity; in other embodiments, the dose of clonazepam is less than 30% lower, less than 20%, less than 10%, less than 5%, less than 2%, less than 1% or less of the dose of clonazepam that produces sedation, anti-convulsive activity or anxiolytic activity.

### Administration

Administration of the doses recited above can be repeated for a limited period of time or indefinitely as therapeutic efficacy and the need to continue treatment dictates. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated. A therapeutically effective amount is an amount of an agent that is sufficient to produce a statistically significant, measurable change in a therapeutic endpoint such as a reduction in one or more symptoms of autism, including but not limited to, improvement in social interactions and/or a reduction in repetitive behaviors (see “Efficacy Measurement” below). Such effective amounts can be gauged in clinical trials as well as animal studies for a given agent. Agents useful in the methods and compositions described herein can be administered topically, intravenously (e.g., continuous infusion), orally, by inhalation, intraperitoneally, intramuscularly, subcutaneously, intracavity, and can be delivered by peristaltic means, if desired, or by other means known by those skilled in the art. In general, it is preferred that the agent is administered orally or is prepared as a formulation to be administered orally. The agent can be administered systemically, if so desired.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle. The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous infusion (e.g., an osmotic mini-pump) sufficient to maintain concentrations in the blood in the ranges specified for in vivo therapies are contemplated.

### Pharmaceutical Compositions and Formulations

The present invention includes, but is not limited to, therapeutic compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. The compositions can be prepared as injectable either as liquid solutions or suspensions, however, solid forms suitable for solution, or suspensions, in liquid prior to use are specifically contemplated herein. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the drug) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

When preparing a formulation for administration, it is important that the blood brain barrier permeability to the agent remains intact; that is, that the modified drug or formulation can still pass through the blood brain barrier to act on GABA-A receptors in the central nervous system.

### Oral Formulations

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, matrix-forming compositions and coating compositions.

“Carrier” also includes all components of the coating composition which may include plasticizers, pigments, colorants, stabilizing agents, and glidants. delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et. al., (Media, Pa.: Williams and Wilkins, 1995) which provides information on carriers, materials, equipment and processes for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides. Additionally, the coating material may contain conventional carriers such as plasticizers, pigments, colorants, glidants, stabilization agents, pore formers and surfactants.

Optional pharmaceutically acceptable excipients present in the drug-containing or comprising tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pre-gelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pre-gelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Some of the materials which are suitable as binders can also be used as matrix-forming materials such as hydroxypropyl methyl cellulose, ethyl cellulose, and microcrystalline cellulose.

Lubricants are used to facilitate tablet manufacture. Examples of suitable lubricants include, but are not limited to, magnesium stearate, calcium stearate, stearic acid, glycerol behenate, polyethylene glycol, talc, and mineral oil. Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pre-gelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing or comprising carboxylate, sulfonate and sulfate ions. Examples of anionic surfactants include sodium, potassium, ammonium salts of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine. If desired, the tablets, beads, granules or particles may also contain minor amount of nontoxic auxiliary substances such as wetting or emulsifying agents, dyes, pH buffering agents, and preservatives.

A delayed-release portion is designed to prevent drug release until after a defined period of time or until a specific location in the body is reached. In the case of an orally delivered formulation, this would be in the upper part of the gastrointestinal (GI) tract or upon entry to the small intestines. Delayed release in an oral formulation can be achieved using enteric coatings. The enteric coated formulation remains intact or substantially intact in the stomach but dissolves and releases the contents of the dosage form once it reaches the small intestine. Other types of coatings can be used to provide delayed release following injection subcutaneously, intra-tissue or intramuscularly at a site near or at the area to be treated.

Extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. It should be understood that extended release dosage forms may include a total amount of drug that reaches or exceeds that which normally induces sedative, anxiolytic or anti-convulsive effects, but because it is formulated for extended release, the bioavailable portion of the drug at any given time results in an effective level below that which provides, e.g., anxiolytic, sedative or anti-convulsive effects.

Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof. In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is included of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer includes a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form including single or multiple units. Examples of multiple units include, but are not limited to, multilayer tablets and capsules containing or comprising tablets, beads, or granules.

An immediate release portion can be added to the extended release system by means of either applying an immediate release layer on top of the extended release core using a coating or compression process or in a multiple unit system such as a capsule containing or comprising extended and immediate release beads.

Extended release tablets containing or comprising hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing or comprising wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing or comprising composition with a selected coating material. The drug-containing or comprising composition may be, e.g., a tablet for incorporation into a capsule, a tablet for use as an inner core in a “coated core” dosage form, or a plurality of drug-containing or comprising beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Efficacy Measurement

The efficacy of a given treatment for an Autism Spectrum Disorder (ASD) can be determined by the skilled clinician. However, a treatment is considered “effective treatment,” as the term is used herein, if any one or all of the signs or symptoms of the disease is/are altered in a beneficial manner, other clinically accepted symptoms or markers of disease are improved, or even ameliorated, e.g., by at least 10% following treatment with an agent that comprises an agent that increases GABAergic signaling. At a minimum, treatment is considered effective if at least one or more of the following indicia of an ASD are reduced or ameliorated: (i) poor social interactions, (ii) incidence or number of repetitive behavior(s), (iii) cognitive deficit/learning impairment or (iv) impaired language development. In one embodiment, treatment is considered effective if there is an improvement in social interactions (e.g., eye contact etc.) and/or a reduction in the number or incidence of repetitive behaviors in an individual being treated as described herein. As used herein, the term “ameliorated” refers to at least a 10% improvement in one or more indicia of autism (e.g., at least at 20%, 30%, 40%, 50%, 75% or more improvement) or measured by an improvement of at least 1 point (e.g., at least 2, 3, 4, 5, 6, 7, 8, 9, 10, 15 points or more) on a standardized clinical scale (e.g., the CARS scale).

Efficacy can also be measured by failure of an individual to worsen as assessed by stabilization of the disease, hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of autistic behaviors such as impaired social interactions or repetitive behaviors; or (2) relieving the disease, e.g., causing regression of symptoms; and (3) preventing or reducing the likelihood of the development of autism, or preventing secondary diseases/disorders associated with the disease (e.g., seizures etc). An effective amount for the treatment of a disease means that amount which, when administered to a mammal in need thereof, is sufficient to result in effective treatment as that term is defined herein, for that disease. Efficacy of an agent can be determined by assessing indicators of the autism or autistic-like disease, such as e.g., anti-social behaviors, impaired social interactions, seizures, cognitive deficits, autism-spectrum behaviors, hyperactivity, attention deficit, delayed psychomotor development, sleep disorders, anxiety-like behaviors, restricted interests, etc.

One of skill in the art can diagnose and assess efficacy of a treatment for autism and autistic-like behavior using a grading scale such as the Childhood Autism Rating Scale (CARS). CARS is a diagnostic assessment method that rates children on a scale from one to four for various criteria, ranging from normal to severe, and yields a composite score ranging from non-autistic to mildly autistic, moderately autistic, or severely autistic. The scale is used to observe and subjectively rate the following fifteen items: relationship to people, imitation, emotional response, body, object use, adaptation to change, visual response, listening response, taste-smell-touch response and use, fear and nervousness, verbal communication, non-verbal communication, activity level, level and consistency of intellectual response, general impressions. Each of the fifteen criteria is rated by a parent, teacher or health professional and given a score of: (i) 1 normal for child's age, (ii) 2 mildly abnormal, (iii) 3 moderately abnormal, (iv) 4 severely abnormal. Midpoint scores of 1.5, 2.5, and 3.5 can also be used. Total CARS scores range from a fifteen to 60, with a minimum score of thirty serving as the cutoff for a diagnosis of autism on the mild end of the autism spectrum. Although the CARS study is mostly used in the diagnosis of children with an autism spectrum disorder, it can also be employed in adults to discern the presence and/or degree of autism in an adolescent or adult. In some embodiments, a cut-off of 27, rather than 30, is used when applying the CARS scale to adolescents or adults.

One of skill in the art can monitor the efficacy of a benzodiazepine or non-benzodiazepine anxiolytic for treatment of autism by regularly assessing a subject using the CARS grading scale. An agent is efficacious when the degree of severity of autism or autistic-like behavior is reduced, e.g., when the CARS score is lower e.g., by one point or more, preferably by 2, 3, 4, 5, 6, 7, 8, 9, 10, 11, 12, 13, 14, 15 points or more. Other tests known in the art for diagnosis of autism can be used with the methods described herein and include, but are not limited to, the Autism Diagnostic Interview-Revised (ADI-R), and/or the Autism Diagnostic Observation Schedule (ADOS).

### EXAMPLES

### Summary

The present disclosure describes methods effective in the rescue or amelioration of autism-like behavior, cognitive deficit, and context-dependent spatial memory. These methods are demonstrated to be effective in rescuing autism-like behavior, cognitive deficit, and context-dependent spatial learning and memory in well-established mouse genetic models of human Dravet syndrome and idiopathic autism.

In particular embodiments, these methods comprise the administration of low doses of a benzodiazepine to a human or other mammalian subject in order to rescue autism-like behavior, cognitive deficit, and context-dependent spatial memory. The efficacy of such low-dose treatment is demonstrated herein for mice at doses from 0.0125 mg/kg to 0.0625 mg/kg of clonazepam (CLZ). At such low doses, the administration of benzodiazepines, such as CLZ, to mammalian subjects will result in improvement of autism-like behavior, cognitive deficit, and context-dependent spatial learning and memory with much lower levels of sedation compared to mammalian subjects who receive typical higher doses of CLZ or other benzodiazepines that are used for treatment of anxiety, insomnia, or seizure.

The present disclosure is also directed to methods of rescuing or ameliorating indicia of Autism Spectrum disorders, autism-like behavior, cognitive deficit, and context-dependent spatial learning and memory in Dravet syndrome or idiopathic autism with α-2,3-specific GABA-A receptor positive allosteric modifiers. Such α-2,3-subunit selective GABA-A receptor positive allosteric modifiers include L838,417. In certain embodiments of such methods, the α-2,3-specific GABA-A receptor positive allosteric modifiers are administered to humans or other mammalian subjects at low doses in order to rescue autism-like behavior, cognitive deficit, and/or context-dependent spatial learning and memory without sedation or other side effects that may occur at higher doses. The efficacy of such treatment is demonstrated herein for treatment of mice with the α-2,3-specific GABA-A receptor positive allosteric modifier L838,417 at doses from 0.0125 mg/kg to 0.05 mg/kg.

The low doses of one or more benzodiazepine, including CLZ, and α-2,3-specific GABA-A receptor positive allosteric modifiers, including L838,417, are administered to a human or mammalian subject at a dosage that rescues autism-like behavior, cognitive deficit, and context-dependent spatial learning and memory in Dravet syndrome, idiopathic autism, or other related diseases. The dosage of such therapeutics will vary depending upon the specific drug used, the mammalian species to be treated, the nature of the disease, the frequency of administration, the manner of administration, the clearance of the agent from the host, and the like, but will be well below that administered for anxiety, sedation, or anti-convulsive therapy.

The effective amount of a therapeutic composition to be given to a particular mammalian subject will depend on a variety of factors, several of which will be different from subject to subject. Utilizing ordinary skill, a competent physician will be able to optimize dosage of the therapeutics described in the present disclosure.

### Example 1

### Autistic-Like Behavior in Scn1a^{+/-} Mice and Rescue by Enhanced GABA-Mediated Neurotransmission

Haploinsufficiency of the SCN1A gene encoding voltage-gated sodium channel Naᵥ1.1 causes Dravet syndrome, a childhood neuropsychiatric disorder including recurrent intractable seizures, cognitive deficit and autism-spectrum behaviors. The neural mechanisms responsible for cognitive deficit and autism-spectrum behaviors in Dravet syndrome are poorly understood. The inventors previously reported that mice with Scn1a haploinsufficiency exhibit hyperactivity, stereotyped behaviors, social interaction deficits and impaired context-dependent spatial memory.⁶¹ Olfactory sensitivity is retained, but novel food odors and social odors are aversive to Scn1a+/− mice. GABAergic neurotransmission is specifically impaired by this mutation, and selective deletion of Naᵥ1.1 channels in forebrain interneurons is sufficient to cause these behavioral and cognitive impairments. Remarkably, treatment with low-dose CLZ, a positive allosteric modulator of GABA-A receptors, completely rescued the abnormal social behaviors and deficits in fear memory in the mouse model of Dravet syndrome, demonstrating that they are caused by impaired GABAergic neurotransmission and not by neuronal damage from recurrent seizures. These results demonstrate a critical role for Naᵥ1.1 channels in neuropsychiatric functions and provide a potential therapeutic strategy for cognitive deficit and autism-spectrum behaviors in Dravet syndrome.

Dravet syndrome (DS), also called severe myoclonic epilepsy of infancy, is an intractable developmental epilepsy syndrome with seizure onset in the first year of life′. However, unlike other generalized epilepsy disorders, it is accompanied by characteristic neuropsychiatric comorbidities, including hyperactivity, attention deficit, delayed psychomotor development, sleep disorder, anxiety-like behaviors, impaired social interactions, restricted interests and severe cognitive deficits¹⁻⁶. These co-morbidities in DS overlap with symptoms of autism-spectrum disorders (ASD), and a recent study indicates that DS patients have autism-spectrum behaviors³. DS is caused by heterozygous loss-of-function mutations in the SCN1A gene⁷, which encodes the pore-forming α-subunit of the brain voltage-gated sodium channel type-1 (NaV1.1)⁸. As in DS, mice with heterozygous loss-of-function mutation in Scn1a (Scn1a+/−) have thermally induced and spontaneous seizures, premature death, ataxia and sleep disorder⁹⁻¹³. NaV1.1 channels are expressed in cell bodies and axon initial segments of excitatory and inhibitory neurons in the brain¹⁴⁻¹⁶ but deletion of NaV1.1 impairs sodium currents and action potential firing of GAB Aergic interneurons specifically because NaV1.1 is the primary sodium channel in those cells^{9,16}. Specific deletion of NaV1.1 channels in forebrain interneurons using a Cre-LoxP strategy recapitulates the symptoms of DS in mice¹⁷, confirming that loss of NaV1.1 in GABAergic interneurons causes this disease. Emerging genetic evidence implicates the SCN1A gene in autism¹⁸⁻²², and there is increasing evidence that dysfunction of GABAergic signaling is associated with ASDs²³⁻²⁵, leading to the proposal that elevation of the excitation/inhibition ratio in neocortical neurons is the primary cause of ASD²⁶⁻²⁹. The inventors investigated autism-related behaviors in Scn1a+/− mice and showed that they are caused by impaired GABAergic neurotransmission that can be rescued by drug treatment designed to enhance GABAergic inhibitory transmission.

Hyperactivity, Anxiety and Stereotypes in Scn1a+/− Mice

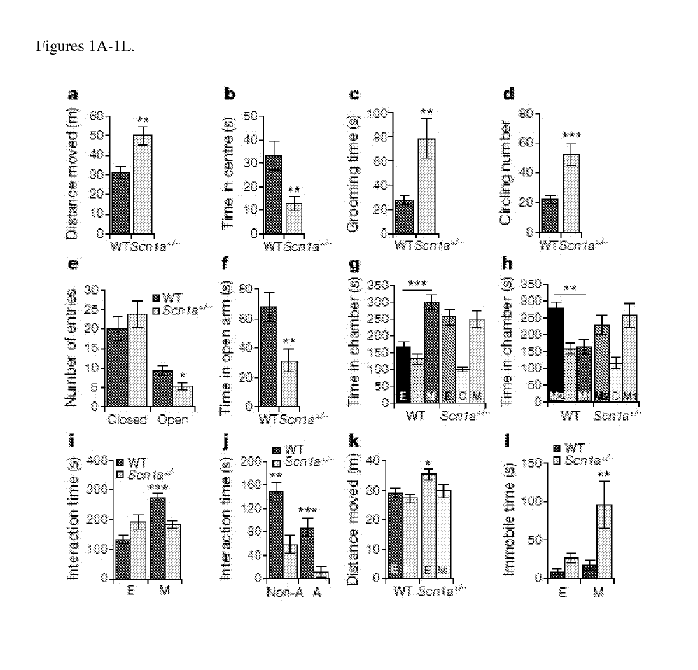

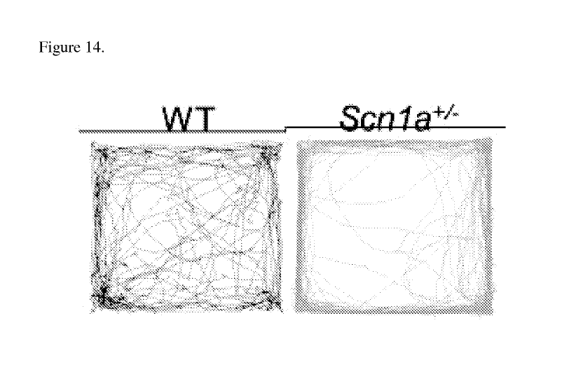

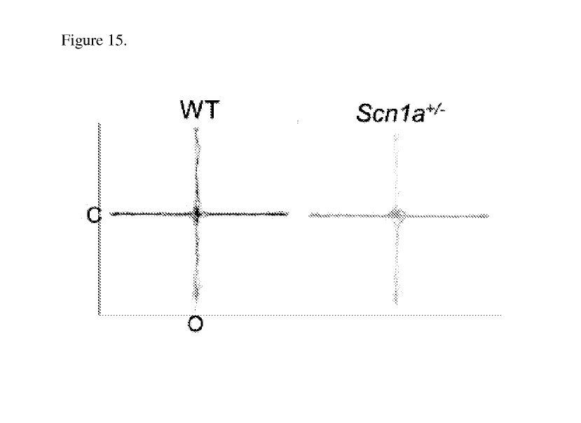

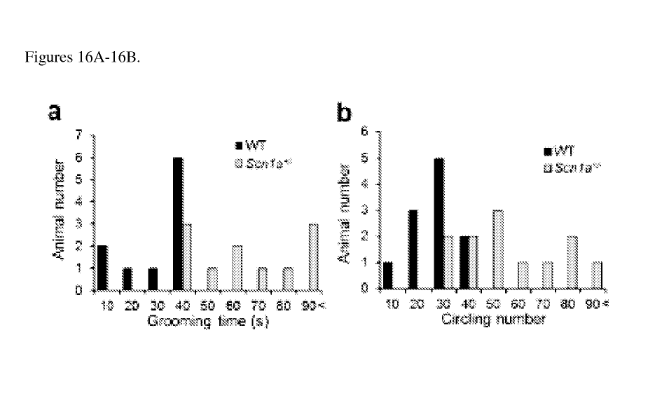

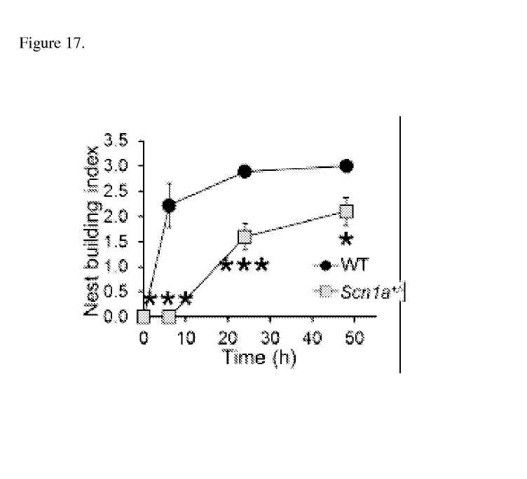

Homozygous Scn1a−/− mice developed severe ataxia and died on postnatal day (P) 15, whereas Scn1a+/− mice had spontaneous seizures and sporadic deaths beginning after P21 (ref. 9). Scn1a+/− mice develop multiple behavioral phenotypes, which are phenocopies of comorbidities in DS. During a 10-min open-field test, adult Scn1a+/− mice traveled significantly farther than wild-type (FIG. 1A), but spent less time in the centre of the open field (FIG. 1B and FIG. 14). Scn1a+/− mice also spent more time self-grooming than wild-type (FIG. 1c and FIG. 16A) and showed increased circling behavior (FIG. 1D and FIG. 16B). In the elevated plus maze, Scn1a+/− mice entered open arms less frequently compared with wild type (FIG. 1E), and spent less time in the open arms (FIG. 1F and FIG. 15). These observations indicate that Scn1a+/− mice exhibit hyperactivity, increased anxiety and increased stereotyped behaviors, which are phenocopies of autistic traits in DS. Scn1a+/− mice also have decreased nest-building ability compared to wild type (FIG. 17), which could indicate deficits in social behavior³⁰.

Scn1a+/− Mice have Deficits in Social Interaction

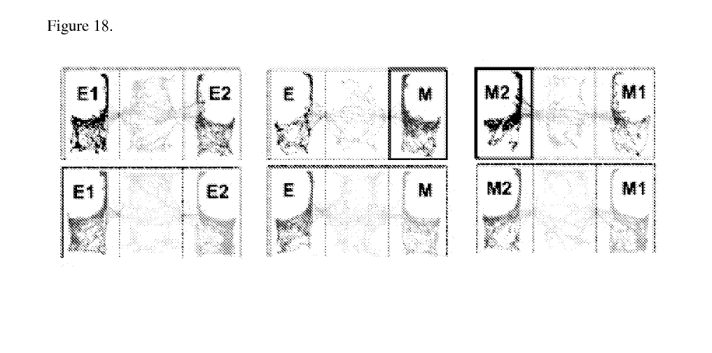

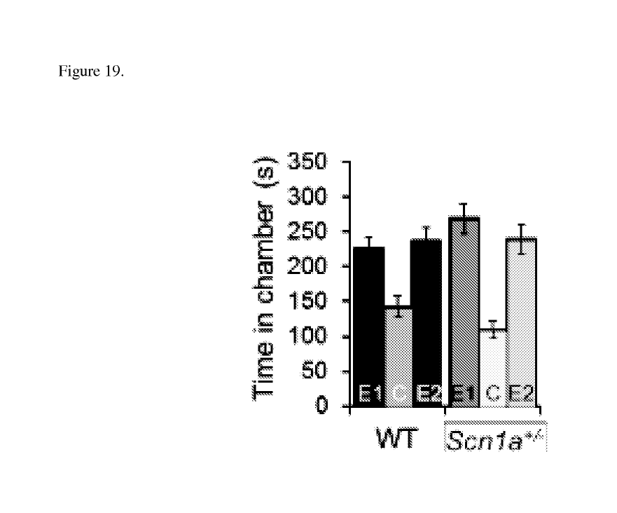

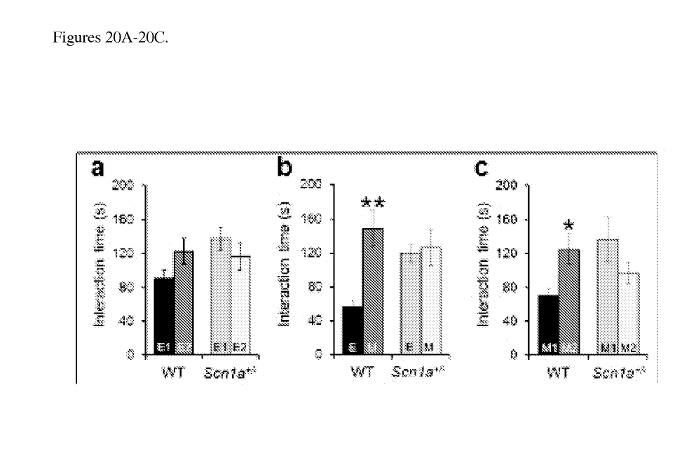

The inventors performed behavioral tests to assess deficits in social interaction, a prominent symptom of ASD³¹. A three-chamber experiment showed that Scn1a+/− mice have profound deficits in social interaction. Both Scn1a+/− and wild type had no preference for two empty cages, located in the right and the left chambers during a habituation period (FIGS. 18, 19, 20A). However, when a stranger mouse was put in the cage in one chamber, wild-type mice spent more time in the mouse-containing chamber than in the empty cage-containing chamber (FIG. 1G and FIG. 18), and interacted more extensively with peer mice than with the empty cage (FIG. 20B). In contrast, Scn1a+/− mice showed no preference for the stranger mouse (FIGS. 1G, 18 and 20B). When a second stranger mouse was placed in the unoccupied side chamber to assess the discrimination between a new and a familiar mouse, wild-type mice showed strong preference for the new mouse, but Scn1a+/− mice did not (FIGS. 1H, 18 and 20C), even though they have preference for new objects (see below). The inventors observed similar social deficits of Scn1a+/− mice in the open-field social interaction test. Scn1a+/− mice interacted significantly less with a caged stranger mouse in an open field compared with wild-type mice (FIGS. 1I and 21A). When both the inanimate object and the caged stranger mouse were introduced simultaneously, wild-type mice interacted significantly more with a caged stranger mouse than with an empty cage, whereas Scn1a+/− mice showed no preference for the caged mouse (FIGS. 22A, 22B). The inventors also examined reciprocal social interactions of freely moving Scn1a+/− and wild-type littermates with test mice. Scn1a+/− mice showed decreased duration of both non-aggressive and aggressive interactions (FIG. 1J). The inventors observed that Scn1a+/− mice exhibited increased immobilization behavior when they encountered the caged stranger mouse (FIG. 21B). Compared to wild-type, this immobilization decreased distance traveled (FIG. 1K) and increased immobilization time by 400% (FIG. 1L). Taken together, these results indicate that Scn1a+/− mice have profound deficits in social behavior.

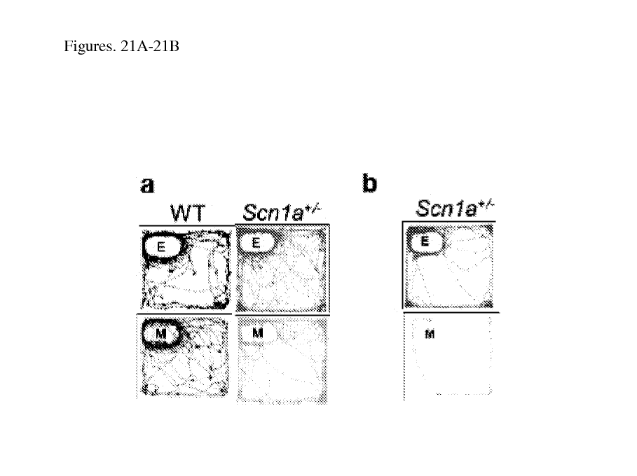

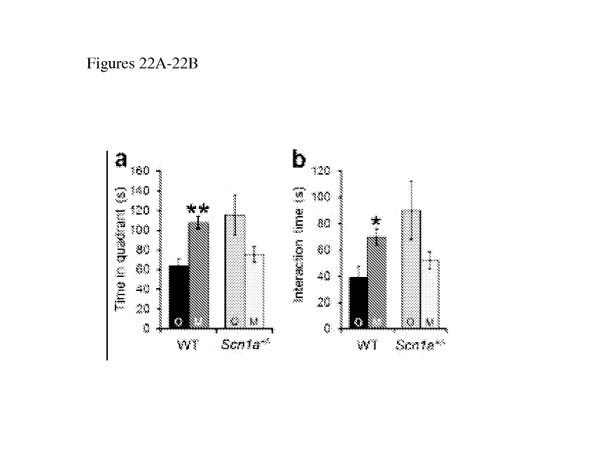

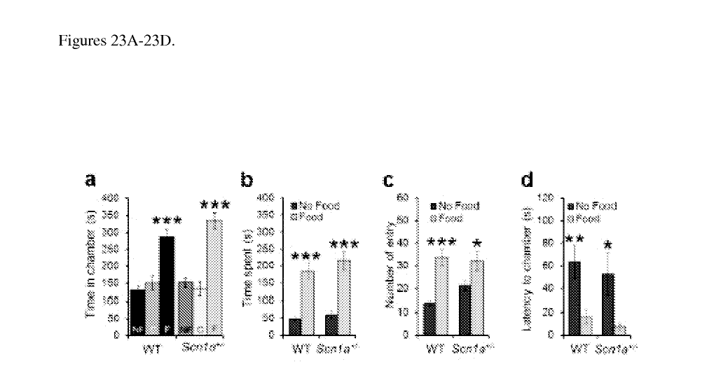

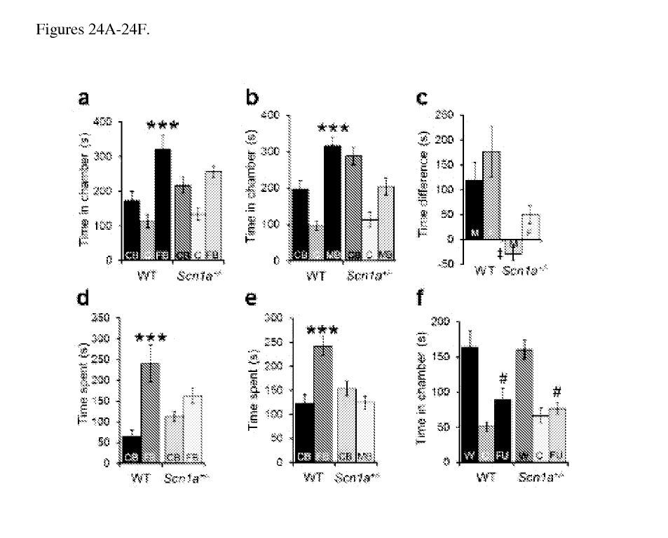

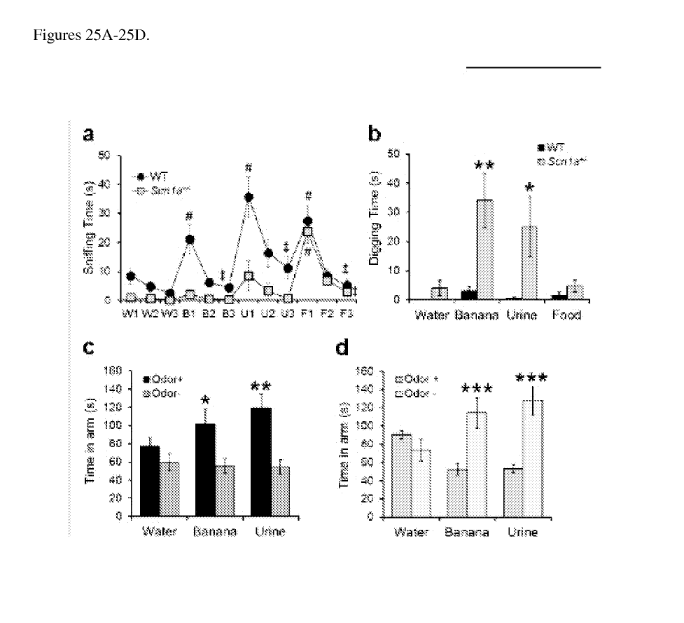

In nocturnal rodents, social interaction and olfactory perception are tightly associated³², and impairment of olfactory perception leads to decreased social interaction³³. The inventors assessed olfaction in modified three-chamber experiments in which a tightly sealed Petri dish containing food pellets and an identical one with holes were placed in the side chambers. Both Scn1a+/− and wild-type mice spent more time in the food-odor chamber, showed a shorter latency to enter it, and entered it more frequently than the odorless chamber (FIGS. 23A-23D). Alternatively, bedding from male or female cages was used as a social odor. Wild-type mice had a strong preference for the chamber containing bedding, whereas Scn1a+/− mice had no preference for these social odors (FIGS. 24A, 24B, 24D, and 24E). In close-interaction analysis, Scn1a+/− mice avoided interacting with male social cues (FIG. 24C), and both wild-type and Scn1a+/− mice strongly avoided fox urine (FIG. 24F). Wild-type mice exhibited strong habituation and dishabituation to odors of banana, male urine and standard food, whereas Scn1a+/− mice gave a normal response to standard food but failed to show habituation/dishabituation to banana or male urine (FIG. 25A). However, Scn1a+/− mice had greatly increased digging behavior when banana and male urine odors were presented, indicating that they detect these odors (FIG. 25B).

Moreover, in a Y-maze olfactory choice test, Scn1a+/− mice strongly avoided banana and male urine, whereas wild-type mice had a strong preference for both (FIG. 25C, 25D). These data indicate that Scn1a+/− mice perceive food odors and social olfactory cues, but they have no interest or avoid unfamiliar odors and social odors. These results further establish a deficit in social interaction^{34,35} and avoidance of environmental change³⁶ in Scn1a+/− mice, as in ASDs.

Scn1a+/− Mice have Deficits in Context-Dependent Spatial Memory

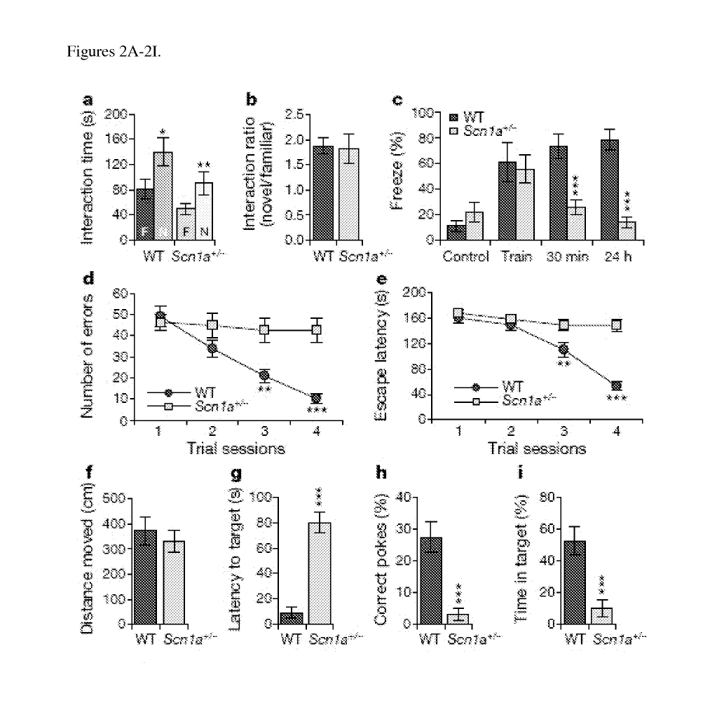

Both wild-type and Scn1a+/− mice had similar ability to recognize a new object 24 h after training (FIGS. 2A, 2B). In the context-dependent fear-conditioning test, Scn1a+/− and wild-type mice showed no freezing behavior during the habituation period in context, and both of them had similar freezing behavior immediately after a mild foot shock (FIG. 2C). However, whereas wild-type mice showed sustained freezing behaviors when returned to the shock cage 30 min and 24 h later, Scn1a+/− mice had substantially reduced freezing behavior (FIG. 2C). The loss of fear-associated freezing behavior was specific because measurements of distance and velocity of movement during the fear-conditioning test did not reveal other fear-associated responses such as panic fleeing (FIG. 26).

To assess spatial learning and memory in the absence of fear, the inventors performed the Barnes circular maze test in which mice learn to rapidly escape a brightly lighted circular field by finding a specific dark hole at its periphery. Scn1a+/− mice failed to improve their learning performance during four days of training (FIGS. 2D, 2E), and had substantially reduced spatial memory during the probe trials at day 5 (FIGS. 2F-2I). These data, together with the results of the context dependent fear-conditioning test (FIG. 2C), indicate that Scn1a+/− mice have severely impaired spatial learning and memory.

Conditional Scn1a+/− Mutant Mice Exhibit Autism-Related Behaviors

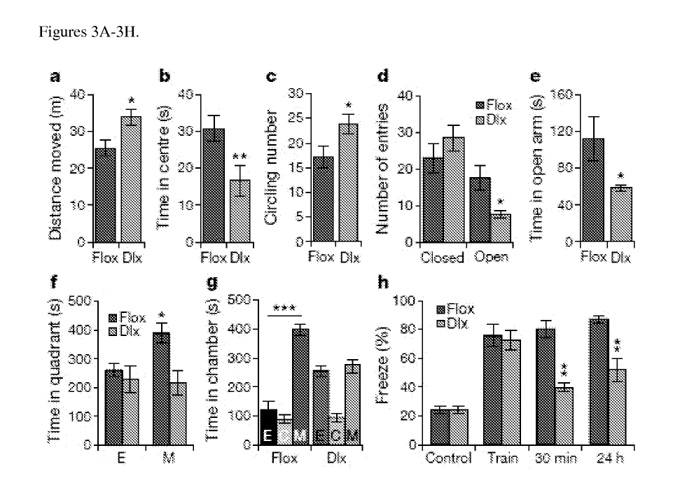

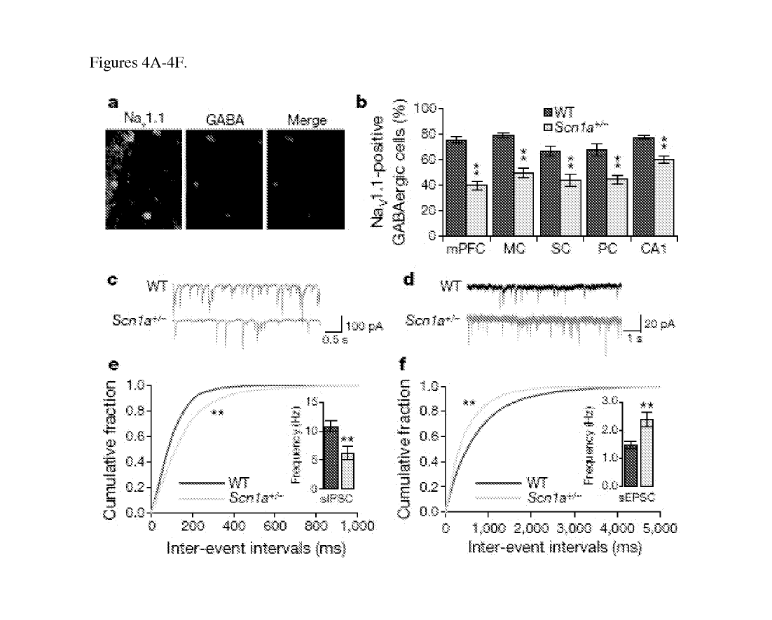

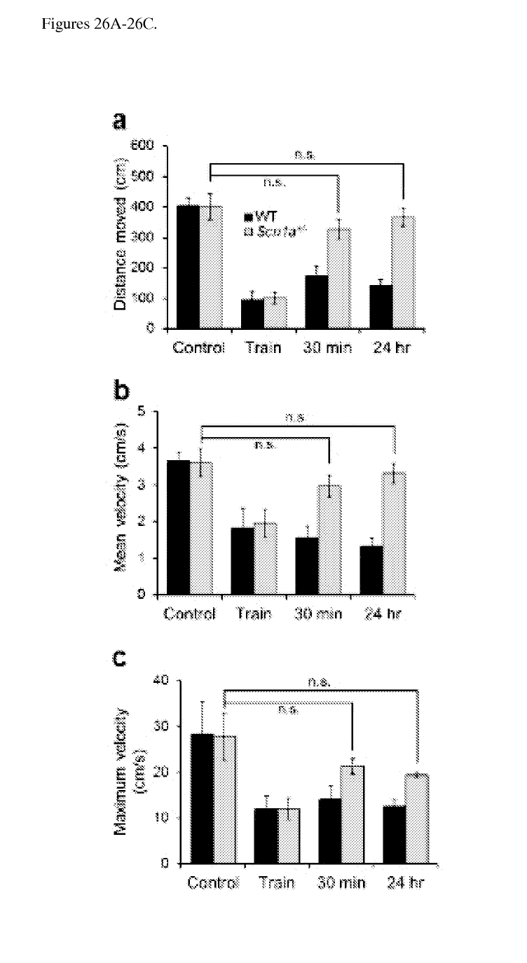

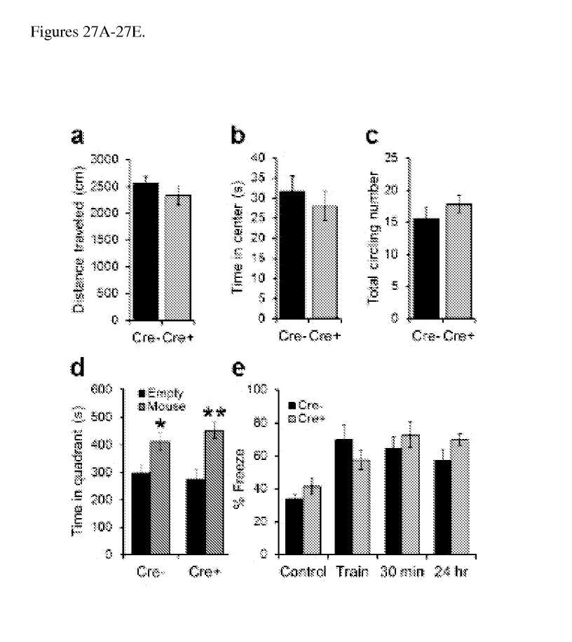

To determine whether the autism-related phenotypes of Scn1a+/− mice emerge specifically from reduced NaV1.1 activity in forebrain GABAergic neurons, the inventors generated forebrain GABAergic neuron-specific conditional Scn1a+/− mutant mice using the DlxI12b-Cre Cre-recombinase mouse line (Dlx1/2-Cre^{17,37,38}). These mice have a specific reduction of NaV1.1 channels in forebrain GABAergic neurons and have similar epilepsy and premature death as Scn1a+/− mice¹⁷. Dlx1/2 Scn1a heterozygous mutant mice (Dlx1/2-Scn1a+/−) recapitulated the autism-related phenotypes and spatial learning deficit of Scn1a+/− mice (FIG. 3), whereas control Cre-positive Scn1a+/+ mice did not (FIG. 27). In the open field test, Dlx1/2-Scn1a+/− mice traveled more (FIG. 3A), spent less time in the center (FIG. 3B), and showed increased circling behavior (FIG. 3C)¹⁷ when compared with Cre-negative Scn1a1/LoxP mice. In the elevated plus maze test, Dlx1/2-Scn1a+/− mice entered the open arms less frequently and spent less time in open arms than Cre-negative Scn1a1/LoxP mice (FIGS. 3D, 3E). In the open field social interaction test, Dlx1/2-Scn1a+/− mice spent less time interacting with the caged stranger mouse compared to Cre-negative Scn1a1/LoxP mice (FIG. 3F). In addition, in the three-chamber social preference test Cre-negative Scn1a1/loxp mice stayed longer in the mouse chamber versus the inanimate-object chamber; in contrast, Dlx1/2-Scn1a+/− mice showed no preference (FIG. 3G). Finally, in the contextual fear-conditioning test, Dlx1/2-Scn1a+/− mice showed similar freezing behavior in control and training sessions, but significantly less freezing behavior in the 30 min and 24 h after the training compared with Scn1a1/loxp mice (FIG. 3H). These results show that Dlx1/2-Scn1a+/− mice reproduce hyperactive and anxiety-like behaviors, deficits in social interactions, and impaired context-dependent fear conditioning of global Scn1a+/− mice. This evidence indicates that the autism-related phenotype emerges from reduced NaV1.1 activity specifically within forebrain GABAergic interneurons.

### Deficit of NaV1.1 Channels Impairs GABAergic Neurotransmission

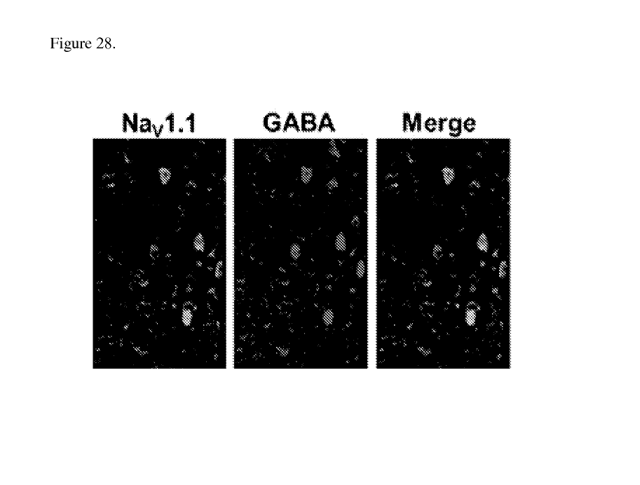

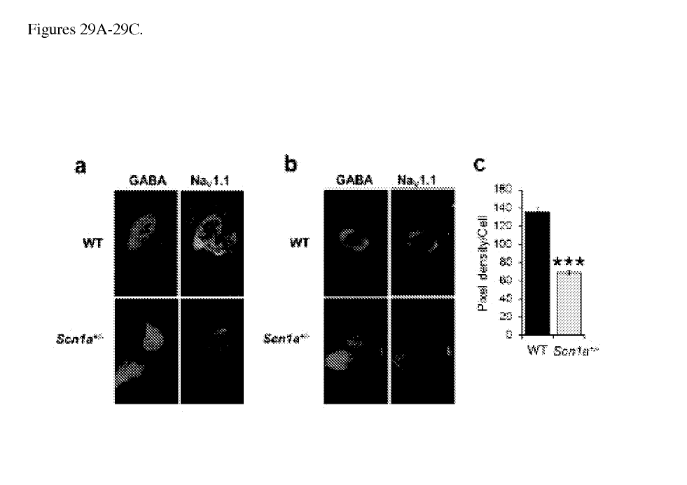

To test the hypothesis that the autism-related phenotypes and spatial learning deficits in Scn1a+/− mice are caused by decreased NaV1.1 activity in GABAergic interneurons in the forebrain, the inventors compared the properties of cortical and hippocampal GABAergic interneurons in wild-type and Scn1a+/− mice. NaV1.1 protein is expressed in adult hippocampal and neocortical interneurons, as assessed by co-immunolabelling of NaV1.1 channels and GABA in the hippocampal CA1 region (FIG. 4A) and prefrontal cortex (FIG. 28). The proportion of GABAergic interneurons expressing a detectable level of NaV1.1 in of Scn1a+/− mice was decreased 20-50% throughout the cortex and hippocampus (FIG. 4B), whereas there was no reduction in the total number of GABA-stained interneurons (FIG. 29, legend). The deep layer of prefrontal cortex was the most affected by the Scn1a mutation (FIG. 4B), and the intensity of immunostaining for NaV1.1 in GABAergic cells with detectable staining was reduced by 50% in the prefrontal cortex (FIG. 29). Some forms of autism are postulated to be caused by an imbalance of synaptic transmission between excitatory and inhibitory circuits²⁶⁻²⁹.

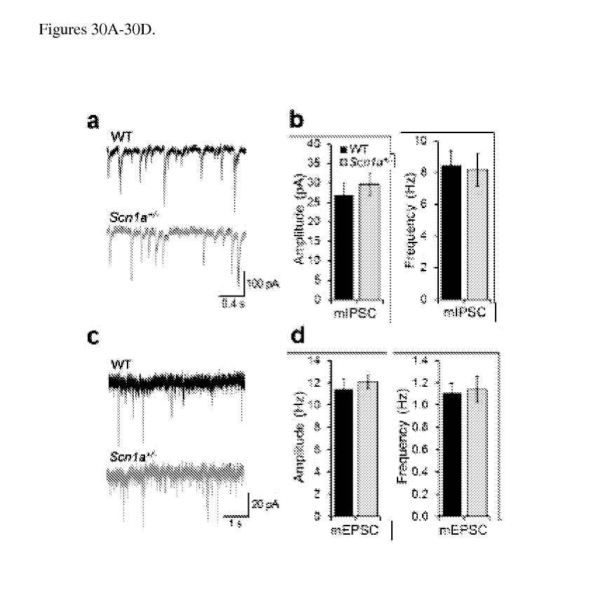

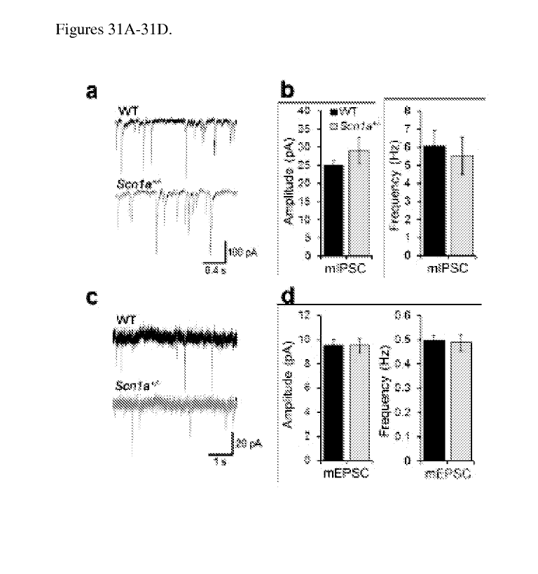

Scn1a+/− mice have reduced sodium currents and impaired action potential firing in both hippocampal interneurons and cerebellar Purkinje neurons^{9,10}, which are GABAergic neurons. When action potentials were blocked with tetrodotoxin (TTX, 1 μM), recordings of miniature inhibitory postsynaptic currents (IPSC) and miniature excitatory postsynaptic current (EPSC) from the hippocampal CA1 region and the prefrontal cortex showed that amplitude and frequency were not altered, indicating normal synaptic function in Scn1a+/− slices (FIGS. 30 and 31). Similarly, in the absence of TTX, the amplitudes of spontaneous IPSCs and spontaneous EPSCs were unchanged (FIGS. 4C. 4D, 32, 33), indicating that the postsynaptic response to released neurotransmitter was not altered.

In contrast, in the absence of TTX, the frequency of spontaneous IPSCs in hippocampal CA1 and prefrontal cortex slices from Scn1a+/− mice was reduced (FIGS. 4C, 4E, 33A, 33B), and the frequency of spontaneous EPSCs was increased (FIGS. 4D, 4F, 33C, 33D) compared to wild-type slices. Because no differences in frequencies of miniature IPSCs or EPSCs were observed when action potentials were blocked by TTX, these changes in frequencies of IPSCs and EPSCs recorded in the absence of TTX must represent differences in action potential-dependent neurotransmission. Therefore, these results indicate that inhibitory synaptic input was decreased because of reduced firing frequency of GABAergic interneurons caused by Scn1a haploinsufficiency, whereas excitatory synaptic activity was increased as an indirect consequence of decreased inhibition.

Treatment of Autism-Related Phenotypes in Scn1a+/− Mice with CLZ

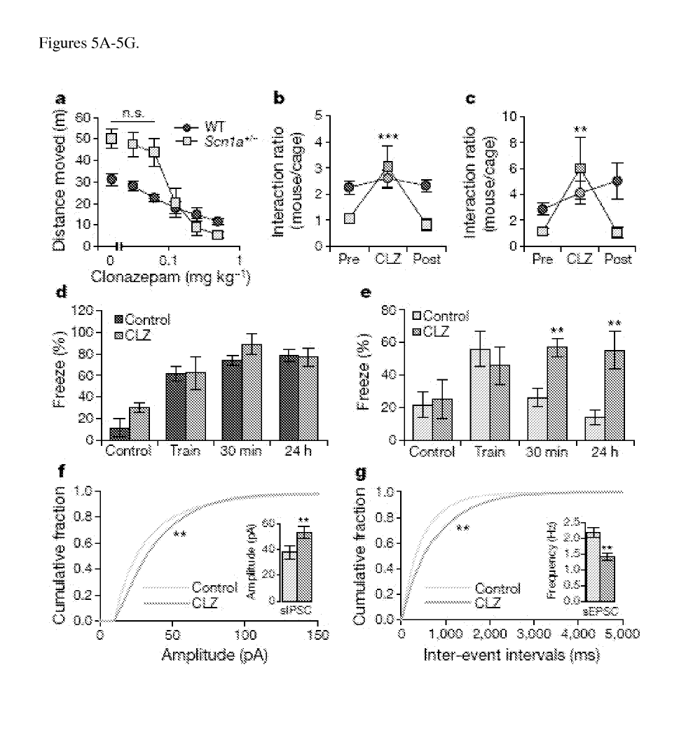

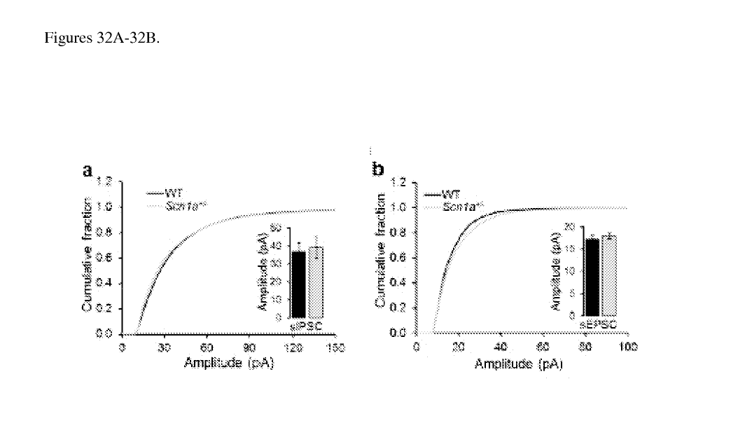

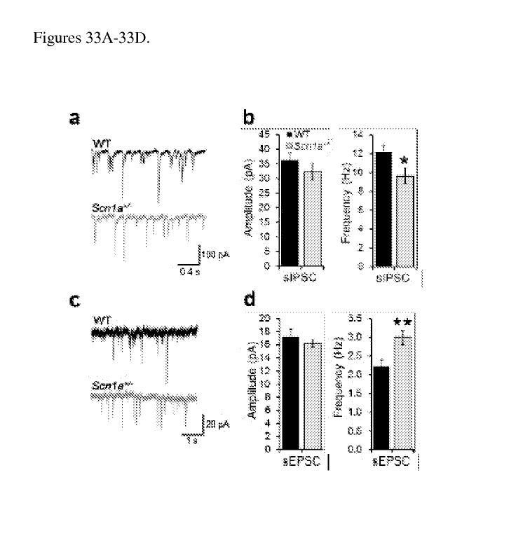

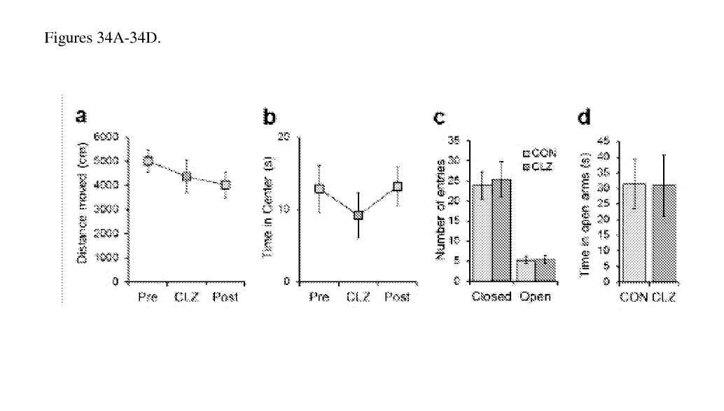

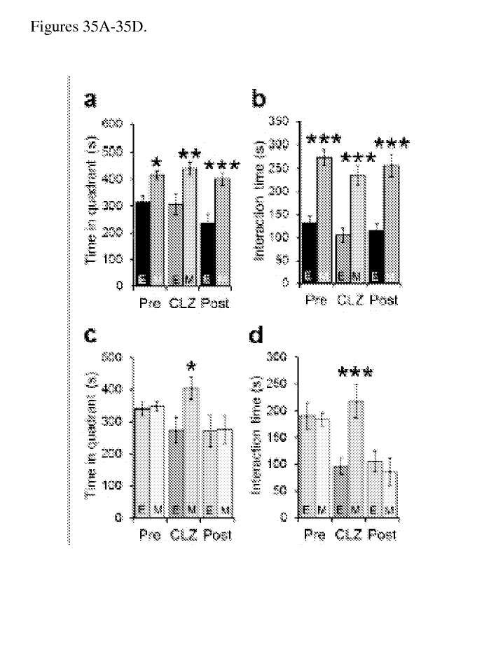

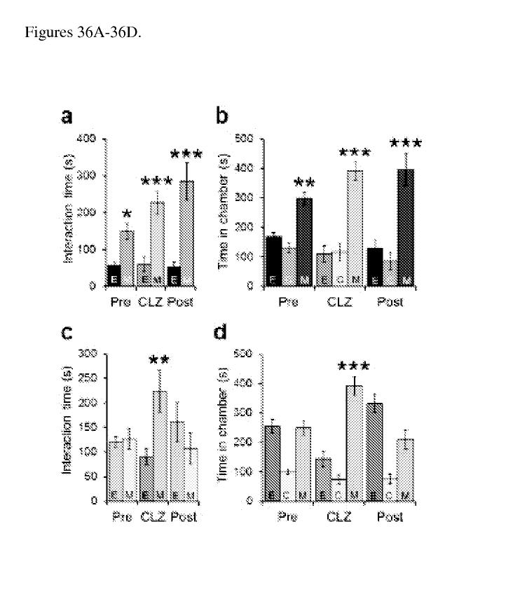

Given that the autism-related phenotype and spatial-learning deficit in Scn1a+/− mice emerge from decreased NaV1.1 activity in GABAergic interneurons, the inventors reasoned that they could be rescued by increasing the strength of GABAergic transmission. To test this idea, the inventors treated Scn1a+/− and wild-type mice with the benzodiazepine CLZ, a positive allosteric modulator of the GABA-A receptor. Benzodiazepines do not open the GABA-A receptor chloride channel in the absence of GABA, but instead boost GABA signaling only when presynaptically released GABA binds to the receptor³⁹. First, the inventors examined the effects of CLZ in the open-field and elevated plus-maze tests to avoid potential sedative and anxiolytic effects in their behavioral experiments, which depend on locomotor activity. The maximal intraperitoneal dose of CLZ that did not cause significant sedation or anxiolytic effect in the open field and elevated plus maze tests was 0.0625 mg/kg for Scn1a+/− mice (FIGS. 5A, 34), 20-fold lower than typical anxiolytic doses⁴⁰. To test the effect of CLZ on social behavior, the inventors performed three sets of identical trials at one-week intervals with the same groups of mice. In the first trial, the inventors performed the social interaction test in the open arena and the three-chamber test without any treatment. In a subsequent trial, the same behavioral tests were performed 30 min after intraperitoneal injection of 0.0625 mg/kg CLZ. In the last trial, the tests were performed 30 min after intraperitoneal injection of vehicle. The data were analyzed as the ratio of the time of interaction with a stranger mouse over the time of interaction with an empty cage. Both in the open arena and in the three-chamber test, CLZ treatment completely rescued impaired social behaviors of the Scn1a+/− mice, and this effect was reversed after the one-week clearing period (FIGS. 5B, 5C, 35, 36). In contrast, low-dose CLZ had no effect on the social behavior of wild-type mice. Treatment with low-dose CLZ 30 min before testing also rescued impaired context-dependent fear conditioning. Whereas wild-type mice were unaffected by CLZ (FIG. 5D), Scn1a+/− mice showed a complete reversal of the loss of their 30-min and 24-h contextual fear memory (FIG. 5E). These results indicate that a single low dose of CLZ can reversibly rescue core autistic traits and cognitive deficit in Scn1a+/− mice.

The inventors also tested the effects of CLZ on GABAergic inhibitory transmission in the hippocampal CA1 region in Scn1a+/− mice. As expected, treatment with 10 μM CLZ increased sIPSC amplitude, but not frequency, in Scn1a+/− hippocampal slices (FIGS. 5F, 32A). The increased amplitude of spontaneous IPSCs after treatment with 10 μM CLZ leads to a decrease in frequency of spontaneous EPSCs, without change in amplitude in Scn1a+/− hippocampal slices (FIGS. 5G, 32B). Similar results were obtained with brain slices from the cerebral cortex (FIG. 33). These results support the hypothesis that behavioral rescue by treatment with CLZ is associated with increased strength of inhibitory transmission.

Despite their adverse impacts on quality of life, the neuropsychiatric co-morbidities and cognitive deficit in DS have not previously been studied in an animal model, and the role of the NaV1.1 channel in these deficits in brain functions was unknown. The results show that mice with heterozygous loss-of-function mutation in NaV1.1 channels have both cognitive deficits and autistic traits, including hyperactivity, anxiety, excessive stereotyped behaviors and social interaction deficits. Together with previously reported phenotypes of epilepsy⁹, premature death⁹, thermally induced seizures¹¹, ataxia¹⁰, and sleep dysfunction¹², these studies demonstrate that Scn1a+/− mice phenocopy all the major symptoms of DS.

These cognitive and behavioral deficits in Scn1a+/− mice are caused by decreased action potential firing in forebrain GABAergic interneurons. The inventors' previous studies indicated that deletion of NaV1.1 channels causes selective reduction in Na1 currents and action potential firing of GABAergic interneurons in hippocampus and cerebellum^{9,10}. This deficit in action potential firing in interneurons in the hippocampus leads to a selective loss of inhibitory neurotransmission compared to excitatory transmission (FIG. 4). Moreover, selective deletion of NaV1.1 channels in forebrain GABAergic interneurons reproduces the core autistic features and cognitive deficits (FIG. 3). These results indicate that the autism-related traits in DS mice are caused by decreased inhibitory neurotransmission in GABAergic interneurons as a consequence of Scn1a haploinsufficiency.

To test this hypothesis further, the inventors treated Scn1a+/− with CLZ, a benzodiazepine, to reverse decreased GABAergic tone. High-dose benzodiazepine has been widely used to alleviate epileptic seizure¹³ and anxiety-like behaviors⁴⁰, but not for rescuing major autism-related behaviors because of its sedative effects. Remarkably, a single low-dose CLZ injection completely rescued deficits in social interactions and fear-associated contextual memory without sedative or anxiolytic effects in Scn1a+/− mice (FIG. 34). Treatment with CLZ also increased spontaneous inhibitory neurotransmission and decreased spontaneous excitatory neurotransmission in hippocampal slices (FIG. 5F, 5G). The reversible rescue of cognitive deficit and autism-related behaviors by CLZ at the time of training implies that these co-morbidities in DS mice are not caused by recurrent seizure-induced excitotoxicity, but instead are caused by Scn1a haploinsufficiency and the resulting reduction of GABAergic transmission. These results indicate that low-dose benzodiazepine treatment could be a potential pharmacological intervention for cognitive deficit and autistic symptoms in DS patients. Genome-wide association studies identified the chromosome 2q24³ region, where the SCN1A gene is located, as an autism susceptibility locus^{18,19}. Sequencing of the genomes of autistic patients identified mutations of SCN1A gene in familial autism²⁰. Exome sequencing revealed that de novo mutations in the SCN1A gene cause autism²¹. These results indicate that DS should be included in the category of ASD-related syndromes, such as Fragile-X syndrome, Rett syndrome and Timothy syndrome⁴¹. With a prevalence of 1:20,000 births for DS and related SCN1A channelopathies⁴², DS is less frequent than Fragile-X syndrome (1:5,000) or Rett syndrome (1:10,000), but much more common than Timothy syndrome (1:1,000,000). Interestingly, mutations in many ASD susceptibility genes also exhibit cytogenetic dysfunctions in GABAergic interneurons^{24-29,43,44}. Thus, autistic traits in DS and in a broad range of ASDs may be caused by a reduction of GABAergic signaling. These results indicate that low-dose benzodiazepine treatment can be effective in alleviating these autistic traits and cognitive impairment in DS and possibly in ASDs more broadly.

### Example 2

### Treatment of Cognitive Deficit and Autism-Spectrum Behaviors in a Mouse Model of Idiopathic Autism

Some strains of inbred mice have spontaneous autism-spectrum behaviors that have arisen without any known specific genetic deficit. These strains of mice are likely to have accumulated multiple spontaneous genetic changes that lead to autism-spectrum behaviors⁷¹, much like the appearance of idiopathic autism in humans. The BTBR mouse line is widely accepted as a model of autism-spectrum behaviors. The inventors have studied the effects of low-dose CLZ treatment on autism-spectrum behaviors in these mice in order to determine whether this drug treatment would be effective in a polygenic, spontaneous form of idiopathic autism. Details of our methods can be found in Han et al.⁷²

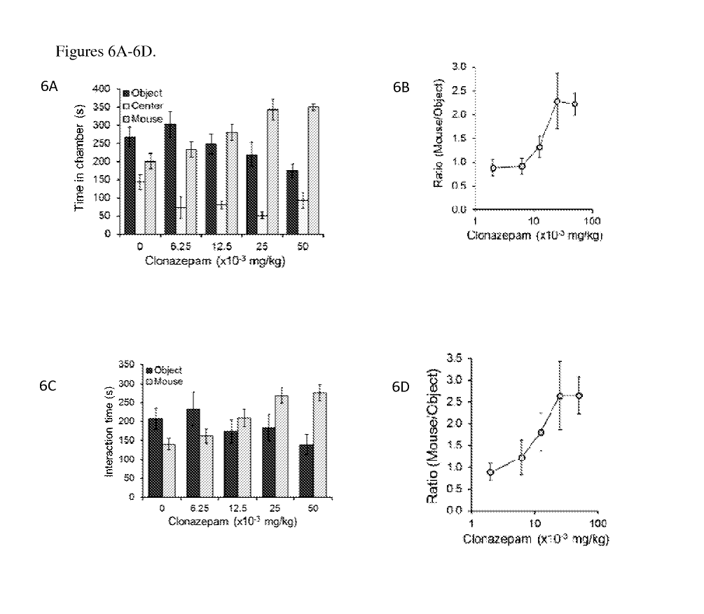

### Low Doses of CLZ Increase Social Interaction of BTBR Mice in the Three-Chamber Test (FIG. 6).

In the three-chamber test, a test mouse is placed in the center chamber, and the preference of the test mouse for interaction with an inanimate object (a small empty cage) in the left chamber vs. a stranger mouse in an identical cage in the right chamber using a digital camera and quantitative video-tracking analysis. Administration of CLZ in doses from 0.00625 mg/kg to 0.05 mg/kg increases the time the test mouse spends in the chamber containing the stranger mouse approximately 2.5-fold (FIG. 6A, 6B) and increases the ratio of time spent interacting with the mouse vs. the object from approximately 1.0 to 2.5 (FIG. 6C, 6D).

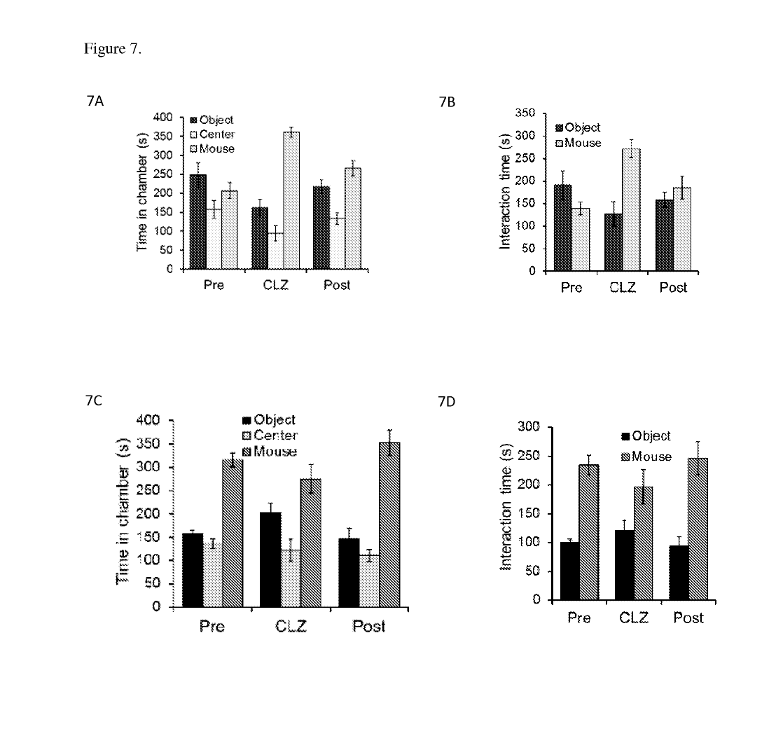

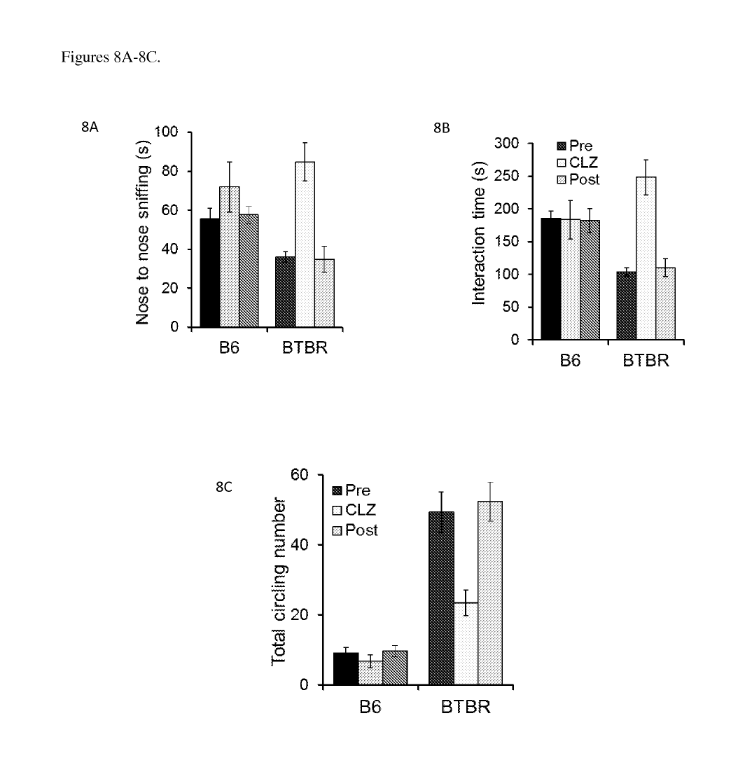

The effects of CLZ are reversible, as shown in a three-week behavioral protocol (FIG. 7A, 7B). On Day 0, untreated BTBR mice do not prefer to interact with a mouse vs. an object. On Day 7, 30 min after treatment with CLZ, BTBR mice recover their preference for interaction with a stranger mouse vs. an object. On Day 14, the effect of CLZ is washed out, as expected from its half-life in vivo of 12 hr, and BTBR mice again do not prefer to interact with the stranger mouse vs. the object. In contrast to these strong drug effects on BTBR mice, low-dose CLZ treatment does not significantly alter the behavior of control C57Bl/6 mice (FIGS. 7C, 7D).

Treatment with CLZ is also effective in rescuing normal social and stereotyped behaviors of BTBR mice in an open field (FIG. 8). Quantitative analysis of video recordings at one-week intervals shows that CLZ reversibly increases nose-to-nose interactions and total interactions of test BTBR mice with a stranger mouse and reduces their stereotyped circling behavior. The same treatment has no significant effect on wild-type C57/Bl16 (B6) mice.

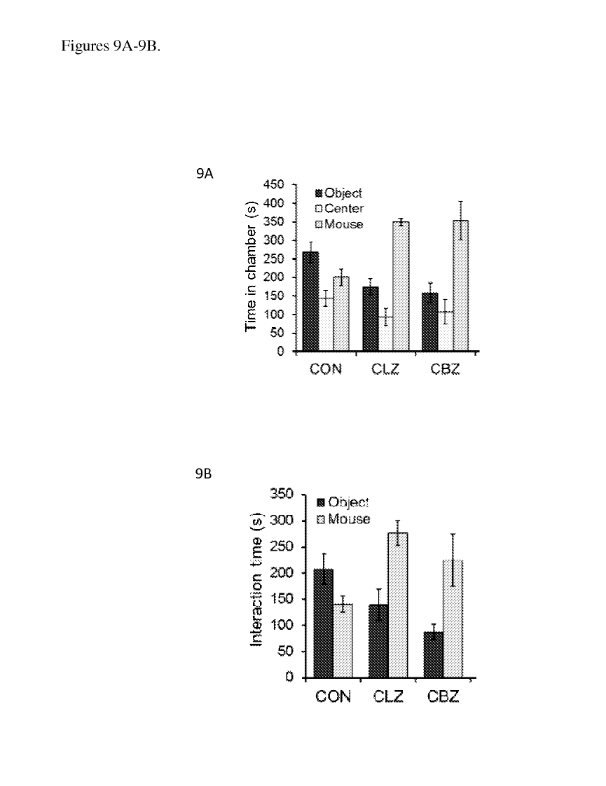

The atypical benzodiazepine clobazam (CBZ) also increases social interaction of BTBR mice in the three-chamber test but has no effect on control mice (FIG. 9). Like CLZ, CBZ increased the time in the chamber with the stranger mouse and the time of interaction with the stranger mouse.

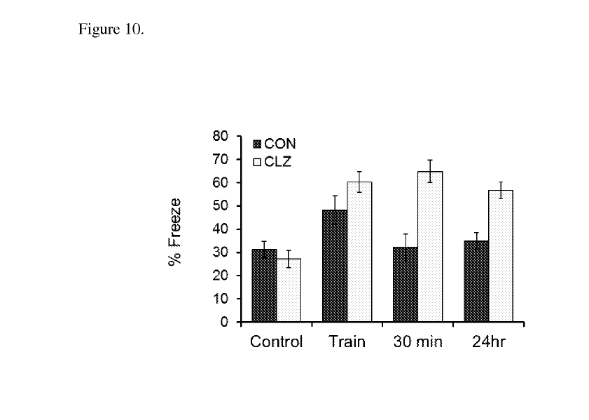

BTBR mice have a cognitive deficit in the context-dependent fear conditioning protocol, which measures the ability of a mouse to recognize a fearful situation, encode the spatial context in which it occurred, and remember that spatial context⁶¹. Treatment with low-dose CLZ rescued their spatial learning in this experimental paradigm. After delivery of a mild foot shock, BTBR mice increased their fear-induced freezing behavior like wild-type mice (FIG. 10). However, whereas wild-type mice fully retained their freezing response at 30 min and 24 h after training, BTBR mice did not remember the spatial context in which they had been shocked (FIG. 10).

Treatment of Dravet Syndrome and Autism with Subunit-Selective GABA-A Receptor Positive Allosteric Modulators

GABA-A receptors are composed of pentameric complexes of α, β, and either γ or δ subunits. Subsynaptic receptors have γ subunits, whereas extrasynaptic receptors have primarily δ subunits. Traditional benzodiazepines bind to a site formed at the interface of α and γ subunits, and they are inactive at extrasynaptic receptors having δ subunits. Five α subunits are known, and they have differential roles in the pharmacological effects of benzodiazepines. α1 is the primary target for sedation, as highlighted by the α1-specific sleep aid zolpidem (AMBIEN™) and the loss of sedative effects in α1 KO mice^{45,46}. These α1-specific effects exhibit rapid tolerance⁴⁷. Drugs acting at α2 and α3 subunits are under development for anxiety, chronic pain, and other indications, and they do not have sedative effects or induce tolerance^{48,49}. Mice lacking α5 subunits have altered performance in spatial learning and memory^{50,51}. Drugs acting at α5 subunits are under development for cognitive enhancement and psychiatric diseases⁵¹, and evidence suggests that α5 subunits are required for tolerance to sedative effects of classical benzodiazepines.⁵² The anti-epileptic effects of benzodiazepines are partially dependent on GABA-A receptors with α1 subunits, but other subunits are also involved⁵². The next-generation subunit-specific positive allosteric modulators of GABA-A receptors are of great interest as treatments for DS, because they may prevent seizures and improve cognition without sedative side effects and without tolerance. In some cases, these drugs may act at binding sites with different properties from the benzodiazepine site. They are also of great interest for treatment of autism-spectrum behaviors because they may enhance inhibitory neurotransmission without sedative side effects.

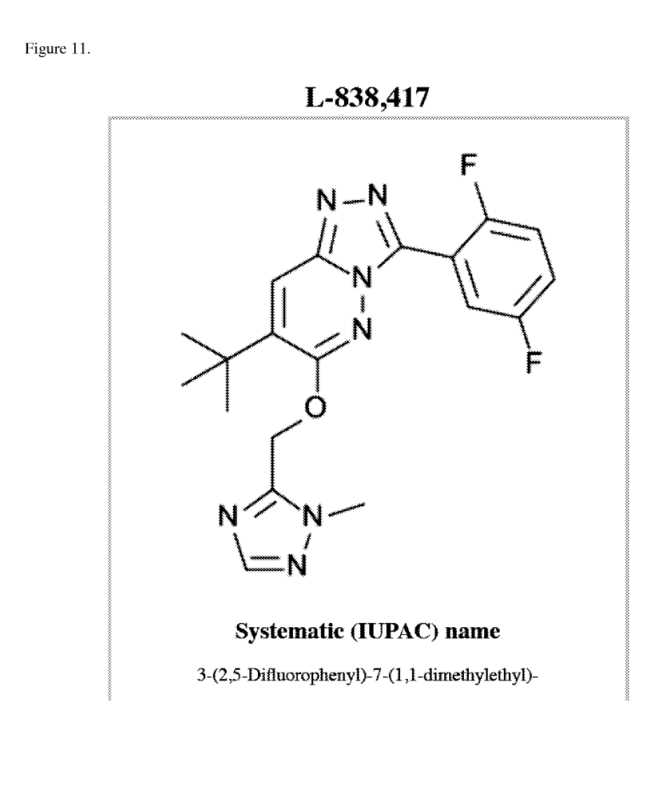

L-838,417 is an anxiolytic drug used in scientific research (FIG. 11). It has similar effects to benzodiazepine drugs, but is structurally distinct and so is classed as a non-benzodiazepine anxiolytic. The compound was developed by MERCK, SHARP & DOHME™. L-838,417 is a subtype-selective GABA-A activator, acting as a partial agonist at α2, α3 and α5 subtypes, but as an antagonist at the α1 subtype, and it has little affinity for the α4 or α6 subtypes.⁵³ This gives it selective anxiolytic effects, which are mediated mainly by α2 and α3 subtypes, but with little sedative or amnestic effects as these effects are mediated by α1.^{54,55} Some sedation can still be expected due to its activity at the α5 subunits, which can also cause sedation; however no sedative effects were seen in animal studies even at high doses, indicating that L-838,417 is primarily acting at α2 and α3 subtypes with the α5 subtype of lesser importance.^{56,57} As might be predicted from its binding profile, L-838,417 substitutes for the anxiolytic benzodiazepine chlordiazepoxide in animals, but not for the hypnotic imidazopyridine drug zolpidem.^{58,59} The synthesis of L-838,417 and similar compounds was described in 2005 in the Journal of Medicinal Chemistry.⁶⁰

Rescue of Cognitive Deficit and Autistic-Like Behaviors with GABA-A Enhancing Drugs Specific for α2 and α3 Subunits

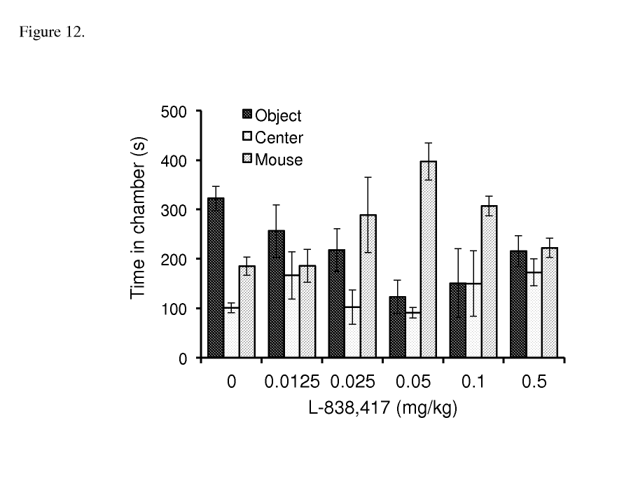

In order to test the effectiveness of α2,3-specific GABA-A receptor positive allosteric modifiers, the inventors used the selective compound L838,417, which is available commercially. In the three-chamber test of social interaction, WT mice prefer to interact with a stranger mouse rather than with an inanimate object (a small mouse cage; FIG. 12), with approximately 2.5-fold greater interaction time with the stranger mouse (see above and reference number 61 for methods). In contrast, untreated BTBR mice do not prefer to interact with a stranger mouse in preference to an inanimate object (FIG. 12). Increasing doses of L818,417 progressively increase the time of interaction with the stranger mouse and decrease the time of interaction with the inanimate object (FIG. 12). Maximum effect is achieved at a dose of 0.05 mg/kg, and larger doses cause a reduced response. These results reveal clearly why low-dose treatment is required for beneficial effects on social interaction. A likely interpretation of this biphasic dose-response curve is that low doses of L818,417 interact preferentially with a subset of GABA-A receptors containing α2 and/or α3 subunits in neural circuits that are important for normal social behavior, but higher concentrations induce interactions at other GABA-A receptors in other neural circuits that oppose those beneficial effects.

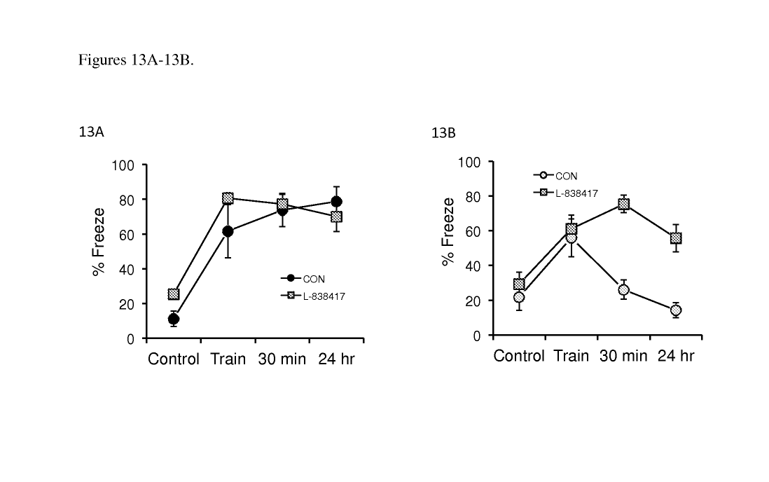

In the context-dependent fear conditioning test of spatial learning and memory, the control C57BL/6 mice retain their memory of the spatial context of a fearful event (a mild footshock) for 24 Hr, and drug treatment had no effect (FIG. 13A). In contrast, BTBR mice lose their memory of the spatial context in which they have experienced fear caused by a foot shock when tested 30 min and 24 h later (FIG. 13B). Administration of L838,417 at a dose of 0.05 mg/kg 30 min before the training session and the 24-h memory session substantially improved the memory of the spatial context in which the fearful event (the footshock) had occurred (FIG. 13B). These results show that a dose of L838,417 that gives optimal rescue of deficits in social behavior (see above) also improves spatial learning and memory in BTBR mice.

### Example 3

### Exemplary Experimental Procedures

### Mice

Scn1a mutant mice were generated by targeted deletion of the last exon encoding domain IV from the S3 to S6 segment and the entire C-terminal tail of NaV1.1 channels as described previously⁹. Mutant mice were generated on a congenic 129/SvJ background and backcrossed to C57Bl/6J background to at least the F10 generation. The animals used in this study were generated by crossing heterozygous mutant males of C57Bl/6J background with WT C57Bl/6J females; from this cross wildtype and heterozygote mice were born in 1:1 ratio. Mice were genotyped as described previouslyl¹⁰. Floxed Scn1a heterozygous mutant mice were generated by replacing the endogenous exon-25 of Scn1a with a targeting vector containing the exon flanked by LoxP sites and an FRT-flanked neomycin-selection cassette, which was removed prior to experiments. Floxed Scn1a homozygous (LoxP/LoxP) and heterozygous (+/LoxP) mice maintained on a C57Bl/6J background were indistinguishable from WT littermates. In the Dlx1/2-I12b-Cre transgenic mouse, an intergenic Dlx1 and Dlx2 enhancer drives expression of Cre recombinase specifically in forebrain GABAergic neurons, including interneurons in cerebral cortex and hippocampus. All behavioral tests were done with age-matched littermate pairs of male mice, aged 6 to 10 months. All experiments with animals were performed according to the National Institutes of Health Guide for Care and Use of Laboratory Animals and were approved by the University of Washington Institutional Animal Care and Use Committee.

### Immunocytochemistry

An antibody that specifically recognizes the α1 subunits of Type I sodium channels (anti-NaV1.1, rabbit polyclonal) was used in this study to detect differences in expression levels in WT and Scn1a+/− mice. This antibody was generated against residues 465-481 of NaV1.1. The generation, purification, and characterization of these antibodies have been reported previously⁶². An anti-GABA (gamma-amino butyric acid, guinea pig polyclonal) antibody was obtained from ABCAM™ (Cambridge, Mass.) and used as a marker for GABAergic interneurons. Adult mice were anesthetized with isoflurane and intracardially perfused with 4% paraformaldehyde, and the brain was removed immediately. The tissue was postfixed for 2 h, successively placed in 10% and 30% sucrose in 0.1 M phosphate buffer (PB) overnight for each sucrose solution, cut on a sliding microtome (40 μm), and stored in 0.1 M PB containing 0.02% sodium azide. Free-floating sections were then processed for immunocytochemistry. Briefly, the tissue was rinsed in 0.1 M Tris buffer (TB) for 15 min, and rinsed in 0.1 M Tris buffered saline (TBS) for 15 min. For double labeling, the tissue was then incubated in affinity-purified anti-NaV1.1 antibody (diluted 1:150) and anti-GABA (diluted 1:600) in TBS containing 0.1% Triton X-100 and 1% normal goat serum for 36 hrs at 4° C. The tissue was then rinsed in TBS for 1 h, incubated in anti-rabbit IgG tagged with Alexa 488 (diluted 1:400, INVITROGEN™) and anti-guinea pig IgG tagged with Alexa 555 (diluted 1:400, INVITROGEN™) for 3 h at 37° C. The tissue was finally rinsed in TBS for 10 min, rinsed in TB for 20 min, mounted on gelatin subbed slides, cover slipped with VECTASHIELD™ (VECTOR™), sealed with nail polish and viewed under the microscope. For controls, the primary antibody was omitted, replaced with normal rabbit serum, or preincubated in the antigenic peptide (15-20 μM) overnight at 4° C. before being applied to the tissue sections. Following completion of staining, digital images were collected on a LEICA™ SL confocal microscope located in the Keck Imaging Facility at the University of Washington. For quantification purposes, tissue slices from 3 WT and 3 Scn1a+/− mice were processed for immunocytochemistry simultaneously and imaged in the same confocal session using the same gain, offset, and laser intensity. A one-in-ten series of sections was analyzed from each animal. The digital images were opened in PHOTOSHOP™ 7 (ADOBE SYSTEMS™) and blind cell counts were made to determine the number of cells double-labeled for NaV1.1 and GABA from various regions of the brain. Intensity of cell staining was analyzed using the Region-of-Interest function in the IGOR PRO™ software (WAVEMETRICS™).

### Open-Field Test

Open field test was performed as previously described²³. Briefly, each individual mouse was placed near the wall-side of 38×42 cm open-field arena, and the movement of the mouse was recorded by USB webcam (LIFECAM™ HD-6000, MICROSOFT™) and PC-based video capture software (WINAVI VIDEO CAPTURE™, ZJMEDIA DIGITAL TECHNOLOGY™) for 10 min. The recorded video file was further analyzed by off-line video tracking software (ETHOVISION™ XT 7.0, NOLDUS TECHNOLOGY™). Total distance traveled and time in center (15×15 cm imaginary square) were measured. The open field arena was cleaned with 70% ethanol and wiped with paper towels between each trial. All data shown are means±s.e.m. and analyzed using Student's two-tailed, unpaired t-test.

### Stereotyped Behavior

During a 10-min open-field test period, the amount of time spent grooming was measured manually, assisted by the manual scoring function in the video tracking software (ETHOVISION™ XT 7.0, NOLDUS TECHNOLOGY™). The observer was blind to the genotype. Circling behavior was scored automatically by the video tracking software (ETHOVISION™ XT 7.0, NOLDUS TECHNOLOGY™). A complete 360-degree turn of nose angle with respect to the body center was counted as one circling event. All data shown are means±s.e.m. and analyzed using Student's two-tailed, unpaired t-test.

### Nesting Behavior

Single-housed mice were transferred into a new cage with nest-building material, a 5×5 cm square of white compressed cotton pads (NESTLETS™; ANCARE™, Bellmore, N.Y.) in a random corner. After 6, 24, and 48 h, nest building was scored on a scale of 0-3, as previously described⁶³. All data shown are means±s.e.m. and analyzed using two-way ANOVA, with time and genotype as levels, using Bonferroni's post hoc comparisons.

### Elevated Plus Maze Test

The elevated plus maze is a plus-shaped maze that is elevated 60 cm above the floor. It consists of two closed arms surrounded by 15-cm high transparent walls and two open arms (5×25 cm) with a small ledge along the side of two open arms to prevent falling from the maze during the test. Each mouse was placed in the center (5×5 cm) of the maze facing one of the closed arms. During the 10-min test period, the movement of the mouse was recorded by a USB webcam (LIFECAM™ HD-6000, MICROSOFT™) and PC-based video capture software (WINAVI VIDEO CAPTURE™, ZJ MEDIA DIGITAL TECHNOLOGY™). The recorded video file was further analyzed by off-line video tracking software (ETHOVISION™ XT 7.0). Times spent in closed arms, center, and open arms were measured. The maze was cleaned with 70% ethanol and wiped with paper towels between each trial. All data shown are means±s.e.m. and analyzed using Student's two-tailed, unpaired t-test.

### Three-Chamber Social Preference Test

The test was performed as described previously³⁴ with minor modifications. The three-chamber apparatus is a non-transparent PLEXIGLAS™ box (25×50 cm) with two transparent partitions that make left, center, and right chambers (25×16.7 cm). Each partition has a square opening (5×5 cm) in the bottom center. A cylindrical wire cage (10.5 cm diameter; GALAXY PENCIL CUP™ SPECTRUM DIVERSIFIED DESIGNS™) was used as an inanimate object or the cage housing a stranger mouse. A cylindrical bottle filled with water was placed on the top of the wire cup to prevent the test mouse from climbing to the top of the cup. The three-chamber unit and wire cups were cleaned with 70% ethanol and wiped with paper towels between each trail. In the first 10-min session, a test mouse was placed in the center of the three-chamber unit, where two empty wire cages were located in the left and right chambers to habituate the test mouse. The mouse was allowed to freely explore each chamber. In the second 10-min session, an age- and gender-matched C57Bl/6J mouse (M1) that had never been exposed to the test mouse, was placed in one of the two wire cages. The wire cage on the other side remained empty (E). Then, the test mouse was placed in the center, and allowed to freely explore the chamber for 10 min. The test mouse was removed and in the last 10-min session, a second age- and gender-matched C57Bl/6J stranger mouse (M2) that had never been exposed to the test mouse, was placed in one wire cage, which previously served as an empty cage. Thus, the test mouse would now have the choice between a mouse that was already familiar (M1) and a new stranger mouse (M2). The test mouse was placed in the center, and allowed to freely explore the chamber for 10 min. The movement of the mouse was recorded by a USB webcam (LIFECAM™ HD-6000, MICROSOFT™) and PC-based video capture software (WINAVI™ Video Capture, ZJ MEDIA DIGITAL TECHNOLOGY™). The recorded video file was further analyzed by off-line video tracking software (ETHOVISION™ XT 7.0). Time spent in each chamber, and time spent within a 5 cm radius proximal to each wire cage, was measured. All data shown are means±s.e.m. and analyzed using two-way ANOVA with Bonferroni's post hoc analysis and one-way ANOVA with Tukey's post hoc analysis.

### Open-Field Social Interaction Test

The open-field social interaction test was performed as described previously⁶⁴ with minor modifications. The test was performed in the open field arena that was used for the open field test. An age- and gender-matched C57Bl/6J mouse (M) caged in a rectangular wire mesh cage (6×6×10 cm), was used as a social cue. The stranger mouse had never been exposed to the test mouse. The test mouse was placed in the open field arena with an empty wire cage for 10 min for habituation. Following the 10 min session, a stranger mouse was placed in the same cage and the test mouse was allowed to explore the arena for another 10 min. The empty cage or caged stranger mouse was placed in the center of one quadrant of the arena, and immobilized on the floor of the arena with double-sided tape. For the social choice test, an inanimate object and the caged stranger mouse were placed simultaneously in the opposite side of the quadrant of the arena, then the test mouse was allowed to choose either an inanimate object or the caged stranger mouse. The movement of the mouse was recorded with a USB webcam (LIFECAM™ HD-6000, MICROSOFT™) and PC-based video-capture software (WINAVI™ Video Capture, ZJ MEDIA DIGITAL TECHNOLOGY™). The recorded video file was further analyzed by off-line video tracking software (ETHOVISION™ XT 7.0). Time spent in the cage-containing quadrant, and time spent in the area 5 cm proximal to the cage were measured. Immobilization behavior was also measured by video tracking software. Immobilization was defined as the time when the mean velocity of a mouse was continuously less than 1 cm/s during a 10-s interval. The open field arena and the cage were cleaned with 70% ethanol and wiped with paper towels between each trial. All data shown are means±s.e.m. and analyzed using two-way ANOVA with Bonferroni's post hoc analysis.

### Reciprocal Interaction Test

A test mouse and an age- and gender-matched stimulus mouse that was marked on the tail using a black permanent marker were introduced in a neutral cage with fresh bedding. The cage was used only once. The mice had not interacted previously. The social interactions of mice were recorded by USB webcam (LIFECAM™ HD-6000, MICROSOFT™) and PC-based video capture software (WINAVI™ Video Capture, ZJ MEDIA DIGITAL TECHNOLOGY™) for 10 min. Time spent in aggressive interactions, such as attacking, wrestling, and biting the dorsal surface, and time spent in non-aggressive interactions, such as nose-to-nose sniffing, anogenital sniffing, and grooming were measured manually using the event-recording function in the video-tracking software by a researcher who was blind to the genotype of test mice. All data shown are means±s.e.m. and analyzed using two-way ANOVA with Bonferroni's post hoc analysis.

### Olfactory Discrimination Tests

The olfactory discrimination ability of mice was examined with the modified three-chamber test. Instead of the wire cage or caged mouse used for social preference test, tightly sealed petri dishes containing food pellets were used as non-social olfactory cues. A dish with holes to release food odor was placed in one chamber, and another dish with no holes was placed in the other chamber. Alternatively, the bedding from 3-day-used male or female cages was used as a social odor instead of food pellets, and clean bedding was used as control, to examine the ability of mice to discriminate social odors. Fox urine was also used as a control, aversive social odor. The three-chamber test apparatus and wire cups were cleaned with 70% ethanol and wiped with paper towels before each trial. A test mouse was placed in the center chamber, and allowed to explore the chambers for 10 min. A Y-maze was used for an olfactory choice test. A Y-shaped maze composed of three equal-sized transparent PLEXIGLAS™ arms (30×20×10 cm) with removable gates controlling entry into each arm was used. Two cotton-tipped swabs, one with odor and the other without odor, were placed at the end of the right and left arms, and the test mouse was placed in the central arm. During a 5-min trial, the test mouse was only allowed to explore the right and left arms by closing the gate of the central arm. The movement of the mouse was recorded by USB webcam (LIFECAM™ HD-6000, MICROSOFT™) and PC-based video capture software (WINAVI™ Video Capture, ZJ MEDIA DIGITAL TECHNOLOGY™). The recorded video file was further analyzed by off-line video tracking software (ETHOVISION™ XT 7.0, NOLDUS TECHNOLOGY™). Time spent in each chamber, time spent in the area 5 cm proximal to each wire cage, the number of entries in each chamber, and the latency to access each chamber were measured. For the Y-maze olfactory choice test, time spent in each arm was measured. All data shown are means±s.e.m. and analyzed using two-way ANOVA with Bonferroni's post hoc analysis and one-way ANOVA with Tukey's post hoc analysis.

### Olfactory Habituation/Dishabituation Test

The test was performed as described previously⁶⁵ with minor modifications. All the tests were done in a home cage, where the test mouse was singly housed. Odor stimulants were delivered with a cotton-tipped swab through a hole in the center of the cage top positioned 7 cm above the bedding. After 30 min of habituation with a cotton-tipped swab without odor stimulant, the test mouse was stimulated by serial application of odorants: Water, Banana flavor 1:100 dilution (KROGER™ Cincinnati, Ohio), C57BL/6J male urine 1:100 dilution, and finely ground food pellets. Each stimulus was 2 min in duration with a 1 min inter-trial interval. The sequence of the odor stimulation was as follows: Water1, Water2, Water3, Banana1, Banana2, Banana3, Urine 1, Urine 2, Urine 3, Food1, Food2, Food3. Time spent sniffing the odorants was measured by manual observation with a stopwatch. Sniffing was only scored when the test mouse's nose was closer than 1 cm from the swab. Digging behavior was also measured by manual observation of the test mouse in response to each odorant during the trial. All data shown are means±s.e.m. and analyzed using two-way ANOVA with Bonferroni's post hoc analysis and one-way ANOVA with Tukey's post hoc analysis.

### Novel Object Recognition Test

The arena used for the novel object recognition test was a rectangular cage (25×50 cm) covered with fresh bedding. The arena was used only once for each mouse. In the habituation session, a test mouse was placed in the arena and allowed to explore for 10 min. Following habituation, two objects of similar size, but different shape and color, were placed in the opposite corners of the arena, 10 cm from the side walls, and then the test mouse was placed in the center of the arena, and allowed to explore the arena including the two novel objects for 10 min. After 24 h, one object was replaced with another novel object, which was of similar size but different shape and color than the previous object. Then, the same test mouse was placed in the center, and allowed to explore the arena and the two objects. The movement of mice was recorded by USB webcam (LIFECAM™ HD-6000, MICROSOFT™) and PC-based video capture software (WINAVI™ Video Capture, ZJ MEDIA DIGITAL TECHNOLOGY™) for 10 min. The recorded video file was further analyzed by off-line video tracking software (ETHOVISION™ XT 7.0, NOLDUS TECHNOLOGY™). Time in each object area (10×10 cm) was measured. All data shown are means±s.e.m. and analyzed using Student's two-tailed, paired t-test.

### Barnes Circular Maze Test

The Barnes circular maze is a planar, round white PLEXIGLAS™ platform (92 cm diameter), 1 m above the floor, with 20 evenly spaced holes (7 cm diameter) located 5 cm from the perimeter. A black escape box (15×7×7 cm) was placed under one hole. Spatial cues with distinct patterns and shapes were placed on the wall of the testing room. A 500 lux light was turned on during the trial. An experimenter remained in the same place with minimal movement throughout the trials. The platform and the escape box were cleaned thoroughly with 70% ethanol and paper towels between each trial to remove olfactory cues. One day before the training trials began, test mice were habituated in the target box for 3 min. The training trials were repeated for 4 consecutive days, and 3 trials were performed each day with 20 min inter-trial intervals. At the beginning of each trial, a test mouse was placed in a cylindrical holding chamber (10 cm diameter) located in the center of the maze. After 10 s of holding time, the mouse was allowed to search for the target hole for 3 min. If the mouse failed to find the target hole in 3 min, it was gently guided into the target hole by the experimenter's hands. When the mouse entered the escape box, the light was turned off and the mouse remained undisturbed for 1 min. The movement of the mouse was recorded, and the number of errors made and the latency to find the target hole were measured during the training trials by video tracking software. On day 5, the probe trial was performed with each mouse. The escape box was removed, and the test mouse was allowed to find the target hole freely for 90 s. During the probe trial, total distance traveled and the latency to find the target hole were measured. % correct pokes and % time in the target area were also measured. All data shown are means±s.e.m. and analyzed using two-way ANOVA with Bonferroni's post hoc analysis and Student's two-tailed, unpaired t-test.

### Contextual Fear-Conditioning Test

The contextual fear-conditioning chamber was a square arena (25×25 cm) with clear PLEXIGLAS™ walls and a metal grid floor connected to a circuit board that delivered shocks to the metal grid (COULBOURN INSTRUMENTS™). An analog camera was attached on top of the chamber. The camera and the circuit board were connected to a personal computer, and its software (FREEZE FRAME™ 2.0, ACTIMETRICS™) controlled the circuit and recorded the data. The chamber was cleaned with 70% ethanol and wiped with paper towels before each session. In the habituation session, a test mouse was placed in the chamber and allowed to explore for 2 min⁶⁶⁻⁶⁹. Immediately after habituation, the test mouse received a single mild foot shock (2 s, 0.5 mA). After staying in the chamber one more min, the mouse was removed from the chamber. For the 30-min short-term memory test, the mouse was returned to the context 30 min after the end of the training session. For 24-hr long-term memory test, the mouse was returned to the context 24 hr after the training session. The movement of mice was also recorded by a USB webcam (LIFECAM™ HD-6000, MICROSOFT™) and PC-based video capture software (WINAVI™ Video Capture, ZJ MEDIA DIGITAL TECHNOLOGY™) for 2 min. The recorded video file was further analyzed by off-line video tracking software (ETHOVISION™ XT 7.0, NOLDUS TECHNOLOGY™). The freezing scores were calculated by dividing the test session into 1 min bouts and averaging together all 2 min for each animal. All data shown.

### Brain Slice Electrophysiology

Hippocampal slices were prepared from P21-P25 mice using standard procedures modified from those previously described⁷⁰. Briefly, mice were deeply anesthetized with isoflurane and decapitated. The brain was quickly removed and horizontal hippocampal slices (400 μm) were cut with a modified vibratome (PELCO™ 101 series 1000; TED PELLA™, Inc., Redding, Calif.) in chilled (0-4° C.) slicing solution containing 75 mM sucrose, 87 mM NaCl, 25 mM NaHCO₃, 25 mM D-glucose, 2.5 mM KCl, 1.25 mM NaH₂PO₄, 0.5 mM CaCl₂, 7.0 mM MgCl₂, and pH 7.3. The slices were transferred to a storage chamber with fresh ACSF containing 126 mM NaCl, 2.5 mM KCl, 2.0 mM MgCl₂, 2.0 mM CaCl₂, 1.25 mM NaH₂PO₄, 26 mM NaHCO₃, and 10 mM D-glucose, pH 7.3, and incubated at 37° C. for 45 min. The slices were then incubated at room temperature for at least another 45 min before recording. All solutions were saturated with 95% O₂ and 5% CO₂. Whole-cell voltage-clamp recordings were performed on CA1 pyramidal neurons within hippocampal slices visualized under differential interference contrast (DIC) optics, and near infrared (bandpass 750-800 nm) illumination was used to identify individual neurons in a recording chamber located on an upright microscope (NIKON ECLIPSE™ E600FW). Patch electrodes were pulled from 1.5 mm outer diameter thin-walled glass capillaries (150E-4; WORLD PRECISION INSTRUMENTS™, Sarasota, Fla.) in three stages on a Flaming-Brown micropipette puller (model P-97; SUTTER INSTRUMENTS™, Novato, Calif.). In the recordings of inhibitory postsynaptic currents (IPSCs), the patch electrodes were filled with intracellular solution containing CsCl (135 mM), N-2-hydroxyethylpiperazine-N′-2-ethanesulfonic acid (HEPES; 20 mM), ethylene glycol-bis(β-aminoethylether)-N,N,N′,N′-tetraacetic acid (EGTA; 2 mM), Mg-ATP (2 mM), Na-GTP (0.5 mM), pH 7.25. Kynurenic acid (KA, 1 mM) was included in the perfusion solutions to block the excitatory synaptic transmission in IPSC recordings. In the recordings of excitatory postsynaptic currents (EPSCs), CsCl was replaced by Cs-methanesulfonate. Where indicated, TTX (1 μM) was applied in the perfusion solutions to block action potentials and allow recording of miniature IPSCs and miniature EPSCs. When filled with intracellular solution, patch electrode resistance ranged from 3 to 5 MΩ. Recordings were obtained through a Multiclamp 700A amplifier (MOLECULAR DEVICES™) by the data-acquisition software (PCLAMP™ 8.0; MOLECULAR DEVICES™). Access resistance was continuously monitored for each cell. Only the cells with access resistance less than 20 MΩ were recorded, and recordings were terminated/discarded when a significant (>10%) increase occurred. Data from electrophysiology experiments were analyzed using CLAMPFIT™ 9.0 (MOLECULAR DEVICES™) and MINI ANALYSIS™ (SYNAPTOSOFT™).

### Drug Administration

Clonazepam at indicated concentrations (0.0625 mg/kg˜0.5 mg/kg, SIGMA™) diluted in the vehicle solution (PBS with 0.5% methylcellulose) was administered by an intraperitoneal injection in a volume of 0.01 ml/kg 30 min before the behavioral tests. Oral administration is specifically contemplated for the methods described herein, and is well known for both benzodiazepines and non-BDZ GABA-A enhancers as described herein.

### Statistical Analysis

All data are shown as mean±s.e.m. and analyzed using Student's t-test, one-way ANOVA with Tukey's post hoc comparison, and two-way ANOVA with Bonferroni's post hoc comparison. All the statistical analyses were done using PRISM™ 4 (GRAPHPAD™)

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### REFERENCES

1. Wolff, M., Casse-Perrot, C.& Dravet, C. Severe myoclonic epilepsy of infancy (Dravet syndrome): natural history and neuropsychological findings. Epilepsia 47 (Suppl. 2), 45-48 (2006).
2. Genton, P., Velizarova, R. & Dravet, C. Dravet syndrome: the long-term outcome. Epilepsia 52 (Suppl 2), 44-49 (2011).
3. Li, B. M. et al. Autismin Dravet syndrome: prevalence, features, and relationship to the clinical characteristics of epilepsy and mental retardation. Epilepsy Behay. 21, 291-295 (2011).
4. Brunklaus, A., Donis, L. & Zuberi, S. M. Comorbidities and predictors of health related quality of life in Dravet syndrome. Epilepsia 52, 1476-1482 (2011).
5. Besag, F. M. Behavioral aspects of pediatric epilepsy syndromes. Epilepsy Behay. 5 (Suppl. 1), 3-13 (2004).
6. Mahoney, K. et al. Variable neurologic phenotype in a GEFS1 family with a novel mutation in SCN1A. Seizure 18, 492-497 (2009).
7. Claes, L. et al. De novo mutations in the sodium-channel gene SCN1Acause severe myoclonic epilepsy of infancy. Am. J. Hum. Genet. 68, 1327-1332 (2001).
8. Catterall, W. A. From ionic currents to molecular mechanisms: the structure and function of voltage-gated sodium channels. Neuron 26, 13-25 (2000).
9. Yu, F. H. et al. Reduced sodium current in GABAergic interneurons in a mouse model of severe myoclonic epilepsy in infancy. Nature Neurosci. 9, 1142-1149 (2006).
10. Kalume, F., Yu, F. H., Westenbroek, R. E., Scheuer, T. & Catterall, W. A. Reduced sodium current in Purkinje neurons from NaV1.1 mutant mice: implications for ataxia in severe myoclonic epilepsy in infancy. J. Neurosci. 27, 11065-11074 (2007).
11. Oakley, J. C., Kalume, F., Yu, F. H., Scheuer, T. & Catterall, W. A. Temperature- and age-dependent seizures in a mouse model of severe myoclonic epilepsy in infancy. Proc. Natl Acad. Sci. USA 106, 3994-3999 (2009).
12. Han, S. et al. NaV1.1 channels are critical for intercellular communication in the suprachiasmatic nucleus and for normal circadian rhythms. Proc. Natl Acad. Sci. USA 109, E368-E377 (2012).
13. Catterall, W. A., Kalume, F. & Oakley, J. C. NaV1.1 channels and epilepsy. J. Physiol. (Lond.) 588, 1849-1859 (2010).
14. Westenbroek, R. E., Merrick, D. K. & Catterall, W. A. Differential subcellular localization of the RI and RII Na1 channel subtypes in central neurons. Neuron 3, 695-704 (1989).
15. Van Wart, A., Trimmer, J. S. & Matthews, G. Polarized distribution of ion channels within microdomains of the axon initial segment. J. Comp. Neurol. 500, 339-352 (2007).
16. Ogiwara, I. et al. NaV1.1 localizes to axons of parvalbumin-positive inhibitory interneurons: a circuit basis for epileptic seizures in mice carrying an Scn1a gene mutation. J. Neurosci. 27, 5903-5914 (2007).
17. Cheah, C. S. et al. Specific deletion of NaV1.1 channels in inhibitory interneurons causes seizures and premature death in mouse model of dravet syndrome. Proc. Natl Acad. Sci. USA. (in the press).

18. Pescucci, C. et al. 2q24-q31 deletion: report of a case and review of the literature. Eur. J. Med. Genet. 50, 21-32 (2007).
19. Ramoz, N., Cai, G., Reichert, J. G., Silverman, J. M. & Buxbaum, J. D. An analysis of candidate autism loci on chromosome 2q24-q33: evidence for association to the STK39 gene. Am. J. Med. Genet. B 147B, 1152-1158 (2008).
20. Weiss, L. A. et al. Sodium channels SCN1A, SCN2A and SCN3A in familial autism. Mol. Psychiatry 8, 186-194 (2003).
21. O'Roak, B. J. et al. Exome sequencing in sporadic autism spectrum disorders identifies severe de novo mutations. Nature Genet. 43, 585-589 (2011).
22. O'Roak, B. J. et al. Sporadic autism exomes reveal a highly interconnected protein network of de novo mutations. Nature 485, 246-250 (2012).
23. Chao, H. T. et al. Dysfunction in GABA signalling mediates autism-like stereotypes and Rett syndrome phenotypes. Nature 468, 263-269 (2010).
24. Paluszkiewicz, S. M., Martin, B. S. & Huntsman, M. M. Fragile X syndrome: the GABAergic system and circuit dysfunction. Dev. Neurosci. 33, 349-364 (2011).
25. Pen~agarikano, O. et al. Absence of CNTNAP2 leads to epilepsy, neuronal migration abnormalities, and core autism-related deficits. Cell 147, 235-246 (2011).
26. Hussman, J. P. Suppressed GABAergic inhibition as a common factor in suspected etiologies of autism. J. Autism Dev. Disord. 31, 247-248 (2001).
27. Rubenstein, J. L. & Merzenich, M. M. Model of autism: increased ratio of excitation/inhibition in key neural systems. Genes Brain Behay. 2, 255-267 (2003).
28. Markram, K. & Markram, H. The intense world theory—a unifying theory of the neurobiology of autism. Front. Hum. Neurosci. 4, 224 (2010).
29. Yizhar, O. et al. Neocortical excitation/inhibition balance in information processing and social dysfunction. Nature 477, 171-178 (2011).
30. Moretti, P., Bouwknecht, J. A., Teague, R., Paylor, R.& Zoghbi, H. Y. Abnormalities of social interactions and home-cage behavior in a mouse model of Rett syndrome. Hum. Mol. Genet. 14, 205-220 (2005).
31. Geschwind, D. H. Genetics of autism spectrum disorders. Trends Cogn. Sci. 15, 409-416 (2011).
32. Stockhorst, U. & Pietrowsky, R. Olfactory perception, communication, and the nose-to-brain pathway. Physiol. Behay. 83, 3-11 (2004).
33. Wang, Z. et al. Pheromone detection in male mice depends on signaling through the type 3 adenylyl cyclase in the main olfactory epithelium. J. Neurosci. 26, 7375-7379 (2006).
34. Silverman, J. L., Yang, M., Lord, C. & Crawley, J. N. Behavioural phenotyping assays for mouse models of autism. Nature Rev. Neurosci. 11, 490-502 (2010).
35. Yang, M. et al. Low sociability in BTBR T 1 tf/J mice is independent of partner strain. Physiol. Behay. (8 Jan. 2012).

36. Gomot, M. et al. Change detection in children with autism: an auditory event related fMRI study. Neuroimage 29, 475-484 (2006).
37. Long, J. E., Cobos, I., Potter, G. B. & Rubenstein, J. L. Dlx1&2 and Mash1 transcription factors control MGE and CGE patterning and differentiation through parallel and overlapping pathways. Cereb. Cortex 19 (Suppl. 1), i96-i106 (2009).
38. Potter, G. B. et al. Generation of Cre-transgenic mice using Dlx1/Dlx2 enhancers and their characterization in GABAergic interneurons. Mol. Cell. Neurosci. 40, 167-186 (2009).
39. Rudolph, U. & Knoflach, F. Beyond classical benzodiazepines: novel therapeutic potential of GABA-A receptor subtypes. Nature Rev. Drug Discov. 10, 685-697 (2011).
40. Low, K. et al. Molecular and neuronal substrate for the selective attenuation of anxiety. Science 290, 131-134 (2000).
41. Abrahams, B. S.& Geschwind, D. H. Advances in autism genetics: on the threshold of a new neurobiology. Nature Rev. Genet. 9, 341-355 (2008).
42. Yakoub, M., Dulac, O., Jambaque, I., Chiron, C.& Plouin, P. Early diagnosis of severe myoclonic epilepsy in infancy. Brain Dev. 14, 299-303 (1992).
43. Rossignol, E. Genetics and function of neocortical GABAergic interneurons in neurodevelopmental disorders. Neural Plast. 2011, 649325 (2011).
44. Paluszkiewicz, S. M., Olmos-Serrano, J. L., Corbin, J. G. & Huntsman, M. M. Impaired inhibitory control of cortical synchronization in fragile X syndrome. J. Neurophysiol. 106, 2264-2272 (2011).
45. McKernan, R. M. et al. Sedative but not anxiolytic properties of benzodiazepines are mediated by the GABA(A) receptor alpha1 subtype. Nature neuroscience 3, 587-592, doi:10.1038/75761 (2000).
46. Rudolph, U. et al. Benzodiazepine actions mediated by specific gamma-aminobutyric acid(A) receptor subtypes. Nature 401, 796-800, doi:10.1038/44579 (1999).
47. Bateson, A. N. Basic pharmacologic mechanisms involved in benzodiazepine tolerance and withdrawal. Current pharmaceutical design 8, 5-21 (2002).
48. Atack, J. R. et al. TPA023 [7-(1,1-dimethylethyl)-6-(2-ethyl-2H-1,2,4-triazol-3-ylmethoxy)-3-(2-fluorophenyl)-1,2,4-triazolo[4,3-b]pyridazine], an agonist selective for alpha2- and alpha3-containing GABA-A receptors, is a nonsedating anxiolytic in rodents and primates. The Journal of pharmacology and experimental therapeutics 316, 410-422, doi:10.1124/jpet.105.089920 (2006).
49. Rudolph, U. & Mohler, H. Analysis of GABA-A receptor function and dissection of the pharmacology of benzodiazepines and general anesthetics through mouse genetics. Annual review of pharmacology and toxicology 44, 475-498, doi:10.1146/annurev.pharmtox.44.101802.121429 (2004).
50. Crestani, F. et al. Trace fear conditioning involves hippocampal alpha5 GABA(A) receptors. Proc Natl Acad Sci USA 99, 8980-8985, doi:10.1073/pnas.142288699 (2002).
51. Rudolph, U. & Mohler, H. GABA-based therapeutic approaches: GABA-A receptor subtype functions. Current opinion in pharmacology 6, 18-23, doi:10.1016/j.coph.2005.10.003 (2006).
52. van Rijnsoever, C. et al. Requirement of alpha5-GABA-A receptors for the development of tolerance to the sedative action of diazepam in mice. J Neurosci 24, 6785-6790, doi:10.1523/JNEUROSCI.1067-04.2004 (2004).
53. McKernan R M, Rosahl T W, Reynolds D S, et al. (2000). “Sedative but not anxiolytic properties of benzodiazepines are mediated by the GABA(A) receptor alpha1 subtype”. Nat. Neurosci. 3 (6): 587-92. doi:10.1038 PMID 10816315.
54. Atack J R (2005). “The benzodiazepine binding site of GABA(A) receptors as a target for the development of novel anxiolytics” http://www.expertopin.com/doi/abs/10.1517/13543784.14.5.601). Expert Opin Investig Drugs 14 (5): 601-18. doi:10.1517/13543784.14.5.601 http://dx.doi.org/10.1517%2F13543784.14.5.601). PMID 15926867//www.ncbi.nlm.nih.gov/pubmed/15926867). 13543784.14.5.601.
55. Morris H V, Dawson G R, Reynolds D S, Atack J R, Stephens D N (2006). “Both alpha2 and alpha3 GABA-A receptor subtypes mediate the anxiolytic properties of benzodiazepine site ligands in the conditioned emotional response paradigm” Eur. J. Neurosci. 23 (9): 2495-504. doi:10.1111/j.1460-9568.2006.04775.x. PMID 16706856.
56. Mathiasen L S, Rodgers R J, Mirza N R (2007). “Comparative effects of nonselective and subtype-selective gamma-aminobutyric acid A receptor positive modulators in the rat-conditioned emotional response test “Behav Pharmacol 18 (3): 191-203. doi:10.1097/FBP.0b013e32814fcdd4. PMID 17426483
57. Ujfalussy B, Kiss T, Orbán G, Hoffmann W E, Erdi P, Hajós M (2007). “Pharmacological and computational analysis of alpha-subunit preferential GABA(A) positive allosteric modulators on the rat septo-hippocampal activity” Neuropharmacology 52 (3): 733-43. doi:10.1016/j.neuropharm.2006.09.022 PMID 17113111
58. Mirza N R, Rodgers R J, Mathiasen L S (2006). “Comparative cue generalization profiles of L-838, 417, SL651498, zolpidem, CL218,872, ocinaplon, bretazenil, zopiclone, and various benzodiazepines in chlordiazepoxide and zolpidem drug discrimination” J. Pharmacol. Exp. Ther. 316 (3): 1291-9. doi:10.1124/jpet.105.094003. PMID 16339395
59. Mathiasen L, Mirza N R (2005). “A comparison of chlordiazepoxide, bretazenil, L838,417 and zolpidem in a validated mouse Vogel conflict test”. Psychopharmacology (Berl.) 182 (4): 475-84. doi:10.1007/s00213-005-0119-z ISBN [[Special:BookSources/2-13-005011-9|2-13-005011-9]]. PMID 16133136
60. Carling R W, Madin A, Guiblin A, et al. (2005). “7-(1,1-Dimethylethyl)-6-(2-ethyl-2H-1,2,4-triazol-3-ylmethoxy)-3-(2-fluorophenyl)-1,2,4-triazolo[4,3-b]pyridazine: a functionally selective gamma-aminobutyric acid(A) (GABA(A)) alpha2/alpha3-subtype selective agonist that exhibits potent anxiolytic activity but is not sedating in animal models”. J. Med. Chem. 48 (23): 7089-92. doi:10.1021/jm058034a PMID 16279764.
61. Han, S. et al. Autistic-like behaviour in Scn1a^{+/-} mice and rescue by enhanced GABA-mediated neurotransmission. Nature 489, 385-390, doi:10.1038/nature11356 (2012).
62. Gordon, D. et al. Tissue-specific expression of the RI and RII sodium channel subtypes. Proc Natl Acad Sci USA 84, 8682-8686 (1987).
63. Deacon, R. M. Assessing nest building in mice. Nat Protoc 1, 1117-1119 (2006).
64. Tabuchi, K. et al. A neuroligin-3 mutation implicated in autism increases inhibitory synaptic transmission in mice. Science 318, 71-76 (2007).
65. Yang, M. et al. Low sociability in BTBR T+tf/J mice is independent of partner strain. Physiol Behav (2012).
66. Eckel-Mahan, K. L. et al. Circadian oscillation of hippocampal MAPK activity and cAmp: implications for memory persistence. Nat Neurosci 11, 1074-1082 (2008).
67. Villeda, S. A. et al. The ageing systemic milieu negatively regulates neurogenesis and cognitive function. Nature 477, 90-94 (2011).
68. Arruda-Carvalho, M., Sakaguchi, M., Akers, K. G., Josselyn, S. A. & Frankland, P. W. Posttraining ablation of adult-generated neurons degrades previously acquired memories. J Neurosci 31, 15113-15127 (2011).
69. Shamir, A. et al. The importance of the NRG-1/ErbB4 pathway for synaptic plasticity and behaviors associated with psychiatric disorders. J Neurosci 32, 2988-2997 (2012).
70. Tai, C., Kuzmiski, J. B. & MacVicar, B. A. Muscarinic enhancement of R-type calcium currents in hippocampal CA1 pyramidal neurons. J Neurosci 26, 6249-6258 (2006).
71. McFarlane, H. G. et al. Autism-like behavioral phenotypes in BTBR T+tf/J mice. Genes, brain, and behavior 7, 152-163, doi:10.1111/j.1601-183X.2007.00330.x (2008).

### Example 4

### Enhancement of Inhibitory Neurotransmission by GABA-A Receptors Having α2,3-Subunits Ameliorates Behavioral Deficits in a Mouse Model of Autism

### Reduced Inhibitory Neurotransmission in BTBR Mice

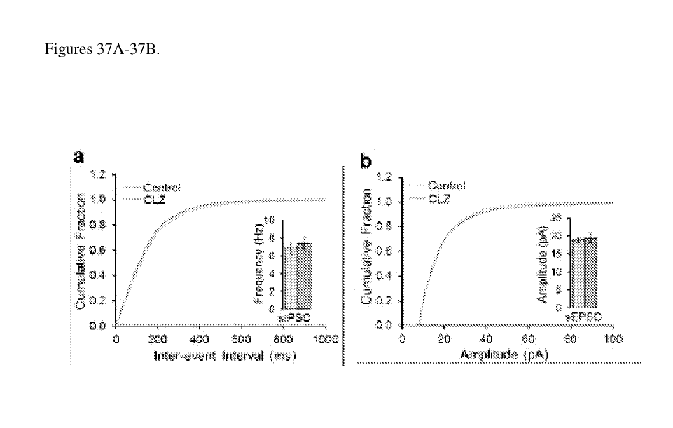

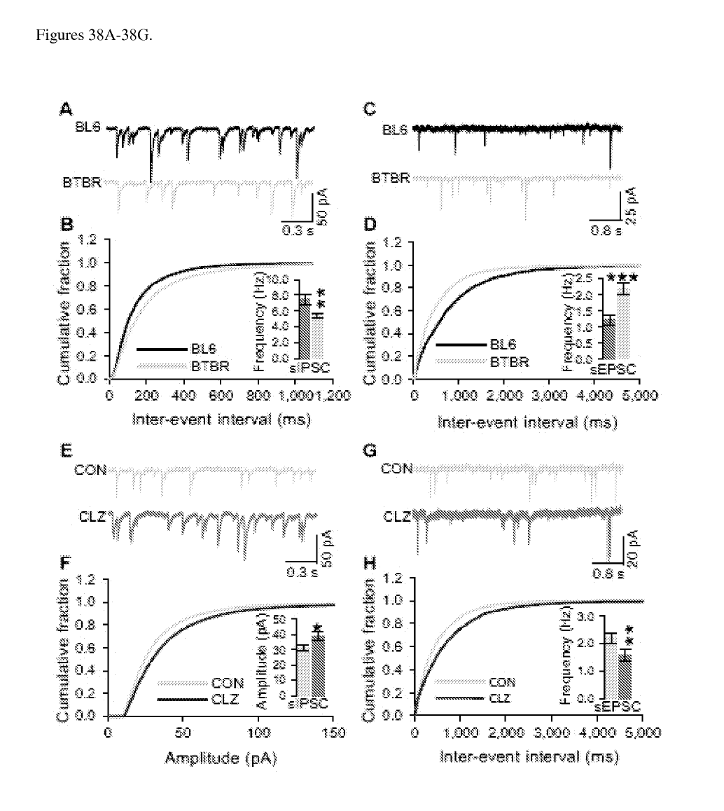

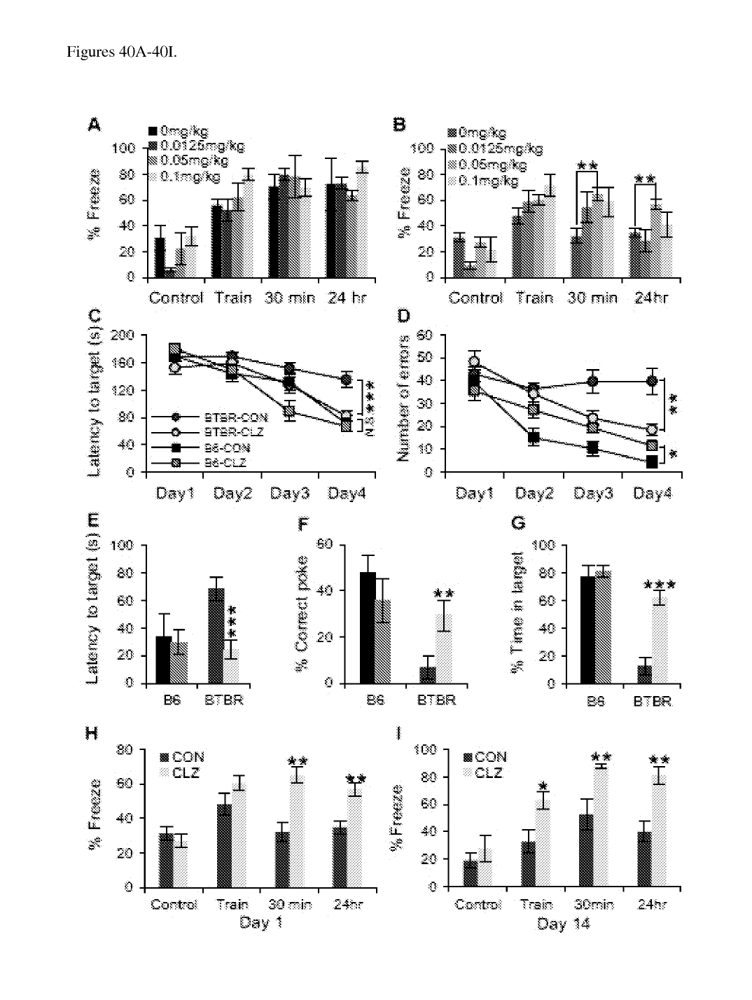

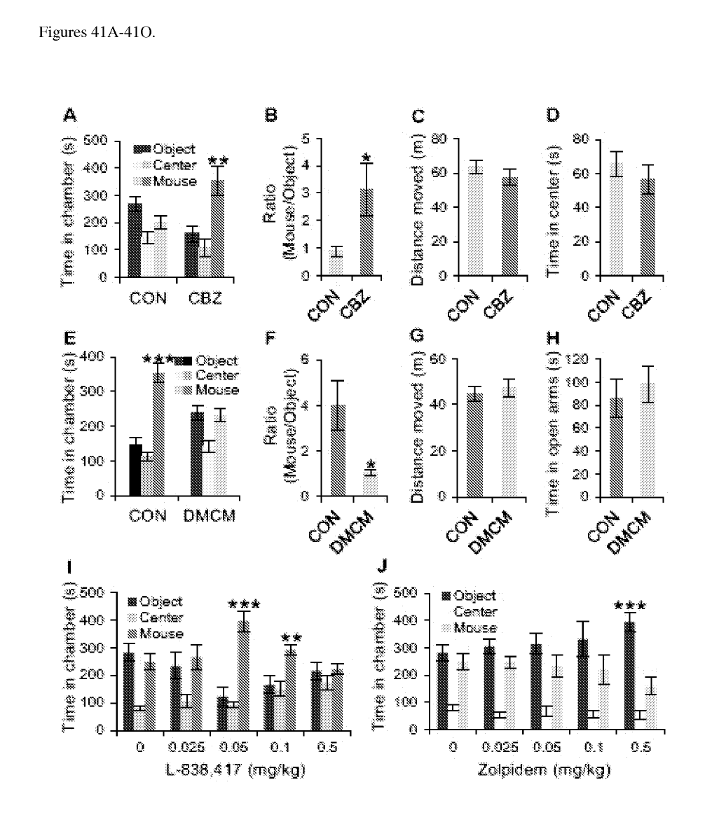

A challenge for research on BTBR mice is selection of an appropriate control mouse line for comparison, as different inbred strains differ in various behavioral and cognitive measures. Consistent with previous work, the inventors chose to focus their study on differences in neurotransmission, behavior, and cognition between BTBR and C57BL/6J mice (see Supplementary Information for more discussion and references). To test the hypothesis that BTBR mice may have reduced inhibitory neurotransmission, the inventors measured spontaneous excitatory and inhibitory postsynaptic currents in the CA1 region of hippocampal slices from age-matched (P21-25) BTBR and C57BL/6J mice. Although the amplitude of spontaneous inhibitory postsynaptic current (IPSC) was not altered in BTBR hippocampal slices compared to the C57BL/6J hippocampal slices (FIG. 42A), the frequency of spontaneous IPSC was significantly reduced in BTBR hippocampal slices when compared with the C57BL/6J hippocampal slices (FIGS. 38A, 38B). In conjunction with decreased inhibitory neurotransmission, the amplitude and the frequency of spontaneous excitatory post-synaptic current (EPSC) were substantially increased in BTBR hippocampal slices when compared with C57BL/6J hippocampal slices (FIGS. 38C, 38D, and 42B). In control recordings of miniature postsynaptic currents, in which action potentials were blocked with tetrodotoxin (TTX), the amplitude and frequency of miniature IPSC and the frequency of miniature EPSC were unaltered (FIGS. 42E-42G). However, the amplitude of miniature EPSCs was significantly increased in BTBR hippocampal slices when compared with 57BL/6J hippocampal slices (FIG. 42H). Surprisingly, these studies reveal that BTBR mice have a deficit in inhibitory neurotransmission compared to the control strain C57BL/6J, which is caused by reduced frequency of inhibitory synaptic events without a corresponding decrease in postsynaptic response. This deficit in inhibitory neurotransmission is accompanied by a corresponding increase in excitatory neurotransmission. These results indicate that constitutively decreased inhibitory neurotransmission may be a contributing factor to the autistic-like behaviors in BTBR mice.

### Increased GABAergic Inhibitory Neurotransmission in Response to Benzodiazepines

Attempts to reverse autistic-like traits by rebalancing the ratio of excitatory to inhibitory neurotransmission through pharmacological treatments that reduce excitatory neurotransmission have met with only partial success because of their limited efficacy and unwanted side effects in control groups (Berry-Kravis et al., 2012; Gandal et al., 2012; Henderson et al., 2012; Michalon et al., 2012; Yang et al., 2012). The results of FIGS. 38A-38D indicate that enhancing inhibitory neurotransmission will likely be effective. The GABA-A receptor is a heteropentameric ligand-gated chloride channel that mediates the major inhibitory effects of GABAergic neurotransmission in the brain. Subsynaptic GABA-A receptor subtypes are composed of two α, two β, and one γ subunit (Fritschy and Mohler, 1995). The action of GABA at these ionotropic receptors is increased through positive allosteric modulation by benzodiazepines, which are used to treat anxiety, insomnia, and epilepsy (Rudolph and Knoflach, 2011). In order to determine whether treatment with a benzodiazepine reverses the constitutively decreased GABAergic inhibitory signaling, the inventors treated C57BL/6J and BTBR hippocampal slices with 0.5 μM clonazepam, a broad-acting, traditional benzodiazepine. These recordings revealed increased spontaneous IPSC amplitude (FIGS. 38E, 38F) and frequency (FIG. 42C) in BTBR slices. In contrast, a significant increase of spontaneous IPSC amplitude (FIG. 42I) but no change in IPSC frequency (FIG. 42J) was observed in C57BL/6J slices. The increased GABAergic signaling after treatment with clonazepam led to a decrease in frequency of spontaneous EPSCs (FIGS. 38G, 38H), without change in amplitude in BTBR hippocampal slices (FIG. 42D). Interestingly, the frequency of spontaneous EPSC was also decreased by clonazepam (FIG. 42K), without change in amplitude (FIG. 42L) in C57BL/6J slices. These data indicate that low-dose clonazepam can reverse the underlying deficit in spontaneous GABAergic inhibitory neurotransmission in BTBR mice.

Improvement of Social Interaction by Treatment with Clonazepam

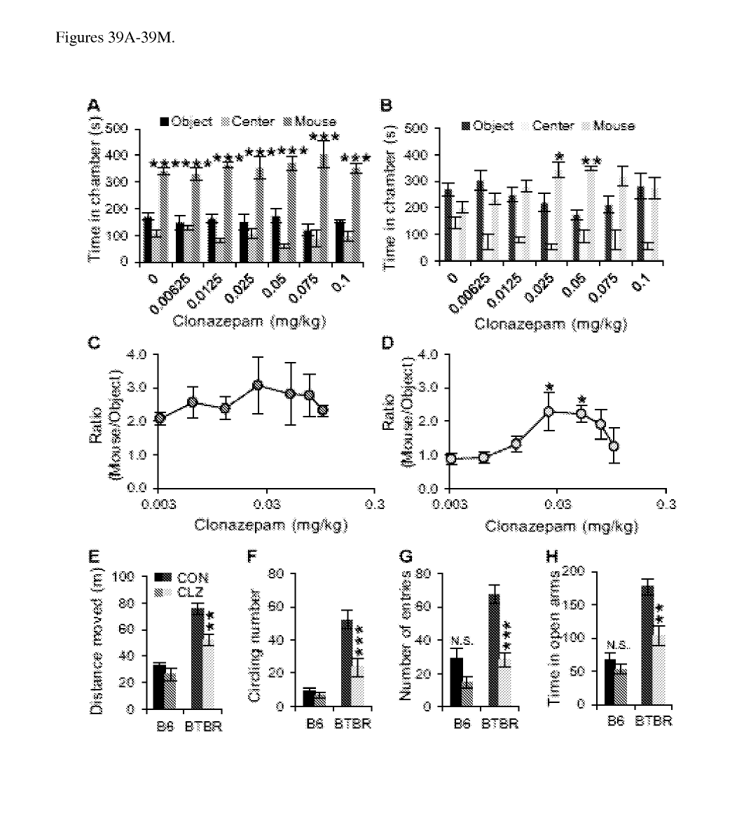

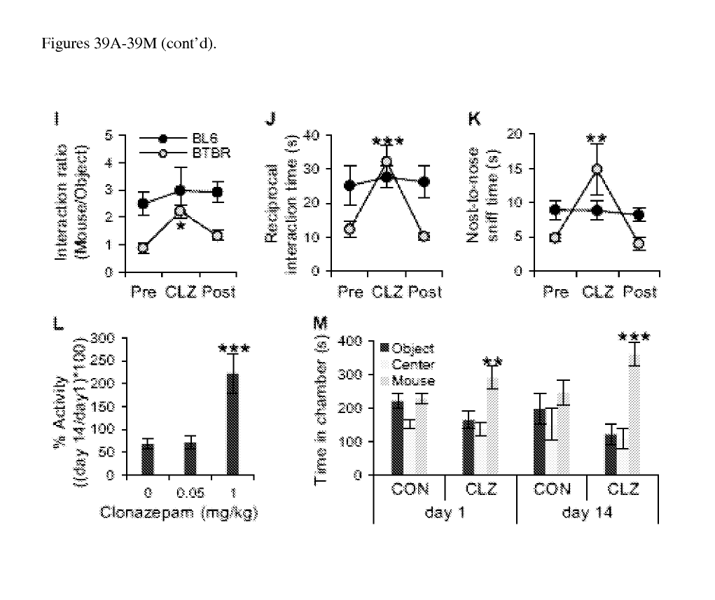

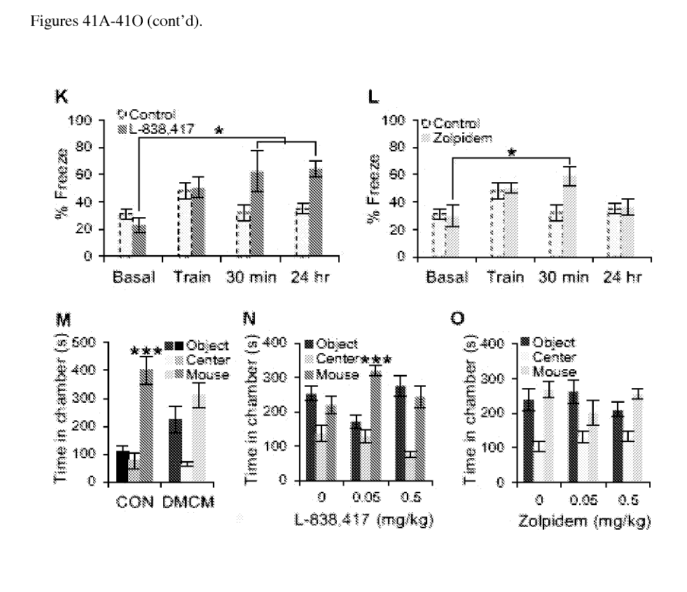

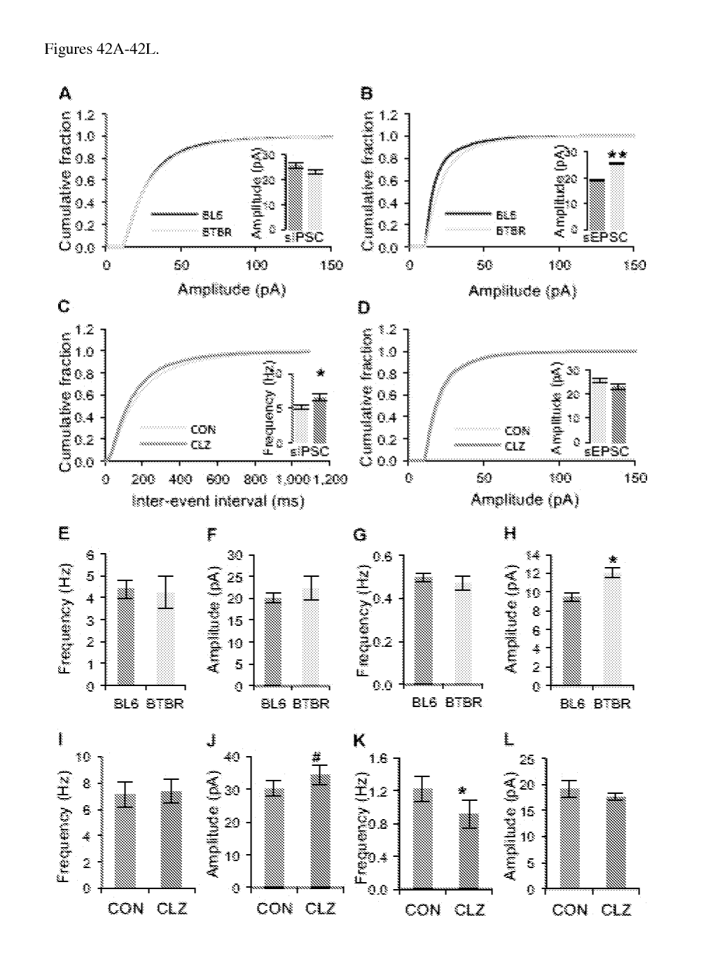

To test the behavioral effects of enhancing inhibitory neurotransmission in BTBR mice, the inventors injected low non-sedating/non-anxiolytic doses of clonazepam intraperitoneally 30 min prior to behavioral tests. In the three-chamber social interaction test, acute clonazepam treatment had no effect on social interactions of C57BL/6J mice (FIGS. 39A, 43A) but increased social interactions in BTBR, with a maximal effect at 0.05 mg/kg (FIGS. 39B, 43B) and no sedation (FIG. 43H). Measurements of the time of interaction of the test mouse with a stranger mouse vs. a novel object during three-chamber tests showed that the C57BL/6J mice are unaffected by any of the test doses (FIG. 39C), whereas improvement of the social deficit in BTBR mice by clonazepam is strikingly dose-dependent (FIG. 39D). Interestingly, the improved social interactions in BTBR mice was lost at higher doses of clonazepam (FIGS. 39B, 39D). Other behaviors in BTBR mice were also rescued by low-dose clonazepam. In the open field test, a single injection of 0.05 mg/kg clonazepam significantly reduced hyperactivity, measured as the total distance moved (FIG. 39E), and stereotyped circling behavior, measured as the number of 360° rotations (FIG. 39F). In contrast, these behaviors in C57BL/6J mice were unaffected by low-dose clonazepam. These low doses of clonazepam had little effect on anxiety-like behaviors of C57BL/6J mice, such as avoidance of the center of an open field or the open arms of an elevated plus maze (FIG. 39G, 39H). However, compared to C57BL/6J, BTBR mice visited the center in the open field significantly more frequently and spent more time in open arms during the elevated-plus maze test under control conditions, as if they were less anxious than C57BL/6J mice, and these indicators of abnormally low anxiety in BTBR mice were changed toward the values for C57BL/6J mice after treatment with 0.05 mg/kg clonazepam (FIG. 39G, 39H) without sedation (FIG. 43I).

Three sets of identical three-chamber tests at one-week intervals with the same group of mice showed the reversibility of rescue of social interaction deficits in BTBR by 0.05 mg/kg clonazepam (FIGS. 39I-39K and 42C-42F). In the first trial, with no treatment, BTBR mice displayed characteristic social interaction deficits when compared with C57BL/6J mice for interaction ratio with a stranger mouse vs. an inanimate object (FIG. 39I). In a second identical trial one week later, these social deficits were improved by treatment with 0.05 mg/kg clonazepam 30 min before testing in the same group of BTBR mice (FIG. 39I).

In the third trial one week later, injection of vehicle had no effect to rescue the social behavior in the same mice (FIG. 39I). None of these treatments had any significant effect on C57BL/6J mice (FIG. 39I). This reversible effect of clonazepam treatment was also observed in the open-field reciprocal social interaction test in an intra-group comparison setting in which the test mouse cannot escape from the social stimulus provided by the stranger mouse. During three identical sets of reciprocal social interaction tests, impaired reciprocal social interaction, and nose-to-nose contact in BTBR mice were significantly enhanced in the 0.05 mg/kg clonazepam treated group, whereas C57BL/6J mice were unaffected (FIGS. 39J and 39K). These data show that low-dose clonazepam increases social interaction in BTBR mice within a narrow effective dose range.

Long-term treatment with the standard high doses of benzodiazepines causes tolerance in humans (Bateson, 2002). Tolerance to the sedative effects of high-dose clonazepam begins on Day 7 and reaches maximum on Day 14 in mice (Galpern et al., 1991; Loscher et al., 1996). To test tolerance in this context in BTBR mice, 0.05 mg/kg clonazepam was injected intraperitoneally daily for 14 days. For untreated animals, the locomotor activity in an open field was 68±11% of normal on Day 14 compared to Day 1 (FIG. 39L), likely because the open field chamber is familiar from their experience on Day 1 and they do not explore it as extensively on Day 14. Treatment with 0.05 mg/kg clonazepam did not have any effect on locomotor activity and did not alter the ratio of activity on Days 1 and 14 (FIG. 39L). In contrast, injection of 1 mg/kg clonazepam for 14 days caused significant tolerance, as indicated by the large increase in level of locomotor activity on Day 14 compared to Day 1 due to the repeated administration of the drug (222±42%, FIG. 39L). In the three-chamber social interaction test, 0.05 mg/kg clonazepam significantly increased social interactions on Day 1, and this effect was fully retained and even increased after 14 days of repeated treatment (FIG. 2M). These data indicate that repeated treatment with 0.05 mg/kg dose of clonazepam does not elicit tolerance to its rescue of social interaction behavior in the time frame of development of tolerance for the sedative effects of the drug.

Amelioration of Cognitive Deficits by Treatment with Clonazepam

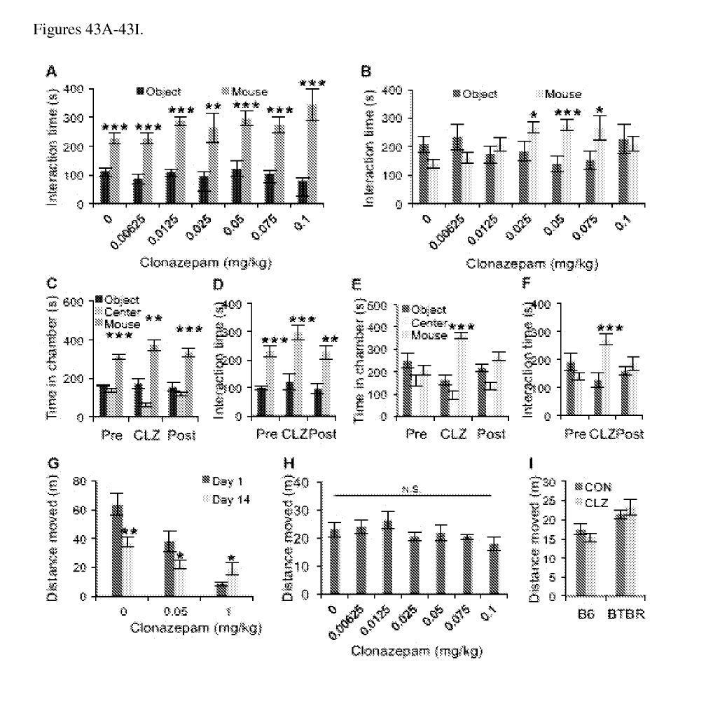

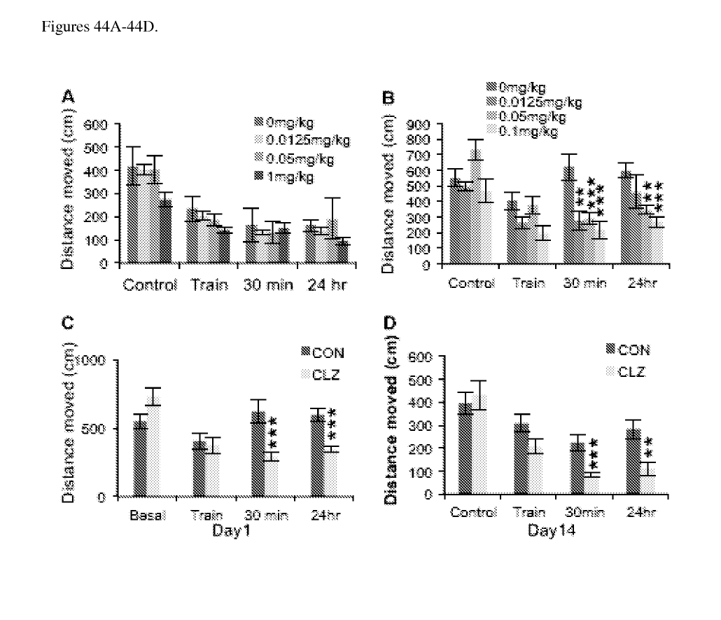

Cognitive problems are often associated with ASD (Zoghbi and Bear, 2012), and BTBR mice are known to have impaired fear memory (MacPherson et al., 2008). To test the effects of low-dose clonazepam on cognitive deficits, the inventors performed context-dependent fear conditioning after treatment with increasing doses of clonazepam in both BTBR and C57BL/6J mice (FIGS. 40A, 40B). Short-term (30 min) and long-term (24 h) memory performance in fear conditioning to the spatial context in BTBR mice were improved by treatment with 0.05 mg/kg clonazepam, but no significant effects were observed after treatment with 0.0125 mg/kg or 0.1 mg/kg clonazepam (FIGS. 40B, 44B). In contrast, no cognitive changes were observed in C57BL/6J mice at any dose (FIGS. 40A, 44A). To test spatial learning and memory in the absence of fear, the inventors performed the Barnes circular maze test in which mice rapidly escape a brightly lit field by learning the location of a hole with a dark refuge at its periphery. BTBR mice failed to improve their performance during repeated training sessions, and this learning impairment was improved by clonazepam treatment (FIGS. 40C, 40D). In probe trials, in which mice search for a learned refuge that has been removed, spatial memory in BTBR mice was also increased by clonazepam treatment (FIG. 40E-40G). In contrast, C57BL/6J mice displayed improved learning performance during repeated training sessions (FIGS. 40C and 40D), and normal spatial memory during the probe trial (FIG. 40E-40G), regardless of clonazepam treatment. These data indicate that spatial learning in BTBR mice is substantially restored by treatment with low-dose clonazepam.

To determine whether tolerance develops to clonazepam the inventors performed context-dependent fear conditioning on Day 1 of clonazepam injection and Day 14 of daily treatment with 0.05 mg/kg clonazepam. Regardless of the treatment period, the context-dependent fear memory improved by clonazepam treatment in BTBR mice (FIGS. 40H, 40I, 44C, 44D), indicating that low-dose clonazepam treatment does not cause tolerance to its effects on cognitive deficit in BTBR mice.

### Opposing Effects of Positive and Negative Allosteric Modulators of GABA-A Receptors

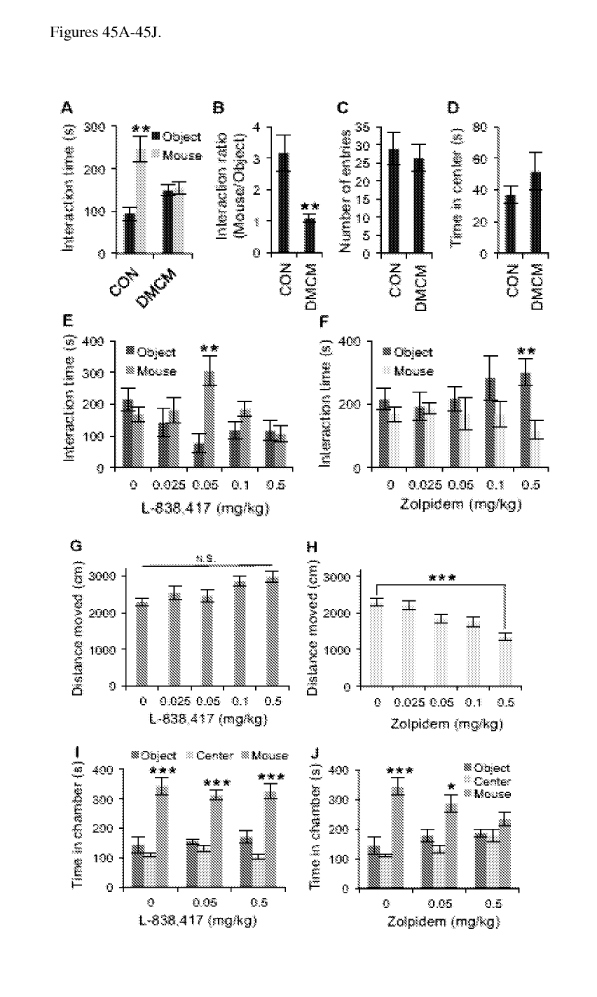

In order to test the effect of other classes of GABA-A receptor modulators on social behavior, the inventors treated BTBR mice with low-dose clobazam, an atypical benzodiazepine that is a positive allosteric modulator of GABA-A receptors (Farrell, 1986). A single injection of 0.05 mg/kg clobazam 30 min before the three-chamber ameliorated social interaction deficits in BTBR mice (FIGS. 41A, 41B) without sedative (FIG. 41C) or anxiolytic effects (FIG. 41D). To further test the idea that the E/I balance is critical for normal social behaviors, the inventors treated C57BL/6J and 129SvJ WT mice with low-dose DMCM, a negative allosteric modulator of the GABA-A receptor function as assessed from IPSPs recorded in hippocampalslices and from behavioral tests (Rovira and Ben-Ari, 1993; Savic et al., 2006). In the three-chamber test, a single injection of a low non-convulsant/non-anxiogenic dose of DMCM (0.2 mg/kg) (Savic et al., 2004) 30 min prior to the test substantially reduced normal social interaction behavior in both C57BL/6J and 129SvJ mice (FIGS. 41E, 41F, 41M, and 45A). In contrast, the general locomotor behavior and anxiety levels were not altered by 0.2 mg/kg DMCM (FIGS. 41G, 41H).

The results with DMCM support the notion that impairment of GABAergic neurotransmission might contribute to autistic-like behaviors.

### Rescue by α2, α3-Specific Positive Allosteric Modulators of GABA-A Receptors

Diversity of GABA receptor function is conferred by more than 20 different subunits, and receptors with different a subunits play distinct roles in the physiological and pharmacological actions of GABA and benzodiazepines (Fritschy and Mohler, 1995; Harmar et al., 2009; Rudolph and Knoflach, 2011; Rudolph and Mohler, 2004; Smith and Olsen, 1995). The inventors tested the effects of subunit-selective positive allosteric modulators of GABA-A receptors on social behavior in BTBR mice and C57BL/6J mice. A low dose of the α2,3 subunit-selective positive allosteric modulator L-838,417 (Low et al., 2000; Mathiasen et al., 2008) increased social interactions in BTBR mice, with maximal effective dose of 0.05 mg/kg, and the beneficial effect was lost when the dose increased (FIGS. 41I and 45E). In contrast, L-838,417 did not change the social interaction behavior of C57BL/6J mice (FIG. 45I). Moreover, the α1 subunit-selective positive GABA-A modulator zolpidem (Mathiasen et al., 2008; Sieghart, 1995) failed to show beneficial effects in BTBR mice and actually aggravated their social interaction deficit at high doses (FIGS. 41J and 45F). Interestingly, a high dose of zolpidem also impaired social behavior in C57BL/6J mice (FIG. 45J).

Total movement tended to increase at high doses of L-838,417 (FIG. 45G; not significant), but significantly decreased at 0.5 mg/kg zolpidem (FIG. 45H). These results indicate that different subtypes of GABA-A receptors may have opposite roles in social behavior, with activation of GABA-A receptors containing α2,3 subunits favoring and of GABA-A receptors with α1 subunits reducing social interaction, respectively.

Subunit-selective GABA-A receptor modulators may also have an important effect on cognitive behaviors. In the context-dependent fear conditioning test, treatment with 0.05 mg/kg L-838,417 improved short-term (30 min) and long-term (24 h) spatial memory in BTBR mice (FIG. 41K), whereas 0.05 mg/kg zolpidem enhanced short-term memory, but not long-term memory (FIG. 41L). These datashow that α2,3 subunit-containing GABA-A receptors may also be important for cognitive behaviors in BTBR mice. The bell-shaped dose-response curves observed for both L-838,417 and clonazepam may explain why high-dose benzodiazepine treatment for prevention of anxiety and seizures has not been reported to improve autistic traits in ASD patients. As illustrated in FIGS. 41N and 41O, treatment with low doses of L-838,417 also improves social interactions in the Scn1a+/− mice, a model of Dravet Syndrome with severe autistic-like behaviors (Han et al., 2012), within a narrow dose range. In contrast, similar treatment with zolpidem is not effective. Altogether, these experiments show that treatment with an α2,3-selective positive allosteric modulator of GABA-A receptors is sufficient to rescue autistic-like behaviors and cognitive deficit in both a monogenic model of autism-spectrum disorder and the BTBR mouse model of idiopathic autism.

The inventors' results on mouse models of autism support the hypothesis that social and cognitive in ASDs is caused by an increased ratio of excitatory to inhibitory synaptic transmission (Gatto and Broadie, 2010; Han et al., 2012; Markram and Markram, 2010; Rubenstein and Merzenich, 2003). The inventors found that autistic BTBR mice have constitutively reduced inhibitory neurotransmission in the hippocampus and that enhancement of their inhibitory neurotransmission with positive allosteric modulators of GABA-A receptors improved autism-related traits. Conversely, it was found that global pharmacological reduction of inhibitory neurotransmission by the negative allosteric modulator DMCM was sufficient to induce some autism-related behaviors in C57BL/6J and 129SvJ WT mice. These results are most consistent with the hypotheses that reduced inhibitory neurotransmission is sufficient to induce autistic-like behaviors in mice and that enhanced inhibitory neurotransmission can reverse autistic-like behaviors. However, even though the BTBR mouse has been widely used as an animal model of autism, it is not yet fully understood which genetic changes lead to its autistic-like behaviors (Jones-Davis et al., 2013) or whether similar genetic changes are among the large number of DNA polymorphisms that have been implicated in human autism. Similarly, even though treatment of C57BL/6J mice and 129SvJ mice with a negativeallosteric modulator of GABA receptors, DMCM, induces specific autism-related behavioral impairments in these two mouse strains, it is not known whether this would be true for all mouse strains or whether decreasing the effectiveness of GABAergic inhibitory neurotransmission with DMCM or a related agent would cause any of the behavioral features of human autism.

GABA-A receptors with different subunit composition have different roles in synaptic transmission in hippocampal pyramidal neurons. Receptors containing α1 subunits mediate fast synaptic transmission at the synapses on distal dendrites, whereas receptors containing α2 subunits mediate fast synaptic transmission at synapses on the soma (Prenosil et al., 2006). Different inputs impinge on CA1 pyramidal neurons at these sites, providing a potential mechanism for understanding how specific modulation of these two receptor types with L-838,417 and zolpidem leads to differential effects on spatial learning and sedation. Because GABA-A receptors containing α2 subunits have specific physiological roles (Prenosil et al., 2006) and drug actions on them do not induce tolerance (Vinkers et al., 2012), they provide an attractive molecular target for therapy of autism and other disorders with reduced GABAergic inhibitory neurotransmission.

Therapeutic approaches to treat autistic traits in animal studies or in clinical trials have primarily focused on reducing excitatory neurotransmission in glutamatergic synapses to rebalance E/I ratio in autistic brain (Michalon et al., 2012; Yang et al., 2012). However, autistic-like behaviors in ASD mouse models are only partially reversed by drugs that inhibit excitatory neurotransmission, and these drugs also have unwanted side effects on wild-type mice (Henderson et al., 2012; Michalon et al., 2012; Yang et al., 2012). To overcome these drawbacks, the inventors focused on the opposing side, the GABAergic inhibitory transmission in autistic brain. Their results highlight the potential for therapy of autistic-like behaviors and cognitive deficit in ASD by low-dose treatment with subunit-selective benzodiazepines and other positive allosteric modulators of GABA-A receptors. At low doses that do not induce sedative or anxiolytic effects, the inventors found that clonazepam, clobazam, and L-838,417 all improved autistic-like behaviors and cognitive deficit in BTBR mice, supporting the hypothesis that α2,3 subunit-selective up-regulation of GABAergic neurotransmission is an effective treatment for these core features of autism. Consistent with this conclusion, the α1 subunit-selective positive allosteric modulator zolpidem had opposite effects. It is possible that the biphasic dose-response relationship of the positive allosteric modulators reflects their actions on receptors containing α2,3 subunits at low doses and on receptors containing α1 subunits at higher doses.

Although tolerance develops during prolonged treatment of patients with high doses of traditional benzodiazepines, these experiments indicate that tolerance is not induced by treatment of mice with low doses of clonazepam for 14 days, and α2,3-selective positive allosteric modulators of GABA-A receptors do not induce tolerance in rodents (Vinkers et al., 2012). Because of their broad availability and safety, benzodiazepines and other positive allosteric modulators of GABA-A receptors administered at low non-sedating, non-anxiolytic doses that do not induce tolerance deserve consideration as a near-term strategy to improve the core social interaction deficits and repetitive behaviors in ASD. Consistent with this view, Astra-Zeneca and the National Institutes of Health have initiated clinical trials of the α2,3-selective positive allosteric modulator of GABA-A receptors, AZD7325, for efficacy in autism (Clinical Trial Ref. No. NCT01966679).

### Experimental Procedures Summary

Adult male mice 6-10 months old were used for all behavioral tests. All mice were singly housed at least 1 week before the behavioral tests. All experiments with animals were performed according to the National Institutes of Health Guide for Care and Use of Laboratory Animals and were approved by the University of Washington Institutional Animal Care and Use Committee. The Open-field Test, Elevated Plus Maze Test, Three-chamber Test, Reciprocal Interaction Test, Barnes Circular Maze Test, and Contextual Fear Conditioning Test were carried out as described previously (Han et al., 2012) and in Example 3. As required for stable recordings of spontaneous synaptic activity, brain slices from 3-4 week-old mice were used for electrophysiological studies, which were carried out as described previously (Han et al., 2012). Drugs were administered and data were analyzed as described previously (Han et al., 2012) and herein in Example 3. All data are shown as mean±s.e.m. and analyzed using Student's t-test, one-way ANOVA with Tukey's post hoc comparison, and two-way ANOVA with Bonferroni's post hoc comparison. All the statistical analyses were done using Prism 6 (GraphPad).

### REFERENCES

Abrahams, B. S., and Geschwind, D. H. (2008). Advances in autism genetics: on the threshold of a new neurobiology. Nat Rev Genet 9, 341-355.
Auerbach, B. D., Osterweil, E. K., and Bear, M. F. (2011). Mutations causing syndromic autism define an axis of synaptic pathophysiology. Nature 480, 63-68.
Bateson, A. N. (2002). Basic pharmacologic mechanisms involved in benzodiazepine tolerance and withdrawal. Curr Pharm Des 8, 5-21.
Berry-Kravis, E. M., Hessl, D., Rathmell, B., Zarevics, P., Cherubini, M., Walton-Bowen, K., Mu, Y., Nguyen, D. V., Gonzalez-Heydrich, J., Wang, P. P., et al. (2012). Effects of STX209 (arbaclofen) on neurobehavioral function in children and adults with fragile X syndrome: a randomized, controlled, phase 2 trial. Sci Transl Med 4, 152ra127.
Chao, H. T., Chen, H., Samaco, R. C., Xue, M., Chahrour, M., Yoo, J., Neul, J. L., Gong, S., Lu, H. C., Heintz, N., et al. (2010). Dysfunction in GABA signalling mediates autism-like stereotypies and Rett syndrome phenotypes. Nature 468, 263-269.
Defensor, E. B., Pearson, B. L., Pobbe, R. L., Bolivar, V. J., Blanchard, D. C., and Blanchard, R. J. (2011). A novel social proximity test suggests patterns of social avoidance and gaze aversion-like behavior in BTBR T+tf/J mice. Behav Brain Res 217, 302-308.
Farrell, K. (1986). Benzodiazepines in the treatment of children with epilepsy. Epilepsia 27 Suppl 1, S45-52.
Fritschy, J. M., and Mohler, H. (1995). GABA-A-receptor heterogeneity in the adult rat brain: differential regional and cellular distribution of seven major subunits. J Comp Neurol 359, 154-194.
Galpern, W. R., Lumpkin, M., Greenblatt, D. J., Shader, R. I., and Miller, L. G. (1991). Chronic benzodiazepine administration. VII. Behavioral tolerance and withdrawal and receptor alterations associated with clonazepam administration. Psychopharmacology (Berl) 104, 225-230.
Gandal, M. J., Sisti, J., Klook, K., Ortinski, P. I., Leitman, V., Liang, Y., Thieu, T., Anderson, R., Pierce, R. C., Jonak, G., et al. (2012). GABAB-mediated rescue of altered excitatory-inhibitory balance, gamma synchrony and behavioral deficits following constitutive NMDAR-hypofunction. Transl Psychiatry 2, e142.
Gatto, C. L., and Broadie, K. (2010). Genetic controls balancing excitatory and inhibitory synaptogenesis in neurodevelopmental disorder models. Front Synaptic Neurosci 2, 4.
Geschwind, D. H. (2011). Genetics of autism spectrum disorders. Trends Cogn Sci 15, 409-416.
Han, S., Tai, C., Westenbroek, R. E., Yu, F. H., Cheah, C. S., Potter, G. B., Rubenstein, J. L., Scheuer, T., de la Iglesia, H. O., and Catterall, W. A. (2012). Autistic-like behaviour in Scn1a+/− mice and rescue by enhanced GABA-mediated neurotransmission. Nature 489, 385-390.
Harmar, A. J., Hills, R. A., Rosser, E. M., Jones, M., Buneman, O. P., Dunbar, D. R., Greenhill, S. D., Hale, V. A., Sharman, J. L., Bonner, T. I., et al. (2009). IUPHAR-DB: the IUPHAR database of G protein coupled receptors and ion channels. Nucleic Acids Res 37, D680-685.
Henderson, C., Wijetunge, L., Kinoshita, M. N., Shumway, M., Hammond, R. S., Postma, F. R., Brynczka, C., Rush, R., Thomas, A., Paylor, R., et al. (2012). Reversal of disease-related pathologies in the fragile X mouse model by selective activation of GABA(B) receptors with arbaclofen. Sci Transl Med 4, 152ra128.
Jones-Davis, D. M., Yang, M., Rider, E., Osbun, N. C., da Gente, G. J., Li, J., Katz, A. M., Weber, M. D., Sen, S., Crawley, J., and Sherr, E. H. (2013). Quantitative trait loci for interhemispheric commissure development and social behaviors in the BTBR T(+) tf/J mouse model of autism. PLoS One 8, e61829.
Li, X., Zou, H., and Brown, W. T. (2012). Genes associated with autism spectrum disorder. Brain Res Bull 88, 543-552.
Loscher, W., Rundfeldt, C., Honack, D., and Ebert, U. (1996). Long-term studies on anticonvulsant tolerance and withdrawal characteristics of benzodiazepine receptor ligands in different seizure models in mice. I. Comparison of diazepam, clonazepam, clobazam and abecarnil. J Pharmacol Exp Ther 279, 561-572.
Low, K., Crestani, F., Keist, R., Benke, D., Brunig, I., Benson, J. A., Fritschy, J. M., Rulicke, T., Bluethmann, H., Mohler, H., and Rudolph, U. (2000). Molecular and neuronal substrate for the selective attenuation of anxiety. Science 290, 131-134.
MacPherson, P., McGaffigan, R., Wahlsten, D., and Nguyen, P. V. (2008). Impaired fear memory, altered object memory and modified hippocampal synaptic plasticity in split-brain mice. Brain Res 1210, 179-188.
Markram, K., and Markram, H. (2010). The intense world theory—a unifying theory of the neurobiology of autism. Front Hum Neurosci 4, 224.
Mathiasen, L. S., Mirza, N. R., and Rodgers, R. J. (2008). Strain- and model-dependent effects of chlordiazepoxide, L-838,417 and zolpidem on anxiety-like behaviours in laboratory mice. Pharmacol Biochem Behav 90, 19-36.
McFarlane, H. G., Kusek, G. K., Yang, M., Phoenix, J. L., Bolivar, V. J., and Crawley, J. N. (2008). Autismlike behavioral phenotypes in BTBR T+tf/J mice. Genes Brain Behav 7, 152-163.
Michalon, A., Sidorov, M., Ballard, T. M., Ozmen, L., Spooren, W., Wettstein, J. G., Jaeschke, G., Bear, M. F., and Lindemann, L. (2012). Chronic pharmacological mGlu5 inhibition corrects fragile X in adult mice. Neuron 74, 49-56.
Peca, J., Feliciano, C., Ting, J. T., Wang, W., Wells, M. F., Venkatraman, T. N., Lascola, C. D., Fu, Z., and Feng, G. (2011). Shank3 mutant mice display autistic-like behaviours and striatal dysfunction. Nature 472, 437-442.
Penagarikano, O., Abrahams, B. S., Herman, E. I., Winden, K. D., Gdalyahu, A., Dong, H., Sonnenblick, L. I., Gruver, R., Almajano, J., Bragin, A., et al. (2011). Absence of CNTNAP2 leads to epilepsy, neuronal migration abnormalities, and core autism-related deficits. Cell 147, 235-246.
Prenosil, G. A., Schneider Gasser, E. M., Rudolph, U., Keist, R., Fritschy, J. M., and Vogt, K. E. (2006). Specific subtypes of GABA-A receptors mediate phasic and tonic forms of inhibition in hippocampal pyramidal neurons. J Neurophysiol 96, 846-857.
Rovira, C., and Ben-Ari, Y. (1993). Developmental study of benzodiazepine effects on monosynaptic GABA-A-mediated IPSPs of rat hippocampal neurons. J Neurophysiol 70, 1076-1085.
Rubenstein, J. L., and Merzenich, M. M. (2003). Model of autism: increased ratio of excitation/inhibition in key neural systems. Genes Brain Behav 2, 255-267.
Rudolph, U., and Knoflach, F. (2011). Beyond classical benzodiazepines: novel therapeutic potential of GABA-A receptor subtypes. Nat Rev Drug Discov 10, 685-697.
Rudolph, U., and Mohler, H. (2004). Analysis of GABA-A receptor function and dissection of the pharmacology of benzodiazepines and general anesthetics through mouse genetics. Annu Rev Pharmacol Toxicol 44, 475-498.
Savic, M. M., Obradovic, D. I., Ugresic, N. D., Cook, J. M., Yin, W., and Bokonjic, D. R. (2004).
Bidirectional effects of benzodiazepine binding site ligands in the elevated plus-maze: differential antagonism by flumazenil and beta-CCt. Pharmacol Biochem Behav 79, 279-290.
Savic, M. M., Obradovic, D. I., Ugresic, N. D., Cook, J. M., Yin, W., Van Linn, M., and Bokonjic, D. R. (2006). Benzodiazepine site inverse agonists and locomotor activity in rats: bimodal and biphasic influence. Pharmacol Biochem Behav 84, 35-42.
Sieghart, W. (1995). Structure and pharmacology of gamma-aminobutyric acidA receptor subtypes. Pharmacol Rev 47, 181-234.
Smith, G. B., and Olsen, R. W. (1995). Functional domains of GABA-A receptors. Trends Pharmacol Sci 16, 162-168.
Vinkers, C. H., van Oorschot, R., Nielsen, E. O., Cook, J. M., Hansen, H. H., Groenink, L, Olivier, B., and Mirza, N. R. (2012). GABA(A) receptor asubunits differentially contribute to diazepam tolerance after chronic treatment. PLoS One 7, e43054.
Yang, M., Abrams, D. N., Zhang, J. Y., Weber, M. D., Katz, A. M., Clarke, A. M., Silverman, J. L., and Crawley, J. N. (2012). Low sociability in BTBR T+tf/J mice is independent of partner strain. Physiol Behav 107, 649-662.
Yizhar, O., Fenno, L. E., Prigge, M., Schneider, F., Davidson, T. J., O'Shea, D. J., Sohal, V. S., Goshen, I., Finkelstein, J., Paz, J. T., et al. (2011). Neocortical excitation/inhibition balance in information processing and social dysfunction. Nature 477, 171-178.
Zoghbi, H. Y., and Bear, M. F. (2012). Synaptic dysfunction in neurodevelopmental disorders associated with autism and intellectual disabilities. Cold Spring Harb Perspect Biol 4.

### Methods—Supplemental

### Mice

The mice used for all behavioral analyses were 6-10 month-old adult male mice. As required for stable recordings of spontaneous synaptic activity, brain slices from 3-4 week-old mice were used for electrophysiological studies. All mice were singly housed at least 1 week before the behavioral tests. All experiments with animals were performed according to the National Institutes of Health Guide for Care and Use of Laboratory Animals and were approved by the University of Washington Institutional Animal Care and Use Committee.

### Control Mouse Strain

The inventors chose the C57BL/6J line as the control strain for comparison with BTBR based on precedents in the literature. At least fifty studies have used this mouse line as the primary control strain (Amodeo et al., 2012; Babineau et al., 2013; Benno et al., 2009; Blanchard et al., 2012; Bolivar et al., 2007; Cao et al., 2012; Chadman, 2011; Corley et al., 2012; Defensor et al., 2011; Ellegood et al., 2013; Frye and Llaneza, 2010; Gould et al., 2011; Heo et al., 2011; Jones-Davis et al., 2013; Karvat and Kimchi, 2012; Lipina and Roder, 2013; McFarlane et al., 2008; McTighe et al., 2013; Mercier et al., 2012; Meyza et al., 2012; Meyza et al., 2013; Miller et al., 2013; Moy et al., 2008; Moy et al., 2007; Onaivi et al., 2011; Pearson et al., 2012; Pearson et al., 2011; Pobbe et al., 2011; Pobbe et al., 2010; Roullet et al., 2011; Rutz and Rothblat, 2012; Scattoni et al., 2008; Scattoni et al., 2013; Scattoni et al., 2011; Schwartzer et al., 2013; Shah et al., 2013; Silverman et al., 2013a; Silverman et al., 2013b; Silverman et al., 2012; Silverman et al., 2010a; Silverman et al., 2010b; Stephenson et al., 2011; Wohr et al., 2011; Yang et al., 2012; Yang et al., 2009; Yang et al., 2011; Yang et al., 2007a; Yang et al., 2007b; Zhang et al., 2012; Zhang et al., 2013), whereas no other strain has emerged as a preferred alternative. The second most common type of control mouse strain, used in addition to C57BL/6J in five of these fifty studies, is the 129 mouse strain; however, different substrains of 129 mice were used in those five studies.

The inventors repeated the experiment with DMCM induction of autistic-like behaviors using 129SvJ mice and found comparable results to C57BL/6J (FIG. 41).

### Open-Field Test

Open field test was performed as previously described (Han et al., 2012). 50×50 cm square open field arena with non-transparent white Plexiglas was used in this study. During the 10 min of trial, total distance moved, circling behavior (a complete 360-degree turn of nose angle with respect to the body center) and time in center (20×20 cm imaginary square) were measured by the video-tracking software (EthoVision XT 8.5, Noldus Technology). All data shown are means±s.e.m. and analyzed using Student's two-tailed, unpaired t-test.

### Elevated Plus Maze Test

The test was performed as previously described (Han et al., 2012). The maze used in this study has two closed arms (5.1×30 cm) surrounded by 20-cm high non-transparent walls and two open arms (5.1×30 cm). During the 10 min of trial, times spent in closed, center, and open arms, and total distance traveled during the trial were measured by the video-tracking software (EthoVision XT 8.5, Noldus Technology). All data shown are means±s.e.m. and analyzed using Student's two-tailed, unpaired t-test.

### Three-Chamber Test

The test was performed as described previously (Han et al., 2012) with minor modifications. The threechamber apparatus is a non-transparent Plexiglas box (30×60 cm) with two transparent partitions that make left, center, and right chambers (30×20 cm). In the first 10-min session, a test mouse was placed in the center of the empty three-chamber unit to habituate the test mouse. The mouse was allowed to freely explore each chamber. In the second 10-min session, an age- and gender-matched same strain mouse that had never been exposed to the test mouse, was placed in one of the two wire cages. The empty wire cage as an inanimate object cue was placed on the other side. Then, the test mouse was placed again in the center, and allowed to freely explore the chamber for 10 min. Time spent in each chamber, and time spent within a 5 cm radius proximal to each wire cage, as a close interaction were measured by the videotracking software (EthoVision XT 8.5, Noldus Technology). All data shown are means±s.e.m. and analyzed using two-way ANOVA with Bonferroni's post hoc analysis and one-way ANOVA with Tukey's post hoc analysis.

### Reciprocal Interaction Test

A test mouse and an age- and gender-matched stimulus mouse with a mixed C57BL/6J×129 genetic background were introduced in an open field arena (50×50 cm). Mice were socially naïve with each other. The social interactions of mice were recorded by USB webcam (LifeCam HD-6000, Microsoft) and PC-based video capture software (WinAVI Video Capture, ZJ Media Digital Technology) for 10 min.

Time spent in close interaction (less than 5 cm proximity from the body center of each mouse), and time spent in nose-to-nose sniffing (less than 1 cm proximity) were measured automatically by the videotracking software (EthoVision XT 8.5, Noldus Technology). All data shown are means±s.e.m. and analyzed using two-way ANOVA with Bonferroni's post hoc analysis.

### Barnes Circular Maze Test

The test was performed as described previously (Han et al., 2012). The number of errors made and the latency to find the target hole were measured during the 3 min-training trials by video tracking software.

During the 90 second-probe trial, the latency to find the target hole, % correct pokes, and % time in the target area were measured by the video-tracking software (EthoVision XT 8.5, Noldus Technology). All data shown are means±s.e.m. and analyzed using two-way ANOVA with Bonferroni's post hoc analysis and Student's two-tailed, unpaired t-test.

### Contextual Fear Conditioning Test

The test was performed as described previously (Han et al., 2012). % freeze time, and the total distance moved during each 2 min session of habituation, training, 30 min test, and 24 h test were automatically measured by the video-tracking software (EthoVision XT 8.5, Noldus Technology). Freezing behavior is defined as the period during which the velocity of the test mouse is less than 1.75 cm/s, and non-freezing behavior is defined as the period during which the velocity of the mouse is greater than 2 cm/s. All data shown are means±s.e.m. and analyzed using Student's two-tailed, unpaired t-test.

### Brain Slice Electrophysiology

Hippocampal slices preparation and whole-cell voltage-clamp recordings were performed as described previously (Han et al., 2012).

### Drug Administration

Clonazepam and zolpidem tartrate (UDL Laboratories, Rockford, Ill.) were ground from tablets and suspended in phosphate buffered saline (PBS, Sigma), and vigorously vortexed immediately before the injection to prevent the precipitation of the drug particles. L-838,417, and DMCM (Sigma), dissolved in 100% DMSO (sterile, Sigma) was diluted in PBS (the final concentration of DMSO was ranged from 0.005% to 0.1%). Drugs were administered by an intraperitoneal injection in a volume of 0.01 ml/kg 30 min before the behavioral tests.

### Statistical Analysis

All data are shown as mean±s.e.m. and analyzed using Student's t-test, one-way ANOVA with Tukey's post hoc comparison, and two-way ANOVA with Bonferroni's post hoc comparison. All the statistical analyses were done using Prism 6 (GraphPad).

### REFERENCES

Amodeo, D. A., Jones, J. H., Sweeney, J. A., and Ragozzino, M. E. (2012). Differences in BTBR T+tf/J and C57BL/6J mice on probabilistic reversal learning and stereotyped behaviors. Behav Brain Res 227, 64-72.
Babineau, B. A., Yang, M., Berman, R. F., and Crawley, J. N. (2013). Low home cage social behaviors in BTBR T+tf/J mice during juvenile development. Physiol Behav 114-115, 49-54.
Benno, R., Smirnova, Y., Vera, S., Liggett, A., and Schanz, N. (2009). Exaggerated responses to stress in the BTBR T+tf/J mouse: an unusual behavioral phenotype. Behav Brain Res 197, 462-465.
Blanchard, D. C., Defensor, E. B., Meyza, K. Z., Pobbe, R. L., Pearson, B. L., Bolivar, V. J., and Blanchard, R. J. (2012). BTBR T+tf/J mice: autism-relevant behaviors and reduced fractone-associated heparin sulfate. Neurosci Biobehav Rev 36, 285-296.
Bolivar, V. J., Walters, S. R., and Phoenix, J. L. (2007). Assessing autism-like behavior in mice: variations in social interactions among inbred strains Behav Brain Res 176, 21-26.
Cao, F., Yin, A., Wen, G., Sheikh, A. M., Tauqeer, Z., Malik, M., Nagori, A., Schirripa, M., Schirripa, F., Merz, G., et al. (2012). Alteration of astrocytes and Wnt/beta-catenin signaling in the frontal cortex of autistic subjects. J Neuroinflammation 9, 223.
Chadman, K. K. (2011). Fluoxetine but not risperidone increases sociability in the BTBR mouse model of autism. Pharmacol Biochem Behav 97, 586-594.
Corley, M. J., Meyza, K. Z., Blanchard, D. C., and Blanchard, R. J. (2012). Reduced sulfate plasma concentrations in the BTBR T+tf/J mouse model of autism. Physiol Behav 107, 663-665.
Defensor, E. B., Pearson, B. L., Pobbe, R. L., Bolivar, V. J., Blanchard, D. C., and Blanchard, R. J. (2011). A novel social proximity test suggests patterns of social avoidance and gaze aversion-like behavior in BTBR T+tf/J mice. Behav Brain Res 217, 302-308.
Ellegood, J., Babineau, B. A., Henkelman, R. M., Lerch, J. P., and Crawley, J. N. (2013). Neuroanatomical analysis of the BTBR mouse model of autism using magnetic resonance imaging and diffusion tensor imaging. Neuroimage 70, 288-300.
Frye, C. A., and Llaneza, D. C. (2010). Corticosteroid and neurosteroid dysregulation in an animal model of autism, BTBR mice. Physiol Behav 100, 264-267.
Gould, G. G., Hensler, J. G., Burke, T. F., Benno, R. H., Onaivi, E. S., and Daws, L. C. (2011). Density and function of central serotonin (5-HT) transporters, 5-HT1A and 5-HT2A receptors, and effects of their targeting on BTBR T+tf/J mouse social behavior. J Neurochem 116, 291-303.
Heo, Y., Zhang, Y., Gao, D., Miller, V. M., and Lawrence, D. A. (2011). Aberrant immune responses in a mouse with behavioral disorders. PLoS One 6, e20912.
Jones-Davis, D. M., Yang, M., Rider, E., Osbun, N. C., da Gente, G. J., Li, J., Katz, A. M., Weber, M. D., Sen, S., Crawley, J., and Sherr, E. H. (2013). Quantitative trait loci for interhemispheric commissure development and social behaviors in the BTBR T(+) tf/J mouse model of autism. PLoS One 8, e61829.
Karvat, G., and Kimchi, T. (2012). Systematic autistic-like behavioral phenotyping of 4 mouse strains using a novel wheel-running assay. Behav Brain Res 233, 405-414.
Lipina, T. V., and Roder, J. C. (2013). Co-learning facilitates memory in mice: a new avenue in social neuroscience. Neuropharmacology 64, 283-293.
McFarlane, H. G., Kusek, G. K., Yang, M., Phoenix, J. L., Bolivar, V. J., and Crawley, J. N. (2008). Autismlike behavioral phenotypes in BTBR T+tf/J mice. Genes Brain Behav 7, 152-163.
McTighe, S. M., Neal, S. J., Lin, Q., Hughes, Z. A., and Smith, D. G. (2013). The BTBR mouse model of autism spectrum disorders has learning and attentional impairments and alterations in acetylcholine and kynurenic acid in prefrontal cortex. PLoS One 8, e62189.
Mercier, F., Kwon, Y. C., and Douet, V. (2012). Hippocampus/amygdala alterations, loss of heparin sulfates, fractones and ventricle wall reduction in adult BTBR T+tf/J mice, animal model for autism. Neurosci Lett 506, 208-213.
Meyza, K. Z., Blanchard, D. C., Pearson, B. L., Pobbe, R. L., and Blanchard, R. J. (2012). Fractone associated N-sulfated heparan sulfate shows reduced quantity in BTBR T+tf/J mice: a strong model of autism. Behav Brain Res 228, 247-253.
Meyza, K. Z., Defensor, E. B., Jensen, A. L., Corley, M. J., Pearson, B. L., Pobbe, R. L., Bolivar, V. J., Blanchard, D. C., and Blanchard, R. J. (2013). The BTBR T+tf/J mouse model for autism spectrum disorders-in search of biomarkers. Behav Brain Res 251, 25-34.
Miller, V. M., Gupta, D., Neu, N., Cotroneo, A., Boulay, C. B., and Seegal, R. F. (2013). Novel interhemispheric white matter connectivity in the BTBR mouse model of autism. Brain Res 1513, 26-33.
Moy, S. S., Nadler, J. J., Poe, M. D., Nonneman, R. J., Young, N. B., Koller, B. H., Crawley, J. N., Duncan, G. E., and Bodfish, J. W. (2008). Development of a mouse test for repetitive, restricted behaviors: relevance to autism. Behav Brain Res 188, 178-194.
Moy, S. S., Nadler, J. J., Young, N. B., Perez, A., Holloway, L. P., Barbaro, R. P., Barbaro, J. R., Wilson, L. M., Threadgill, D. W., Lauder, J. M., et al. (2007). Mouse behavioral tasks relevant to autism: phenotypes of 10 inbred strains. Behav Brain Res 176, 4-20.
Onaivi, E. S., Benno, R., Halpern, T., Mehanovic, M., Schanz, N., Sanders, C., Yan, X.,
Ishiguro, H., Liu, Q. R., Berzal, A. L., et al. (2011). Consequences of cannabinoid and monoaminergic system disruption in a mouse model of autism spectrum disorders. Curr Neuropharmacol 9, 209-214.
Pearson, B. L., Bettis, J. K., Meyza, K. Z., Yamamoto, L. Y., Blanchard, D. C., and Blanchard, R. J. (2012).
Absence of social conditioned place preference in BTBR T+tf/J mice: relevance for social motivation testing in rodent models of autism. Behav Brain Res 233, 99-104.
Pearson, B. L., Pobbe, R. L., Defensor, E. B., Oasay, L., Bolivar, V. J., Blanchard, D. C., and Blanchard, R. J. (2011). Motor and cognitive stereotypies in the BTBR T+tf/J mouse model of autism. Genes Brain Behav 10, 228-235.
Pobbe, R. L., Defensor, E. B., Pearson, B. L., Bolivar, V. J., Blanchard, D. C., and Blanchard, R. J. (2011).
General and social anxiety in the BTBR T+tf/J mouse strain. Behav Brain Res 216, 446-451.
Pobbe, R. L., Pearson, B. L., Defensor, E. B., Bolivar, V. J., Blanchard, D. C., and Blanchard, R. J. (2010).
Expression of social behaviors of C57BL/6J versus BTBR inbred mouse strains in the visible burrow system. Behav Brain Res 214, 443-449.
Roullet, F. I., Wohr, M., and Crawley, J. N. (2011). Female urine-induced male mice ultrasonic vocalizations, but not scent-marking, is modulated by social experience. Behav Brain Res 216, 19-28.
Rutz, H. L., and Rothblat, L. A. (2012). Intact and impaired executive abilities in the BTBR mouse model of autism. Behav Brain Res 234, 33-37.
Scattoni, M. L., Gandhy, S. U., Ricceri, L., and Crawley, J. N. (2008). Unusual repertoire of vocalizations in the BTBR T+tf/J mouse model of autism. PLoS One 3, e3067.
Scattoni, M. L., Martire, A., Cartocci, G., Ferrante, A., and Ricceri, L. (2013). Reduced social interaction, behavioural flexibility and BDNF signalling in the BTBR T+tf/J strain, a mouse model of autism. Behav Brain Res 251, 35-40.
Scattoni, M. L., Ricceri, L., and Crawley, J. N. (2011). Unusual repertoire of vocalizations in adult BTBR T+tf/J mice during three types of social encounters. Genes Brain Behav 10, 44-56.
Schwartzer, J. J., Careaga, M., Onore, C. E., Rushakoff, J. A., Berman, R. F., and Ashwood, P. (2013). Maternal immune activation and strain specific interactions in the development of autism-like behaviors in mice. Transl Psychiatry 3, e240.
Shah, C. R., Forsberg, C. G., Kang, J. Q., and Veenstra-VanderWeele, J. (2013). Letting a typical mouse judge whether mouse social interactions are atypical. Autism Res 6, 212-220.
Silverman, J. L., Babineau, B. A., Oliver, C. F., Karras, M. N., and Crawley, J. N. (2013a). Influence of stimulant-induced hyperactivity on social approach in the BTBR mouse model of autism. Neuropharmacology 68, 210-222.
Silverman, J. L., Oliver, C. F., Karras, M. N., Gastrell, P. T., and Crawley, J. N. (2013b). AMPAKINE enhancement of social interaction in the BTBR mouse model of autism. Neuropharmacology 64, 268-282.
Silverman, J. L., Smith, D. G., Rizzo, S. J., Karras, M. N., Turner, S. M., Tolu, S. S., Bryce, D. K., Smith, D. L., Fonseca, K., Ring, R. H., and Crawley, J. N. (2012). Negative allosteric modulation of the mGluR5 receptor reduces repetitive behaviors and rescues social deficits in mouse models of autism. Sci Transl Med 4, 131ra151.
Silverman, J. L., Tolu, S. S., Barkan, C. L., and Crawley, J. N. (2010a). Repetitive self-grooming behavior in the BTBR mouse model of autism is blocked by the mGluR5 antagonist MPEP. Neuropsychopharmacology 35, 976-989.
Silverman, J. L., Yang, M., Turner, S. M., Katz, A. M., Bell, D. B., Koenig, J. I., and Crawley, J. N. (2010b). Low stress reactivity and neuroendocrine factors in the BTBR T+tf/J mouse model of autism. Neuroscience 171, 1197-1208.
Stephenson, D. T., O'Neill, S. M., Narayan, S., Tiwari, A., Arnold, E., Samaroo, H. D., Du, F., Ring, R. H., Campbell, B., Pletcher, M., et al. (2011). Histopathologic characterization of the BTBR mouse model of autistic-like behavior reveals selective changes in neurodevelopmental proteins and adult hippocampal neurogenesis. Mol Autism 2, 7.
Wohr, M., Roullet, F. I., and Crawley, J. N. (2011). Reduced scent marking and ultrasonic vocalizations in the BTBR T+tf/J mouse model of autism. Genes Brain Behav 10, 35-43.
Yang, M., Abrams, D. N., Zhang, J. Y., Weber, M. D., Katz, A. M., Clarke, A. M., Silverman, J. L., and Crawley, J. N. (2012). Low sociability in BTBR T+tf/J mice is independent of partner strain. Physiol Behav 107, 649-662.
Yang, M., Clarke, A. M., and Crawley, J. N. (2009). Postnatal lesion evidence against a primary role for the corpus callosum in mouse sociability. Eur J Neurosci 29, 1663-1677.
Yang, M., Perry, K., Weber, M. D., Katz, A. M., and Crawley, J. N. (2011). Social peers rescue autism relevant sociability deficits in adolescent mice. Autism Res 4, 17-27.
Yang, M., Scattoni, M. L., Zhodzishsky, V., Chen, T., Caldwell, H., Young, W. S., McFarlane, H. G., and Crawley, J. N. (2007a). Social approach behaviors are similar on conventional versus reverse lighting cycles, and in replications across cohorts, in BTBR T+tf/J, C57BL/6J, and vasopressin receptor 1B mutant mice. Front Behav Neurosci 1, 1.
Yang, M., Zhodzishsky, V., and Crawley, J. N. (2007b). Social deficits in BTBR T+tf/J mice are unchanged by cross-fostering with C57BL/6J mothers. Int J Dev Neurosci 25, 515-521.
Zhang, Y., Bolivar, V. J., and Lawrence, D. A. (2012). Developmental exposure to mercury chloride does not impair social behavior of C57BL/6×BTBR F(1) mice. J Immunotoxicol 9, 401-410.
Zhang, Y., Gao, D., Kluetzman, K., Mendoza, A., Bolivar, V. J., Reilly, A., Jolly, J. K., and Lawrence, D. A. (2013). The maternal autoimmune environment affects the social behavior of offspring. J Neuroimmunol 258, 51-60.

1. A method for treating an Autism Spectrum Disorder (ASD) or an indicium thereof, the method comprising
administering a low dose of an agent that increases GABAergic signaling to a subject having an ASD, thereby treating the ASD in the subject.

2-49. (canceled)

50. The method of claim 1, wherein the agent that increases GABAergic signaling is a positive allosteric modulator of the GABA-A receptor.

51. The method of claim 50, wherein the positive allosteric modulator of the GABA-A receptor has efficacy at GABA-A receptors comprising an α2 and/or α3 subunit.

52. The method of claim 50, wherein the positive allosteric modulator of the GABA-A receptor is selective for a GABA-A receptor comprising an α2 and/or α3 subunits.

53. The method of claim 50, wherein the agent that increases GABAergic signaling is a benzodiazepine, or a non-benzodiazepine enhancer at the GABA-A receptor (non-BDZ GABA-A enhancer).

54. The method of claim 50, wherein the benzodiazepine is a full agonist or a partial agonist at the GABA-A receptor.

55. The method of claim 53, wherein the benzodiazepine is a short-acting or long-acting benzodiazepine.

56. The method of claim 53, wherein the benzodiazepine is clonazepam or clobazam.

57. The method of claim 53, wherein the non-BDZ GABA-A enhancer is L838,417.

58. The method of claim 53, wherein the non-BDZ GABA-A enhancer is a neurosteroid.

59. The method of claim 1, wherein the dose of the agent that increases GABAergic signaling is less than the dose of the same agent that causes sedation, anticonvulsive effects, or anxiolytic effects in a subject.

60. The method of claim 1, wherein the dose of the agent that increases GABAergic signaling is about 10% of the dose of the same agent that causes sedation, anticonvulsive effects or anxiolytic effects in a subject.

61. The method of claim 1, wherein the ASD comprises at least one symptom selected from the group consisting of: poor social interactions, repetitive behaviors, cognitive deficit and impaired language development.

62. A method for reducing or ameliorating at least one indicium of an Autism Spectrum Disorder (ASD), the method comprising: administering a sub-sedative, sub-anxiolytic or sub-anticonvulsive dose of an agent that increases the response of the GABA-A receptor to GABA, whereby at least one indicium is reduced or ameliorated.

62. The method of claim 62, wherein the at least one indicium of an Autism Spectrum Disorder is selected from the group consisting of: repetitive behavior(s), impaired social interactions, cognitive deficit and impaired language development.

64. The method of claim 62, wherein the at least one indicium of an Autism Spectrum Disorder is repetitive behavior(s) and/or impaired social interactions.

65. The method of claim 62, wherein the agent is a benzodiazepine, a neurosteroid, a subunit-selective positive allosteric modulator of GABA-A, or a non-BDZ GABA-A enhancer.

66. A composition comprising a low-dose formulation of a benzodiazepine and a pharmaceutically acceptable carrier, wherein the dose of the benzodiazepine is less than the dose of the same benzodiazepine that causes sedation, anticonvulsive effects or anxiolytic effects in a subject.

67. The composition of claim 66, wherein the dose of the benzodiazepine is less than 20% of the dose of the same benzodiazepine that causes sedation, anticonvulsive effects or anxiolytic effects in a subject.

68. The composition of claim 66, wherein the dose of the benzodiazepine is less than 10% of the dose of the same benzodiazepine that causes sedation, anticonvulsive effects or anxiolytic effects in a subject.

69. The composition of claim 66, wherein the composition is in the form of a tablet, a capsule, a suspension, or a solution.

70. The composition of claim 66, wherein the benzodiazepine is a full agonist or a partial agonist at the GABA-A receptor.

71. The composition of claim 66, wherein the benzodiazepine has efficacy at a GABA-A receptor comprising an α2 and/or α3 subunit.

72. The composition of claim 66, wherein the benzodiazepine is selective for a GABA-A receptor comprising an α2 and/or α3 subunit.

73. The composition of claim 66, wherein the benzodiazepine is a short-acting or long-acting benzodiazepine.

74. The composition of claim 66, wherein the benzodiazepine is clonazepam and the composition comprises a dose of clonazepam within the range of 0.01 mg to 0.05 mg.

75. A composition comprising a low-dose formulation of a non-benzodiazepine GABA-A enhancer (non-BDZ GABA-A enhancer) and a pharmaceutically acceptable carrier, wherein the dose of the non-BDZ GABA-A enhancer is less than the dose of the same non-BDZ GABA-A enhancer that causes sedation, anticonvulsive effects or anxiolytic effects in a subject.

76. The composition of claim 75, wherein the dose of the non-BDZ GABA-A enhancer is less than 20% of the dose of the same non-BDZ GABA-A enhancer that causes sedation, anticonvulsive effects or anxiolytic effects in a subject.

77. The composition of claim 75, wherein the dose of the non-BDZ GABA-A enhancer is less than 10% of the dose of the same non-BDZ GABA-A enhancer that causes sedation, anticonvulsive effects or anxiolytic effects in a subject.

78. The composition of claim 75, wherein the non-BDZ GABA-A enhancer has efficacy at a GABA-A receptor comprising an α2 and/or α3 subunit.

79. The composition of claim 75, wherein the non-BDZ GABA-A enhancer is selective for a GABA-A receptor comprising an α2 and/or α3 subunit.

80. The composition of claim 75, wherein the non-BDZ GABA-A enhancer is L838,417.

81. The composition of claim 75, wherein the non-BDZ GABA-A enhancer is a neurosteroid.

82. The composition of claim 75, wherein the composition is in the form of a tablet, a capsule, a suspension, or a solution.
